(19) United States
(12) Patent Application Publication
(10) Pub. No.: US 2017/0349540 A1
(43) Pub. Date: Dec. 7, 2017

(54) HISTONE DEACETYLASE INHIBITORS

(71) Applicant: The General Hospital Corporation, Boston, MA (US)

(72) Inventors: Jacob M. Hooker, Belmont, MA (US); Changning Wang, Melrose, MA (US); Frederick A. Schroeder, Baltimore, MD (US)

(21) Appl. No.: 15/329,346

(22) PCT Filed: Jul. 27, 2015
(86) PCT No.: PCT/US15/42206

§ 371 Date: Jan. 26, 2017

Related U.S. Application Data
(60) Provisional application No. 62/029,900, filed on Jul. 28, 2014.
(60) Provisional application No. 62/042,046, filed on Aug. 26, 2014.

(51) Int. Cl. C07C 259/06; C07C 237/20; C07C 2603/74

(52) U.S. Cl. CPC C07C 259/06; C07C 237/20; C07C 2603/74

(57) ABSTRACT

Provided herein are brain penetrant histone deacetylase (HDAC) inhibitors useful for treating diseases or disorders associated with HDAC. An exemplary HDAC inhibitor provided herein exhibits a brain-to-plasma ratio of 20:1. Pharmaceutical compositions comprising HDAC inhibitors and methods for treating diseases associated with HDAC are also provided.

### CROSS-REFERENCE TO RELATED APPLICATIONS

This application claims the benefit of U.S. Provisional Application Ser. No. 62/029,900, filed Jul. 28, 2014, and 62/042,046, filed Aug. 26, 2014, the disclosures of each of which are incorporated herein by reference in their entirety.

### FEDERALLY SPONSORED RESEARCH OR DEVELOPMENT

This invention was made with Government support under Grant No. R01 DA030321 awarded by the National Institutes of Health. The Government has certain rights in the invention.

### TECHNICAL FIELD

This invention relates to histone deacetylase inhibitors (HDAC), and more particularly to brain-penetrant inhibitors that are useful for treatment of HDAC related disorders.

### BACKGROUND

Histone deacetylases (HDACs) are a family of chromatin modifying enzymes that modulate DNA packaging, gene expression and have been linked to biological functions from differentiation at the cellular level to higher-order brain function via behavioral changes at the organismal level. Evidence increasingly supports that targeting epigenetic mechanisms and chromatin-mediated neuroplasticity may improve treatments for neuropsychiatric diseases.

### SUMMARY

The present application provides, inter alia, a compound of Formula (I):

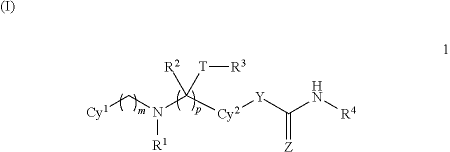

or a pharmaceutically acceptable salt thereof, wherein:

each T is independently absent or a C₁₋₄ alkylene;

Y is absent, C₁₋₄ alkylene, or C₂₋₄ alkenylene;

Z is selected from the group consisting of O, S, and NR^{b};

R¹ is selected from the group consisting of H, C₁₋₆ alkyl, C₁₋₆ haloalkyl, C₂₋₆ alkenyl, C₂₋₆ alkynyl, and OR^{a}; wherein the C₁₋₆ alkyl, C₂₋₆ alkenyl, and C₂₋₆ alkynyl are each optionally substituted with 1, 2, 3, or 4 independently selected R⁶ groups;

each R² is independently selected from the group consisting of absent, H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}; wherein the C₁₋₆ alkyl, C₂₋₆ alkenyl, and C₂₋₆ alkynyl are each optionally substituted with 1, 2, 3, or 4 independently selected R⁶ groups;

each R³ is independently selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, —S(═O)₂R^{c}, (═O), (═S), and (═NR^{b}); wherein the C₁₋₆ alkyl, C₂₋₆ alkenyl, and C₂₋₆ alkynyl are each optionally substituted with 1, 2, 3, or 4 independently selected R⁶ groups;

R⁴ is selected from the group consisting of H, C₁₋₆ alkyl, C₁₋₆ haloalkyl, C₂₋₆ alkenyl, C₂₋₆ alkynyl, OR^{a}, and Cy³; wherein the C₁₋₆ alkyl, C₂₋₆ alkenyl, and C₂₋₆ alkynyl are each optionally substituted with 1, 2, 3, or 4 independently selected R⁶ groups;

each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

each R^{a}, R^{b}, and R^{c} is independently H or C₁₋₆ alkyl;

Cy¹ is selected from the group consisting of C₃₋₁₀ cycloalkyl, 4-10 membered heterocycloalkyl, C₆₋₁₀ aryl, and 4-10 membered heteroaryl; wherein the C₃₋₁₀ cycloalkyl, 4-10 membered heterocycloalkyl, C₆₋₁₀ aryl, and 4-10 membered heteroaryl are each optionally substituted by 1, 2, 3, or 4 substituents independently selected from C₁₋₆ alkyl, C₁₋₆ haloalkyl, C₁₋₄ alkoxy, halo, OH, and CN;

Cy² is selected from the group consisting of C₆₋₁₀ aryl, C₃₋₁₀ cycloalkyl, 4-10 membered heteroaryl, and 4-10 membered heterocycloalkyl; wherein said C₆₋₁₀ aryl, C₃₋₁₀ cycloalkyl, 4-10 membered heteroaryl, and 4-10 membered heterocycloalkyl are each optionally substituted by 1, 2, 3, or 4 substituents independently selected from C₁₋₆ alkyl, C₁₋₆ haloalkyl, C₁₋₄ alkoxy, halo, OH, and CN;

Cy³ is selected from the group consisting of C₃₋₁₀ cycloalkyl, 4-10 membered heterocycloalkyl, C₆₋₁₀ aryl, and 4-10 membered heteroaryl; wherein the C₃₋₁₀ cycloalkyl, 4-10 membered heterocycloalkyl, C₆₋₁₀ aryl, and 4-10 membered heteroaryl are each substituted by 1, 2, 3, or 4 substituents independently selected from halo, OH, CN, C₁₋₆ alkyl, C₁₋₆ haloalkyl, C₁₋₄ alkoxy, C₆₋₁₀ aryl, 4-10 membered heteroaryl, amino, C₁₋₃ alkylamino, di(C₁₋₃ alkyl)amino, carbamyl, C₁₋₃ alkylcarbamyl, di(C₁₋₃ alkyl)carbamyl, C₁₋₃ alkylcarbonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

m is 0, 1, 2, 3, 4, 5, or 6; and

p is 0, 1, 2, 3, 4, 5, or 6,

with the proviso that the compound of Formula (I), is not selected from the group consisting of:

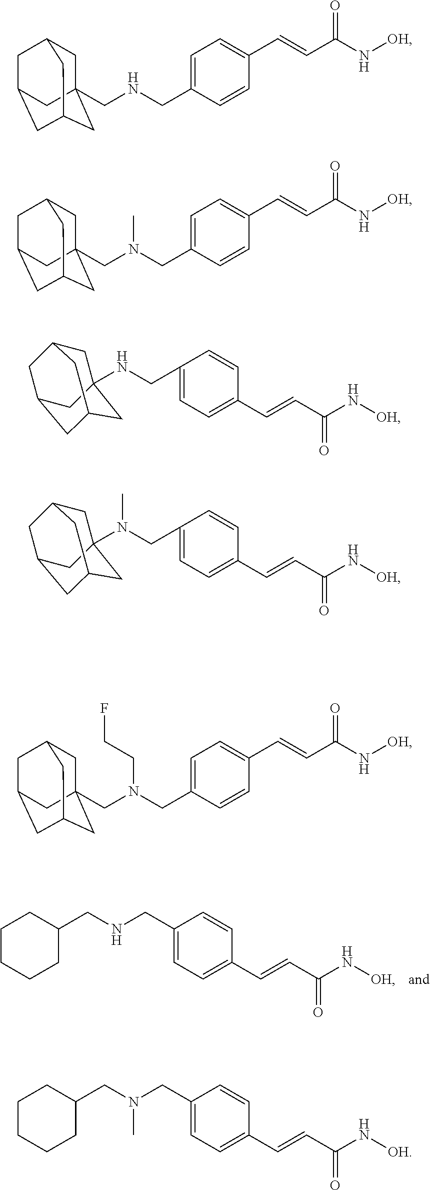

In some embodiments, m is 0, 1, or 2 and p is 1. In some embodiments, m is 0 and p is 1. In some embodiments, m is 1, and p is 1. In some embodiments, m is 2 and p is 1.

In some embodiments, Y is absent or C₂₋₄ alkenylene. In some embodiments, Y is C₂₋₄ alkenylene. In some embodiments, Y is —CH═CH—. In some embodiments, Y is:

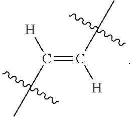 [SMILES: */C=C/*]

In some embodiments, Y is absent.

In some embodiments, wherein Z is O.

In some embodiments, R¹ is selected from the group consisting of H, C₁₋₆ alkyl, and C₁₋₆ haloalkyl. In some embodiments, R¹ is selected from the group consisting of H, —CH₃, and —CH₂CH₂F. In some embodiments, R¹ is H. In some embodiments, R¹ is —CH₃.

In some embodiments, R¹ is —CH₂CH₂F.

In some embodiments, each R² is independently selected from the group consisting of absent, H, C₁₋₆ alkyl, and C₁₋₆ haloalkyl. In some embodiments, each R² is H.

In some embodiments, each R³ is independently selected from the group consisting of H, C₁₋₆ alkyl, C₁₋₆ haloalkyl, OR^{a}, (═O), (═S), and (═NR^{b}). In some embodiments, each R³ is independently selected from the group consisting of H, OR^{a}, and (═O). In some embodiments, each R³ is independently H or (═O). In some embodiments, each R³ is H. In some embodiments, each R³ is (═O).

In some embodiments, R⁴ is selected from the group consisting of H, OH, or Cy³.

In some embodiments, R⁴ is OH or Cy³. In some embodiments, R⁴ is Cy³. In some embodiments, R⁴ is a C₆₋₁₀ aryl or a 4-10 membered heteroaryl ring. In some embodiments, R⁴ is C₆₋₁₀ aryl. In some embodiments, R⁴ is phenyl. In some embodiments, R⁴ is phenyl, wherein the phenyl is substituted by 1, 2, 3, or 4 substituents independently selected from halo, OH, CN, C₁₋₆ alkyl, C₁₋₆ haloalkyl, C₁₋₄ alkoxy, C₆₋₁₀ aryl, 4-10 membered heteroaryl amino, C₁₋₃ alkylamino, and di(C₁₋₃ alkyl)amino. In some embodiments, R⁴ is phenyl, wherein the phenyl is substituted by 1, 2, 3, or 4 substituents independently selected from amino, C₁₋₃ alkylamino, and di(C₁₋₃ alkyl)amino. In some embodiments, R⁴ is phenyl, wherein the phenyl is substituted by 1 amino group. In some embodiments, R⁴ is:

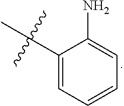 [SMILES: *c1ccccc1N]

In some embodiments, R⁴ is phenyl, wherein the phenyl is substituted by 1 or 2 substituents independently selected from the group consisting of halo, OH, CN, C₁₋₆ alkyl, C₁₋₆ haloalkyl, C₁₋₄ alkoxy, C₆₋₁₀ aryl, 4-10 membered heteroaryl, and amino. In some embodiments, R⁴ is phenyl, wherein the phenyl is substituted by 2 substituents independently selected from the group consisting of halo, OH, CN, C₁₋₆ alkyl, C₁₋₆ haloalkyl, C₁₋₄ alkoxy, C₆₋₁₀ aryl, 4-10 membered heteroaryl, and amino. In some embodiments, R⁴ is phenyl, wherein the phenyl is substituted by 2 substituents independently selected from the group consisting of halo, C₆₋₁₀ aryl, 4-10 membered heteroaryl, and amino. In some embodiments, R⁴ is phenyl, wherein the phenyl is substituted by 2 substituents independently selected from the group consisting of fluoro, phenyl, thiophene, and amino. In some embodiments, R⁴ is selected from the group consisting of:

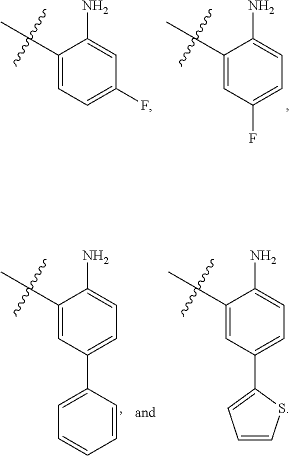

In some embodiments, R⁴ is OH.

In some embodiments, each R⁶ is independently selected from the group consisting of OH, NO₂, CN, halo, and C₁₋₃ alkyl.

In some embodiments, Cy¹ is a C₃₋₁₀ cycloalkyl or a 4-10 membered heterocycloalkyl. In some embodiments, Cy¹ is C₃₋₁₀ cycloalkyl. In some embodiments, Cy¹ is cyclohexyl or adamantyl. In some embodiments, Cy¹ is cyclohexyl. In some embodiments, Cy¹ is adamantyl.

In some embodiments, Cy² is C₆₋₁₀ aryl or 4-10 membered heterocyloalkyl. In some embodiments, Cy² is phenyl. In some embodiments, Cy² is azetidinyl.

In some embodiments, each T is independently absent or —CH₂—; Y is C₂₋₆ alkenylene; Z is O; R¹ is selected from the group consisting of H, C₁₋₆ alkyl, and C₁₋₆ haloalkyl; each R² is independently H or absent; each R³ is independently H or (═O); R⁴ is OH or Cy³; Cy¹ is C₃₋₁₀ cycloalkyl; Cy² is C₆₋₁₀ aryl or 4-10 membered heterocycloalkyl; Cy³ is C₆₋₁₀ aryl; m is 0, 1, or 2; and p is 1.

In some embodiments, T is independently absent or —CH₂—; Y is —CH═CH—; Z is O; R¹ is selected from the group consisting of H, —CH₃, and —CH₂CH₂F; each R² is independently H or absent; each R³ is independently H or (═O); R⁴ is OH or Cy³; Cy¹ is cyclohexyl or adamantyl; Cy² is phenyl or azetidinyl; Cy³ is phenyl, wherein the phenyl is substituted by 1 amino group; m is 0, 1, or 2; and p is 1.

In some embodiments, T and R² are each absent; Y is —CH═CH—; Z is O; R¹ is selected H; R³ is (═O); R⁴ is OH or Cy³; Cy¹ is C₃₋₁₀ cycloalkyl; Cy² is phenyl or azetidinyl; Cy³ is phenyl, wherein the phenyl is substituted by 1 amino group; m is 1 or 2; and p is 1.

In some embodiments, T is absent; Y is —CH═CH—; R¹ is selected from the group consisting of H, —CH₃, and —CH₂CH₂F; R² is H; R³ is H; R⁴ is OH or Cy³; Cy¹ is C₃₋₁₀ cycloalkyl; Cy² is C₆₋₁₀ aryl or 4-10 membered heterocycloalkyl; Cy³ is phenyl, wherein the phenyl is substituted by 1 amino group; m is 0 or 1; and p is 1.

In some embodiments, T is absent; Y is —CH═CH—; R¹ is selected from the group consisting of H, —CH₃, and —CH₂CH₂F; R² is H; R³ is H; R⁴ is OH or Cy³; Cy¹ is cyclohexyl or adamantyl; Cy² is phenyl or azetidinyl; Cy³ is phenyl, wherein the phenyl is substituted by 1 amino group; m is 0 or 1; and p is 1.

In some embodiments, Cy¹ is cyclohexyl or adamantyl; Cy² is phenyl or azetidinyl; R⁴ is Cy³; and Cy³ is phenyl, wherein the phenyl is substituted by 1 amino group.

In some embodiments, Cy¹ is cyclohexyl or adamantyl; Cy² is phenyl or azetidinyl; and R⁴ is OH.

In some embodiments, Cy¹ is adamantyl; Cy² is phenyl or azetidinyl; R⁴ is Cy³; and Cy³ is phenyl, wherein the phenyl is substituted by 2 substituents independently selected from the group consisting of halo, C₆₋₁₀ aryl, 4-10 membered heteroaryl, and amino.

In some embodiments, the compound of Formula (I) is a compound of Formula (II):

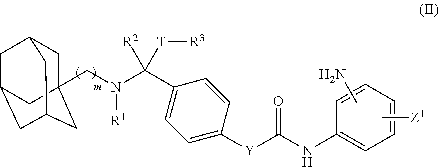

or a pharmaceutically acceptable salt thereof, wherein:

Z¹ is absent or selected from the group consisting of halo, OH, CN, C₁₋₆ alkyl, C₁₋₆ haloalkyl, C₁₋₄ alkoxy, C₆₋₁₀ aryl, 4-10 membered heteroaryl.

In some embodiments, the compound of Formula (I) is a compound of Formula (III):

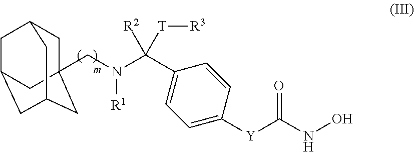

or a pharmaceutically acceptable salt thereof.

In some embodiments, the compound of Formula (I) is a compound of Formula (IV):

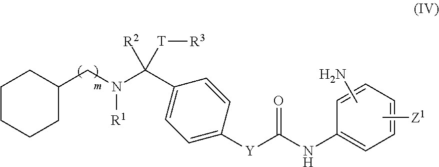

or a pharmaceutically acceptable salt thereof, wherein:

Z¹ is absent or selected from the group consisting of halo, OH, CN, C₁₋₆ alkyl, C₁₋₆ haloalkyl, C₁₋₄ alkoxy, C₆₋₁₀ aryl, 4-10 membered heteroaryl.

In some embodiments, the compound of Formula (I) is a compound of Formula (V):

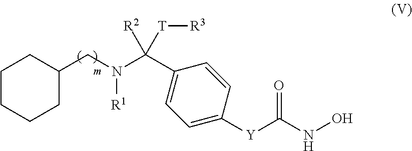

or a pharmaceutically acceptable salt thereof.

In some embodiments, compound of Formula (I) is selected from the group consisting of:

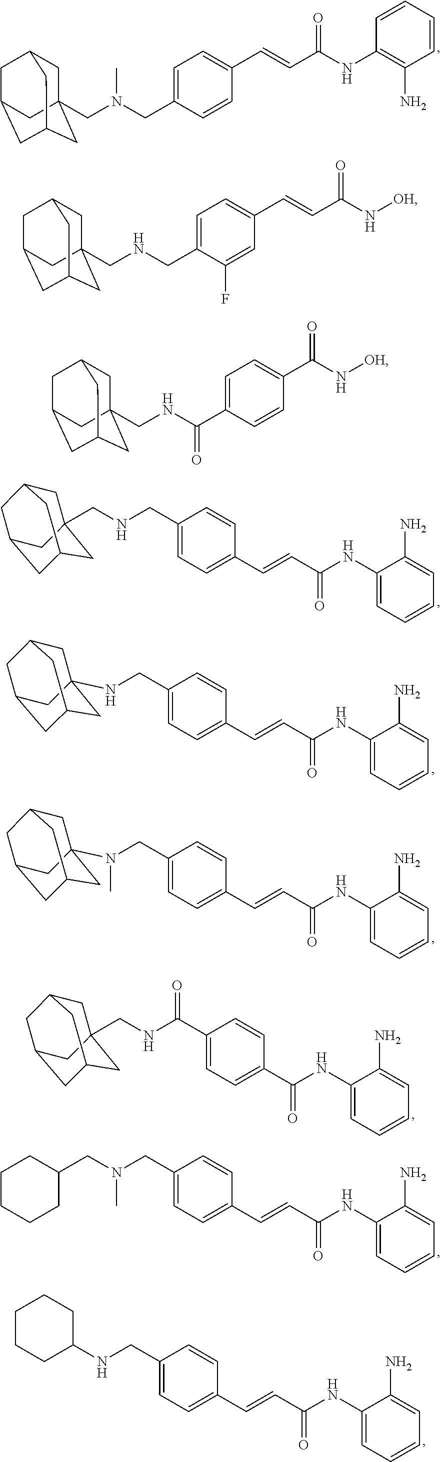

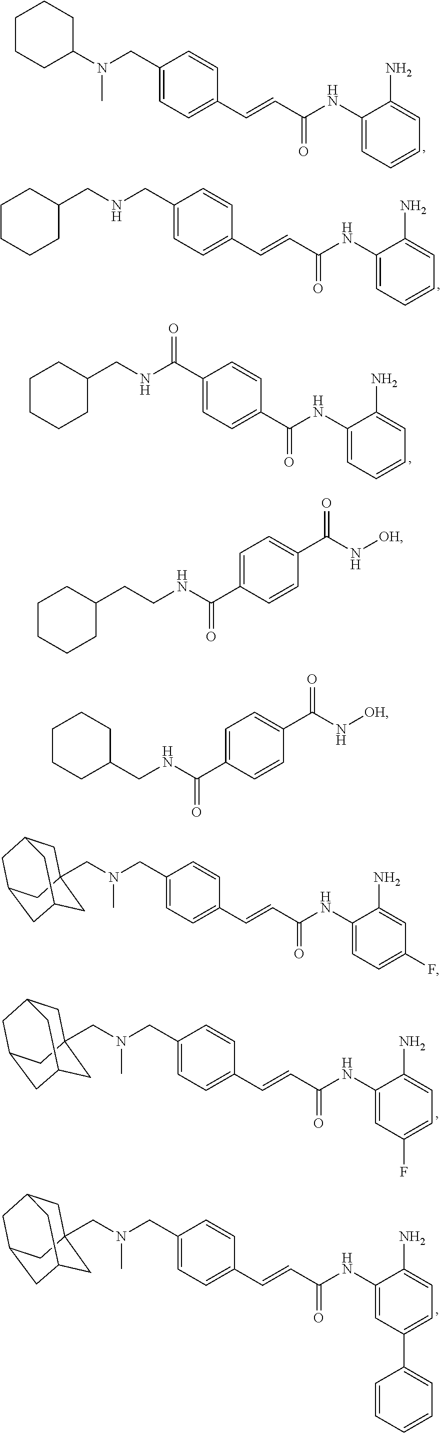

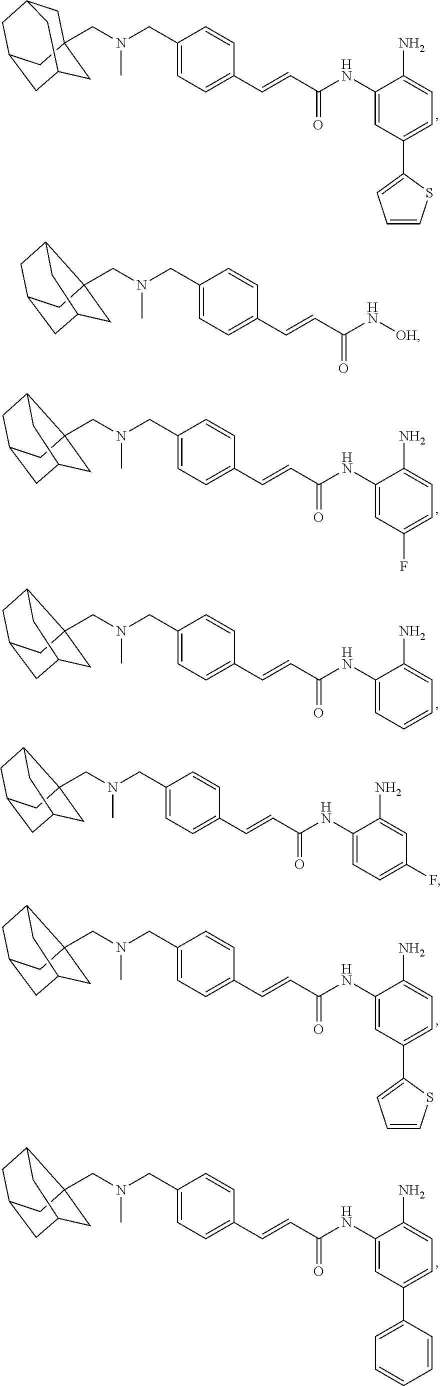

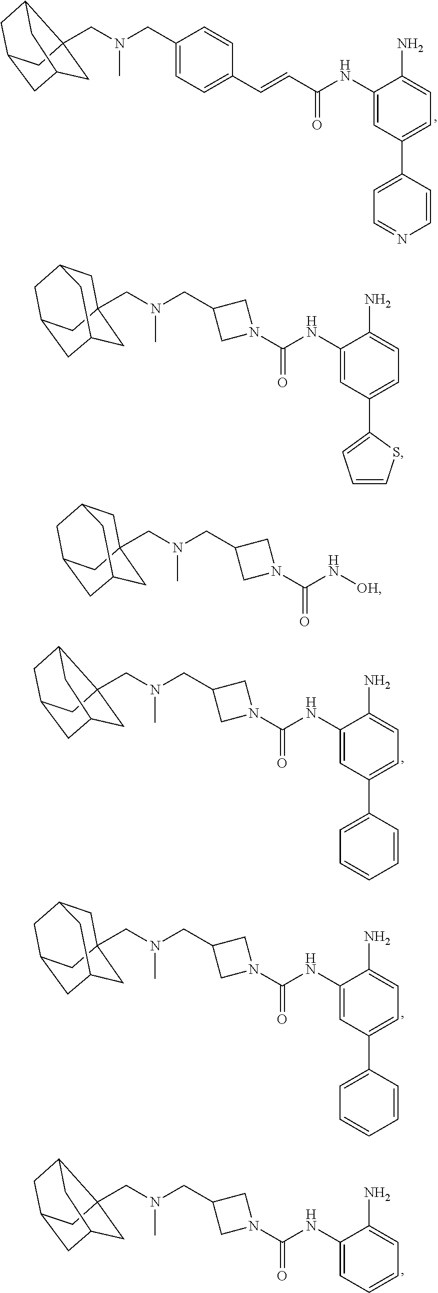 [SMILES: CN(Cc1ccc(/C=C/C(=O)Nc2cc(-c3ccncc3)ccc2N)cc1)CC12CC3CC(CC1C3)C2 ; CN(CC1CN(C(=O)Nc2cc(-c3cccs3)ccc2N)C1)CC12CC3CC(CC(C3)C1)C2 ; CN(CC1CN(C(=O)NO)C1)CC12CC3CC(CC(C3)C1)C2 ; CN(CC1CN(C(=O)Nc2cc(-c3ccccc3)ccc2N)C1)CC12CC3CC(CC1C3)C2 ; CN(CC1CN(C(=O)Nc2cc(-c3ccccc3)ccc2N)C1)CC12CC3CC(CC(C3)C1)C2 ; CN(CC1CN(C(=O)Nc2ccccc2N)C1)CC12CC3CC(CC1C3)C2]

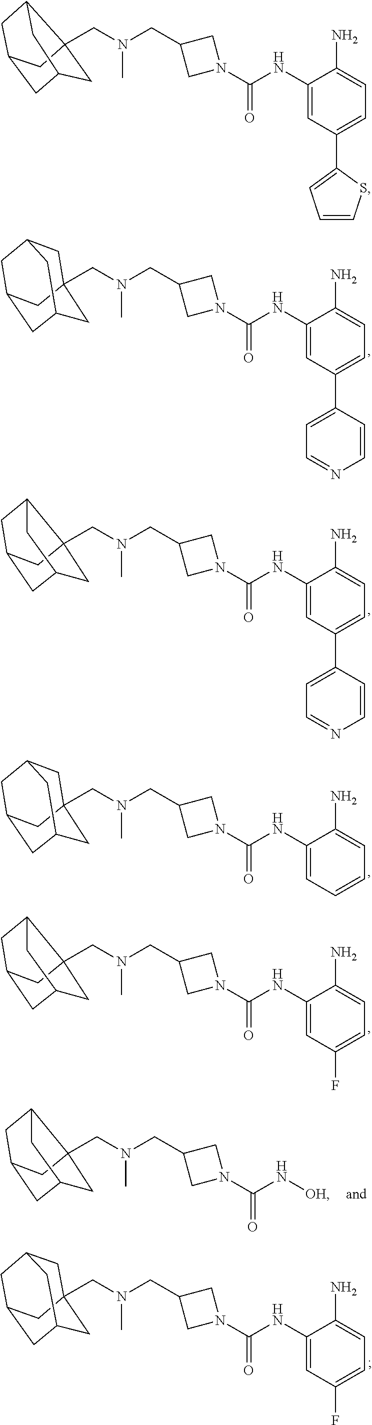

or a pharmaceutically acceptable salt thereof.

In some embodiments, the compound of Formula (I) is:

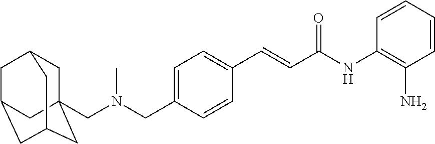 [SMILES: CN(Cc1ccc(/C=C/C(=O)Nc2ccccc2N)cc1)CC12CC3CC(CC(C3)C1)C2]

or a pharmaceutically acceptable salt thereof.

Also provided is a pharmaceutical composition comprising a compound of Formula (I), or a pharmaceutically acceptable salt thereof, an at least one pharmaceutically acceptable carrier.

In some embodiments, the pharmaceutical composition comprises the compound:

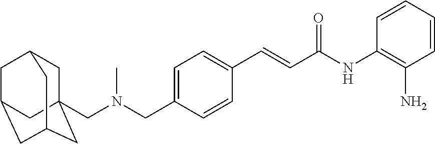 [SMILES: CN(Cc1ccc(/C=C/C(=O)Nc2ccccc2N)cc1)CC12CC3CC(CC(C3)C1)C2]

or a pharmaceutically acceptable salt thereof.

Also provided is a method of inhibiting an activity of a histone deacetylase (HDAC) enzyme, comprising contacting the HDAC enzyme with a compound of Formula (I), or a pharmaceutically acceptable salt thereof.

In some embodiments, inhibiting an activity of a histone deacetylase (HDAC) enzyme comprises deregulating the histone deacetylase (HDAC) enzyme.

In some embodiments, the histone deacetylase (HDAC) enzyme is a Class I HDAC enzyme. In some embodiments, the compound selectively inhibits HDAC1 and HDAC2 over HDAC3. In some embodiments, the compound selectively inhibits HDAC3 over HDAC1 and HDAC2.

In some embodiments, two or more of the compounds of Formula (I) are contacted.

In some embodiments, the compound is administered in combination with a chemotherapeutic agent, an immunosuppressant, or an anti-inflammatory agent. In some embodiments, the compound is administered in combination with a chemotherapeutic agent. In some embodiments, the compound is administered in combination with an immunosuppressant. In some embodiments, the compound is administered in combination with an anti-inflammatory agent.

Also provided is a method of treating a disease in a patient in need thereof, the method comprising administering to the patient a therapeutically effective amount of a compound of Formula (I), or a pharmaceutically acceptable salt thereof, wherein the disease is selected from the group consisting of cancer, a disease of the central nervous system, and an inflammatory autoimmune disease.

In some embodiments, the disease is cancer. In some embodiments, the cancer comprises a solid tumor. In some embodiments, the cancer is selected from the group consisting of glioma, glioblastoma, hematological cancer, and non-small cell lung cancer.

In some embodiments, the cancer is a hematological cancer. In some embodiments, the cancer is a hematological cancer selected from leukemia or lymphoma. In some embodiments, the cancer is glioma. In some embodiments, the cancer is glioblastoma. In some embodiments, the cancer is non-small cell lung cancer. In some embodiments, the cancer is associated with abnormal expression or abnormal activity of a histone deacetylase (HDAC) enzyme.

In some embodiments, the disease is a disease of the central nervous system. In some embodiments, the disease of the central nervous system comprises a neurodegenerative disease. In some embodiments, the disease of the central nervous system is depression. In some embodiments, the disease of the central nervous system is selected from the group consisting of schizophrenia, bipolar disorder, Alzheimer's disease, and Huntington's disease. In some embodiments, the disease of the central nervous system further comprises depression. In some embodiments, the disease of the central nervous system is associated with abnormal expression or abnormal activity of a histone deacetylase (HDAC) enzyme.

In some embodiments, the disease is an inflammatory autoimmune disease. In some embodiments, the inflammatory autoimmune disease is associated with abnormal expression or abnormal activity of a histone deacetylase (HDAC) enzyme.

In some embodiments, about 0.1% to about 5% of the compound administered crosses the blood brain barrier. In some embodiments, about 0.1% to about 4% of the compound administered crosses the blood brain barrier. In some embodiments, about 0.1% to about 2% of the compound administered crosses the blood brain barrier. In some embodiments, about 0.1% to about 1% of the compound administered crosses the blood brain barrier. In some embodiments, about 0.5% of the compound administered crosses the blood brain barrier.

In some embodiments, the compound administered has a brain:plasma ratio of at least about 1:1. In some embodiments, the compound administered has a brain:plasma ratio of at least about 2:1. In some embodiments, the compound administered has a brain:plasma ratio of at least about 3:1. In some embodiments, the compound administered has a brain:plasma ratio of at least about 4:1. In some embodiments, the compound administered has a brain:plasma ratio of at least about 5:1. In some embodiments, the compound has a brain:plasma ratio of at least about 10:1. In some embodiments, the compound has a brain:plasma ratio of at least about 15:1. In some embodiments, the compound has a brain:plasma ratio of at least about 20:1. In some embodiments, the compound has a brain:plasma ratio of at least about 30:1. In some embodiments, the compound has a brain:plasma ratio of at least about 50:1.

The present application further provides a method of treating a cancer in a patient, the method comprising:

i) identifying the cancer as being associated with abnormal activity or abnormal expression of a histone deacetylase (HDAC); and

ii) if the cancer is identified as being associated with abnormal activity of a histone deacetylase (HDAC), then administering to the patient a therapeutically effective amount of a compound of Formula (I), or a pharmaceutically acceptable salt thereof.

The present application further provides a method of treating a disease of the central nervous in a patient, the method comprising:

i) identifying the disease of the central nervous system as being associated with abnormal activity or abnormal expression of a histone deacetylase (HDAC); and

ii) if the disease of the central nervous system is identified as being associated with abnormal activity or abnormal expression of a histone deacetylase (HDAC), then administering to the patient a therapeutically effective amount of a compound of Formula (I), or a pharmaceutically acceptable salt thereof.

The present application further provides a method of treating an inflammatory autoimmune disease in a patient, the method comprising:

i) identifying the inflammatory autoimmune disease as being associated with abnormal activity or abnormal expression of a histone deacetylase (HDAC); and

ii) if the inflammatory autoimmune disease is identified as being associated with abnormal activity or abnormal expression of a histone deacetylase (HDAC), then administering to the patient a therapeutically effective amount of a compound of Formula (I), or a pharmaceutically acceptable salt thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1A shows summed Positron Emission Tomography (PET) images from 1 to 60 minutes following injection with the radiolabeled compound of Example 3 at baseline and after 5 minutes pretreatment with 0.5 mg/kg and 2.0 mg/kg of the unlabeled compound of Example 2.

FIG. 1B shows the percent uptake in whole brain of the radiolabeled compound of Example 3 from 5 to 50 minutes.

FIG. 1C shows the percent uptake in the whole brain of increasing concentrations of the radiolabeled compound of Example 3.

FIG. 2A shows the percent uptake in the whole brain of the radiolabeled compound of Example 3 in the presence of: (left to right) vehicle (5 minutes prior to radiotracer, mean±standard error, n=11); or the compound of Example 2 administered at 5.0 mg/kg, 2.5 and 7.5 h prior to radiotracer; 1.0 mg/kg, 2.5, 4, and 7.5 h prior to radiotracer; and 0.1 mg/kg, 4 h prior to radiotracer.

FIG. 2B shows the percent uptake in the whole brain of the radiolabeled compound of Example 3 in the presence of 5.0 mg/kg, 1.0 mg/kg, and 0.1 mg/kg of the compound of Example 2 at 2.5 h and 4 h.

FIG. 2C shows the percent uptake in the whole brain of the radiolabeled compound of Example 3 in the presence of 5.0 mg/kg, 1.0 mg/kg, and 0.1 mg/kg of the compound of Example 2 at 2.5 h, 4 h, and 7.5 h.

FIG. 3A shows immobility time data representative of a forced swim test in rats treated with the compound of Example 2 compared to a control.

FIG. 3B shows an immobility scatterplot representative of a forced swim test in rats treated with the compound of Example 2 compared to a control.

FIG. 4A shows the percent change in whole brain uptake via in vivo PET-imaging of the HDAC-selective radiolabeled compound of Example 3 representative of rats in the presence of vehicle or the compound of Example 2.

FIG. 4B shows the percentage of initial weight of as a function of treatment day representative of rats in the presence of vehicle or the compound of Example 2.

FIG. 5 shows the distance traveled over 10 minutes in an open field test upon treatment with the compound of Example 2 compared to a control.

FIG. 6 shows the pharmacokinetic parameters of the compound of Example 2 in plasma and brain following a single intraperitoneal administration to male Sprague Dawley rats.

FIG. 7 shows the brain-to-plasma exposure ratio following a single intraperitoneal administration of the compound of Example 2 to male Sprague Dawley rats.

FIG. 8 shows plasma concentration-time data following a single intraperitoneal administration of the compound of Example 2 in male Sprague Dawley rats.

FIG. 9 shows brain concentration-time data with 3-fold dilution following a single intraperitoneal administration of the compound of Example 2 in male Sprague Dawley rats.

FIG. 10 shows brain concentration-time data with following a single intraperitoneal administration of the compound of Example 2 in male Sprague Dawley rats.

FIG. 11A shows a mean plasma and brain concentration-time profile fit to a linear curve following a single intraperitoneal administration of the compound of Example 2 in male Sprague Dawley rats.

FIG. 11B shows a mean plasma and brain concentration-time profile fit to a logarithmic curve following a single intraperitoneal administration of the compound of Example 2 in male Sprague Dawley rats.

FIG. 12 shows the pharmacokinetic parameters of the compound of Example 2 in plasma and brain following a single intravenous administration to male Sprague Dawley rats.

FIG. 13 shows the brain-to-plasma exposure ratio following a single intravenous administration of the compound of Example 2 to male Sprague Dawley rats.

FIG. 14 shows plasma concentration-time data following a single intravenous administration of the compound of Example 2 in male Sprague Dawley rats.

FIG. 15 shows brain concentration (ng/mL)-time data following a single intravenous administration of the compound of Example 2 in male Sprague Dawley rats.

FIG. 16 shows brain concentration (ng/g)-time data with following a single intravenous administration of the compound of Example 2 in male Sprague Dawley rats.

FIG. 17A shows a mean plasma and brain concentration-time profile fit to a linear curve following a single intravenous administration of the compound of Example 2 in male Sprague Dawley rats.

FIG. 17B shows a mean plasma and brain concentration-time profile fit to a logarithmic curve following a single intravenous administration of the compound of Example 2 in male Sprague Dawley rats.

FIG. 18A shows the chromatographic and mass spectrometric conditions used in Example 11.

FIG. 18B shows the HPLC gradient conditions used in Example 11.

FIG. 19A shows the chromatographic and mass spectrometric conditions used in Example 12.

FIG. 19B shows the HPLC gradient conditions used in Example 12.

FIG. 20A shows a timeline schematic of blocking dose administration and PET/CT imaging. All animals were stabilized on anesthesia at least 20 min prior to radiotracer administration.

FIG. 20B shows the dynamic tracer uptake in whole brain, evaluated using ROI analysis on dynamic imaging datasets from n=9 ‘baseline’ rats blocked with vehicle (10% DMSO, 10% Tween 80, 80% saline) 5 min prior to tracer administration. Data are expressed as percent uptake in whole brain relative to uptake at time=600 sec (mean±standard deviation).

FIG. 20C shows self-blocking of radiotracer binding (total radioactivity accumulation in whole brain), demonstrated via pretreatment (5 min, i.v.) with a dose range (0.001 mg/kg-2.0 mg/kg) of unlabeled radiotracer and compared to baseline controls.

FIG. 20D shows quantification of [¹¹C] activity in whole brain at time 3600 s measured via trend in accumulated radioactivity from time=10 min to time=60 min for baseline (white bar, 0 mg/kg) and each blocking condition (grayscale).

FIG. 20E shows Spearman correlation with directional t-test, identifying dose-response relationships for relative [¹¹C] blockade in whole brain in rat (gray triangles, r=0.89; p=0.006) and whole-brain volume of distribution (V_{T}) from analogous PET experiments in non-human primate (NHP; white squares, r=0.90; p=0.042), with blocking doses scaled for NHP equivalent dose (mg/kg).

FIG. 20F shows pretreatment with unlabeled radiotracer at 5 min, 4 h or 24 h prior to tracer administration to demonstrate time-dependent (short-lasting) effects of self-blockade.

FIG. 21 shows the [¹¹C] percent uptake in the whole brain in the after pretreatment with the compound of Example 2.

### DETAILED DESCRIPTION

Inhibitors of histone deacetylase (HDAC) enzymes have been demonstrated to beneficially alter rodent behaviors in CNS-disease relevant paradigms, however, are limited in therapeutic efficacy given the poor brain penetrance (e.g., <1:10 brain to plasma ratio). Provided herein are HDAC inhibitors that exhibit improved brain penetrance and offer potential to function as therapeutics in a diverse range of CNS diseases including, but not limited to, mood and mental disorders, neurodegenerative diseases, disorders of learning, memory, and cognition.

It is appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

### Compounds

The present application provides, inter alia, a compound of Formula (I):

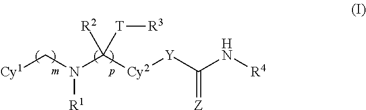

or a pharmaceutically acceptable salt thereof, wherein:

each T is independently absent or a C₁₋₄ alkylene;

Y is absent, C₁₋₄ alkylene, or C₂₋₄ alkenylene;

Z is selected from the group consisting of O, S, and NR^{b};

R¹ is selected from the group consisting of H, C₁₋₆ alkyl, C₁₋₆ haloalkyl, C₂₋₆ alkenyl, C₂₋₆ alkynyl, and OR^{a}; wherein said C₁₋₆ alkyl, C₂₋₆ alkenyl, and C₂₋₆ alkynyl are each optionally substituted with 1, 2, 3, or 4 independently selected R⁶ groups;

each R² is independently selected from the group consisting of absent, H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}; wherein said C₁₋₆ alkyl, C₂₋₆ alkenyl, and C₂₋₆ alkynyl are each optionally substituted with 1, 2, 3, or 4 independently selected R⁶ groups;

each R³ is independently selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, —S(═O)₂R^{c}, (═O), (═S), and (═NR^{b}); wherein said C₁₋₆ alkyl, C₂₋₆ alkenyl, and C₂₋₆ alkynyl are each optionally substituted with 1, 2, 3, or 4 independently selected R⁶ groups;

R⁴ is selected from the group consisting of H, C₁₋₆ alkyl, C₁₋₆ haloalkyl, C₂₋₆ alkenyl, C₂₋₆ alkynyl, OR^{a}, and Cy³; wherein said C₁₋₆ alkyl, C₂₋₆ alkenyl, and C₂₋₆ alkynyl are each optionally substituted with 1, 2, 3, or 4 independently selected R⁶ groups;

each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

each R^{a}, R^{b}, and R^{c} is independently H or C₁₋₆ alkyl;

Cy¹ is selected from the group consisting of C₃₋₁₀ cycloalkyl, 4-10 membered heterocycloalkyl, C₆₋₁₀ aryl, and 4-10 membered heteroaryl; wherein said C₃₋₁₀ cycloalkyl, 4-10 membered heterocycloalkyl, C₆₋₁₀ aryl, and 4-10 membered heteroaryl are each optionally substituted by 1, 2, 3, or 4 substituents independently selected from C₁₋₆ alkyl, C₁₋₆ haloalkyl, C₁₋₄ alkoxy, halo, OH, and CN;

Cy² is selected from the group consisting of C₆₋₁₀ aryl, C₃₋₁₀ cycloalkyl, 4-10 membered heteroaryl, and 4-10 membered heterocycloalkyl; wherein said C₆₋₁₀ aryl, C₃₋₁₀ cycloalkyl, 4-10 membered heteroaryl, and 4-10 membered heterocycloalkyl are each optionally substituted by 1, 2, 3, or 4 substituents independently selected from C₁₋₆ alkyl, C₁₋₆ haloalkyl, C₁₋₄ alkoxy, halo, OH, and CN;

Cy³ is selected from the group consisting of C₃₋₁₀ cycloalkyl, 4-10 membered heterocycloalkyl, C₆₋₁₀ aryl, and 4-10 membered heteroaryl; wherein said C₃₋₁₀ cycloalkyl, 4-10 membered heterocycloalkyl, C₆₋₁₀ aryl, and 4-10 membered heteroaryl are each substituted by 1, 2, 3, or 4 substituents independently selected from halo, OH, CN, C₁₋₆ alkyl, C₁₋₆ haloalkyl, C₁₋₄ alkoxy, C₆₋₁₀ aryl, 4-10 membered heteroaryl, amino, C₁₋₃ alkylamino, di(C₁₋₃ alkyl)amino, carbamyl, C₁₋₃ alkylcarbamyl, di(C₁₋₃ alkyl)carbamyl, C₁₋₃ alkylcarbonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

m is 0, 1, 2, 3, 4, 5, or 6; and

p is 0, 1, 2, 3, 4, 5, or 6.

In some embodiments, the compound of Formula (I) does not include a compound selected from the group consisting of:

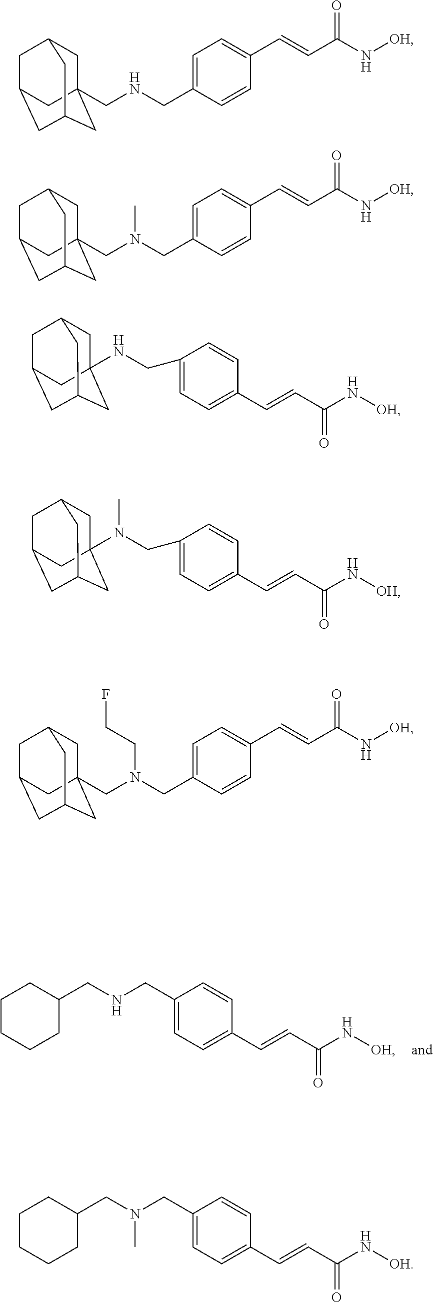

In some embodiments, m is 0, 1, or 2. In some embodiments, m is 0. In some embodiments, m is 1. In some embodiments, m is 2. In some embodiments, m is 0 or 1. In some embodiments, m is 0 or 2. In some embodiments, m is 1 or 2.

In some embodiments, p is 0, 1, or 2. In some embodiments, p is 0. In some embodiments, p is 1. In some embodiments, p is 2. In some embodiments, p is 0 or 1. In some embodiments, p is 0 or 2. In some embodiments, p is 1 or 2.

In some embodiments, m is 0, 1, or 2 and p is 1. In some embodiments m is 0 and p is 1. In some embodiments, m is 1, and p is 1. In some embodiments, m is 2 and p is 1.

In some embodiments, Y is absent or C₂₋₄ alkenylene. In some embodiments, Y is C₂₋₄ alkenylene. In some embodiments, Y is —CH═CH—. In some embodiments, Y is:

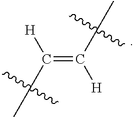 [SMILES: */C=C/*]

In some embodiments, Y is absent.

In some embodiments, Z is O.

In some embodiments, each T is an independently selected C₁₋₄ alkylene. In some embodiments, each T is absent. In some embodiments, each T is absent when each R³ is independently selected from the group consisting of (═O), (═S), and (═NR^{b}). In some embodiments, T cannot be absent when R³, together with the carbon atom to which T and R³ are connected, is selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}.

In some embodiments, R¹ is selected from the group consisting of H, C₁₋₆ alkyl, and C₁₋₆ haloalkyl. In some embodiments, R¹ is selected from the group consisting of H, —CH₃, and —CH₂CH₂F. In some embodiments, R¹ is H. In some embodiments, R¹ is —CH₃.

In some embodiments, R¹ is —CH₂CH₂F.

In some embodiments, each R² is independently selected from the group consisting of absent, H, C₁₋₆ alkyl, and C₁₋₆ haloalkyl. In some embodiments, each R² is independently selected from the group consisting of H, C₁₋₆ alkyl, and C₁₋₆ haloalkyl. In some embodiments, each R² is H. In some embodiments, each R² is absent when each R³ is independently selected from the group consisting of (═O), (═S), and (═NR^{b}). In some embodiments, R² is absent when R³ is independently selected from the group consisting of (═O), (═S), and (═NR^{b}). In some embodiments, R² cannot be absent when R³, together with the carbon atom to which R² and R³ are connected, is selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}.

In some embodiments, each R³ is independently selected from the group consisting of H, C₁₋₆ alkyl, C₁₋₆ haloalkyl, OR^{a}, (═O), (═S), and (═NR^{b}). In some embodiments, each R³ is independently selected from the group consisting of H, OR^{a}, and (═O). In some embodiments, each R³ is independently H or (═O). In some embodiments, each R³ is H. In some embodiments, each R³ is (═O). In some embodiments, when each R³ is independently selected from the group consisting of (═O), (═S) and (NR^{b}), then each R² is absent. In some embodiments, when each R³ is independently selected from the group consisting of (═O), (═S) and (NR^{b}), then each T is absent. In some embodiments, when each R³ is independently selected from the group consisting of (═O), (═S) and (NR^{b}), then each R² is absent and each T is absent. In some embodiments, each R³ is (═O) and each R² is absent. In some embodiments, each R³ is (═O) and each T is absent. In some embodiments, each R³ is (═O), each R² is absent, and each T is absent.

In some embodiments, R⁴ is selected from the group consisting of H, OH, or Cy³.

In some embodiments, R⁴ is OH or Cy³. In some embodiments, R⁴ is Cy³. In some embodiments, R⁴ is Cy³, and Cy³ is C₆₋₁₀ aryl or a 4-10 membered heteroaryl ring. In some embodiments, R⁴ is Cy³, and Cy³ is C₆₋₁₀ aryl. In some embodiments, R⁴ is Cy³, and Cy³ is phenyl. In some embodiments, R⁴ is Cy³, and Cy³ is phenyl, wherein the phenyl is substituted by 1, 2, 3, or 4 substituents independently selected from halo, OH, CN, C₁₋₆ alkyl, C₁₋₆ haloalkyl, C₁₋₄ alkoxy, C₆₋₁₀ aryl, 4-10 membered heteroaryl, amino, C₁₋₃ alkylamino, and di(C₁₋₃ alkyl)amino. In some embodiments, R⁴ is Cy³, and Cy³ is phenyl, wherein the phenyl is substituted by 1, 2, 3, or 4 substituents independently selected from amino, C₁₋₃ alkylamino, and di(C₁₋₃ alkyl)amino. In some embodiments, R⁴ is Cy³, and Cy³ is phenyl, wherein the phenyl is substituted by 1 amino group. In some embodiments, R⁴ is Cy³, and Cy³ is:

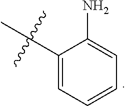 [SMILES: *c1ccccc1N]

In some embodiments, R⁴ is Cy³, and Cy³ is phenyl, wherein the phenyl is substituted by 1 or 2 substituents independently selected from the group consisting of halo, OH, CN, C₁₋₆ alkyl, C₁₋₆ haloalkyl, C₁₋₄ alkoxy, C₆₋₁₀ aryl, 4-10 membered heteroaryl, and amino. In some embodiments, R⁴ is Cy³, and Cy³ is phenyl, wherein the phenyl is substituted by 2 substituents independently selected from the group consisting of halo, OH, CN, C₁₋₆ alkyl, C₁₋₆ haloalkyl, C₁₋₄ alkoxy, C₆₋₁₀ aryl, 4-10 membered heteroaryl, and amino. In some embodiments, R⁴ is Cy³, and Cy³ is phenyl, wherein the phenyl is substituted by 2 substituents independently selected from the group consisting of halo, C₆₋₁₀ aryl, 4-10 membered heteroaryl, and amino. In some embodiments, R⁴ is Cy³, and Cy³ is phenyl, wherein the phenyl is substituted by 2 substituents independently selected from the group consisting of fluoro, phenyl, thiophene, and amino. In some embodiments, R⁴ is selected from the group consisting of:

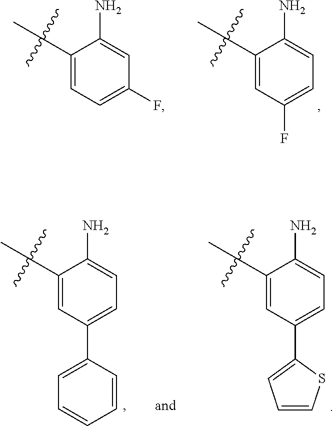 [SMILES: *c1ccc(F)cc1N ; *c1cc(F)ccc1N ; *c1cc(-c2ccccc2)ccc1N ; *c1cc(-c2cccs2)ccc1N]

In some embodiments, R⁴ is OH.

In some embodiments, each R⁶ is independently selected from the group consisting of OH, NO₂, CN, halo, and C₁₋₃ alkyl.

In some embodiments, Cy¹ is a C₃₋₁₀ cycloalkyl or a 4-10 membered heterocycloalkyl. In some embodiments, Cy¹ is C₃₋₁₀ cycloalkyl. In some embodiments, Cy¹ is cyclohexyl or adamantyl. In some embodiments, Cy¹ is cyclohexyl. In some embodiments, Cy¹ is adamantyl.

In some embodiments, Cy² is selected from the group consisting of C₆-10 aryl, 4-10 membered heteroaryl, and 4-10 membered heterocycloalkyl. In some embodiments, Cy² is C₆₋₁₀ aryl or 4-10 membered heterocycloalkyl. In some embodiments, Cy² is a 4-10 membered heterocycloalkyl group. In some embodiments, Cy² is azetidinyl. In some embodiments, Cy² is C₆₋₁₀ aryl. In some embodiments, Cy² is phenyl.

In some embodiments, each T is independently absent or —CH₂—; Y is C₂₋₄ alkenylene; Z is O; R¹ is selected from the group consisting of H, C₁₋₆ alkyl, and C₁₋₆ haloalkyl; each R² is independently H or absent; each R³ is independently H or (═O); R⁴ is OH or Cy³; Cy¹ is C₃₋₁₀ cycloalkyl; Cy² is C₆₋₁₀ aryl or 4-10 membered heterocycloalkyl; Cy³ is C₆₋₁₀ aryl; m is 0, 1, or 2; and p is 1.

In some embodiments, T is independently absent or —CH₂—; Y is —CH═CH—; Z is O; R¹ is selected from the group consisting of H, —CH₃, and —CH₂CH₂F; each R² is independently H or absent; each R³ is independently H or (═O); R⁴ is OH or Cy³; Cy¹ is cyclohexyl or adamantyl; Cy² is phenyl or azetidinyl; Cy³ is phenyl, wherein the phenyl is substituted by 1 amino group; m is 0, 1, or 2; and p is 1.

In some embodiments, T and R² are each absent; Y is —CH═CH—; Z is O; R¹ is selected H; R³ is (═O); R⁴ is OH or Cy³; Cy¹ is C₃₋₁₀ cycloalkyl; Cy² is phenyl or azetidinyl; Cy³ is phenyl, wherein the phenyl is substituted by 1 amino group; m is 1 or 2; and p is 1.

In some embodiments, T is absent; Y is —CH═CH—; R¹ is selected from the group consisting of H, —CH₃, and —CH₂CH₂F; R² is H; R³ is H; R⁴ is OH or Cy³; Cy¹ is C₃₋₁₀ cycloalkyl; Cy² is C₆₋₁₀ aryl or 4-10 membered heterocycloalkyl; Cy³ is phenyl, wherein the phenyl is substituted by 1 amino group; m is 0 or 1; and p is 1.

In some embodiments, T is absent; Y is —CH═CH—; R¹ is selected from the group consisting of H, —CH₃, and —CH₂CH₂F; R² is H; R³ is H; R⁴ is OH or Cy³; Cy¹ is cyclohexyl or adamantyl; Cy² is phenyl or azetidinyl; Cy³ is phenyl, wherein the phenyl is substituted by 1 amino group; m is 0 or 1; and p is 1.

In some embodiments, Cy¹ is cyclohexyl or adamantyl; Cy² is phenyl or azetidinyl; R⁴ is Cy³; and Cy³ is phenyl, wherein the phenyl is substituted by 1 amino group.

In some embodiments, Cy¹ is cyclohexyl or adamantyl; Cy² is phenyl or azetidinyl; and R⁴ is OH.

In some embodiments, Cy¹ is adamantyl; Cy² is phenyl or azetidinyl; R⁴ is Cy³; and Cy³ is phenyl, wherein the phenyl is substituted by 2 substituents independently selected from the group consisting of halo, C₆₋₁₀ aryl, 4-10 membered heteroaryl, and amino.

In some embodiments, the compound of Formula (I) is a compound of Formula (Ia):

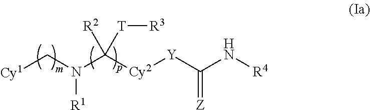

or a pharmaceutically acceptable salt thereof, wherein:

each T is independently absent or a C₁₋₄ alkylene;

Y is absent, C₁₋₄ alkylene, or C₂₋₄ alkenylene;

Z is selected from the group consisting of O, S, and NR^{b};

R¹ is selected from the group consisting of H, C₁₋₆ alkyl, C₁₋₆ haloalkyl, C₂₋₆ alkenyl, C₂₋₆ alkynyl, and OR^{a}; wherein the C₁₋₆ alkyl, C₂₋₆ alkenyl, and C₂₋₆ alkynyl are each optionally substituted with 1, 2, 3, or 4 independently selected R⁶ groups;

each R² is independently selected from the group consisting of absent, H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}; wherein the C₁₋₆ alkyl, C₂₋₆ alkenyl, and C₂₋₆ alkynyl are each optionally substituted with 1, 2, 3, or 4 independently selected R⁶ groups;

each R³ is independently selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, —S(═O)₂R^{c}, (═O), (═S), and (═NR^{b}); wherein the C₁₋₆ alkyl, C₂₋₆ alkenyl, and C₂₋₆ alkynyl are each optionally substituted with 1, 2, 3, or 4 independently selected R⁶ groups;

R⁴ is selected from the group consisting of H, C₁₋₆ alkyl, C₁₋₆ haloalkyl, C₂₋₆ alkenyl, C₂₋₆ alkynyl, OR^{a}, and Cy³; wherein the C₁₋₆ alkyl, C₂₋₆ alkenyl, and C₂₋₆ alkynyl are each optionally substituted with 1, 2, 3, or 4 independently selected R⁶ groups;

each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

each R^{a}, R^{b}, and R^{c} is independently H or C₁₋₆ alkyl;

Cy¹ is selected from the group consisting of C₃₋₁₀ cycloalkyl, 4-10 membered heterocycloalkyl, C₆₋₁₀ aryl, and 4-10 membered heteroaryl; wherein the C₃₋₁₀ cycloalkyl, 4-10 membered heterocycloalkyl, C₆₋₁₀ aryl, and 4-10 membered heteroaryl are each optionally substituted by 1, 2, 3, or 4 substituents independently selected from C₁₋₆ alkyl, C₁₋₆ haloalkyl, C₁₋₄ alkoxy, halo, OH, and CN;

Cy² is C₆₋₁₀ aryl or 4-10 membered heteroaryl; wherein the C₆₋₁₀ aryl and 4-10 membered heteroaryl are each optionally substituted by 1, 2, 3, or 4 substituents independently selected from C₁₋₆ alkyl, C₁₋₆ haloalkyl, C₁₋₄ alkoxy, halo, OH, and CN;

Cy³ is selected from the group consisting of C₃₋₁₀ cycloalkyl, 4-10 membered heterocycloalkyl, C₆₋₁₀ aryl, and 4-10 membered heteroaryl; wherein the C₃₋₁₀ cycloalkyl, 4-10 membered heterocycloalkyl, C₆₋₁₀ aryl, and 4-10 membered heteroaryl are each substituted by 1, 2, 3, or 4 substituents independently selected from halo, OH, CN, C₁₋₆ alkyl, C₁₋₆ haloalkyl, C₁₋₄ alkoxy, C₆₋₁₀ aryl, 4-10 membered heteroaryl, amino, C₁₋₃ alkylamino, di(C₁₋₃ alkyl)amino, carbamyl, C₁₋₃ alkylcarbamyl, di(C₁₋₃ alkyl)carbamyl, C₁₋₃ alkylcarbonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

m is 0, 1, 2, 3, 4, 5, or 6; and

p is 0, 1, 2, 3, 4, 5, or 6.

In some embodiments, the compound of Formula (Ia) does not include a compound selected from the group consisting of:

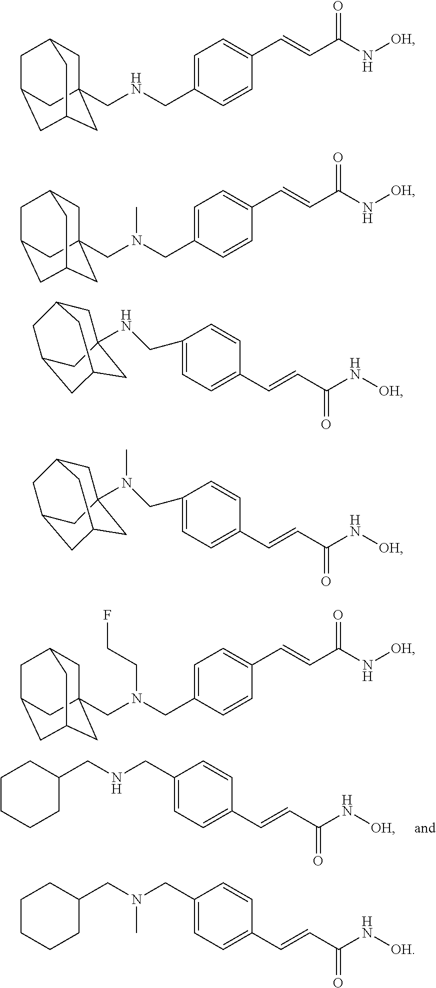

In some embodiments, m is 0, 1, or 2. In some embodiments, m is 0. In some embodiments, m is 1. In some embodiments, m is 2. In some embodiments, m is 0 or 1. In some embodiments, m is 0 or 2. In some embodiments, m is 1 or 2.

In some embodiments, p is 0, 1, or 2. In some embodiments, p is 0. In some embodiments, p is 1. In some embodiments, p is 2. In some embodiments, p is 0 or 1. In some embodiments, p is 0 or 2. In some embodiments, p is 1 or 2.

In some embodiments, m is 0, 1, or 2 and p is 1. In some embodiments m is 0 and p is 1. In some embodiments, m is 1, and p is 1. In some embodiments, m is 2 and p is 1.

In some embodiments, Y is absent or C₂₋₄ alkenylene. In some embodiments, Y is C₂₋₄ alkenylene. In some embodiments, Y is —CH═CH—. In some embodiments, Y is:

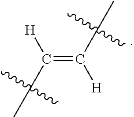 [SMILES: */C=C/*]

In some embodiments, Y is absent.

In some embodiments, Z is O.

In some embodiments, each T is an independently selected C₁₋₄ alkylene. In some embodiments, each T is absent. In some embodiments, each T is absent when each R³ is independently selected from the group consisting of (═O), (═S), and (═NR^{b}). In some embodiments, T cannot be absent when R³, together with the carbon atom to which T and R³ are connected, is selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}.

In some embodiments, R¹ is selected from the group consisting of H, C₁₋₆ alkyl, and C₁₋₆ haloalkyl. In some embodiments, R¹ is selected from the group consisting of H, —CH₃, and —CH₂CH₂F. In some embodiments, R¹ is H. In some embodiments, R¹ is —CH₃.

In some embodiments, R¹ is —CH₂CH₂F.

In some embodiments, each R² is independently selected from the group consisting of absent, H, C₁₋₆ alkyl, and C₁₋₆ haloalkyl. In some embodiments, each R² is independently selected from the group consisting of H, C₁₋₆ alkyl, and C₁₋₆ haloalkyl. In some embodiments, each R² is H. In some embodiments, each R² is absent when each R³ is independently selected from the group consisting of (═O), (═S), and (═NR^{b}). In some embodiments, R² is absent when R³ is independently selected from the group consisting of (═O), (═S), and (═NR^{b}). In some embodiments, R² cannot be absent when R³, together with the carbon atom to which R² and R³ are connected, is selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R, and —S(═O)₂R^{c}.

In some embodiments, each R³ is independently selected from the group consisting of H, C₁₋₆ alkyl, C₁₋₆ haloalkyl, OR^{a}, (═O), (═S), and (═NR^{b}). In some embodiments, each R³ is independently selected from the group consisting of H, OR^{a}, and (═O). In some embodiments, each R³ is independently H or (═O). In some embodiments, each R³ is H. In some embodiments, each R³ is (═O). In some embodiments, when each R³ is independently selected from the group consisting of (═O), (═S) and (NR^{b}), then each R² is absent. In some embodiments, when each R³ is independently selected from the group consisting of (═O), (═S) and (NR^{b}), then each T is absent. In some embodiments, when each R³ is independently selected from the group consisting of (═O), (═S) and (NR^{b}), then each R² is absent and each T is absent. In some embodiments, each R³ is (═O) and each R² is absent. In some embodiments, each R³ is (═O) and each T is absent. In some embodiments, each R³ is (═O), each R² is absent, and each T is absent.

In some embodiments, R⁴ is selected from the group consisting of H, OH, or Cy³.

In some embodiments, R⁴ is OH or Cy³. In some embodiments, R⁴ is Cy³. In some embodiments, R⁴ is Cy³, and Cy³ is C₆₋₁₀ aryl or a 4-10 membered heteroaryl ring. In some embodiments, R⁴ is Cy³, and Cy³ is C₆₋₁₀ aryl. In some embodiments, R⁴ is Cy³, and Cy³ is phenyl. In some embodiments, R⁴ is Cy³, and Cy³ is phenyl, wherein the phenyl is substituted by 1, 2, 3, or 4 substituents independently selected from halo, OH, CN, C₁₋₆ alkyl, C₁₋₆ haloalkyl, C₁₋₄ alkoxy, C₆₋₁₀ aryl, 4-10 membered heteroaryl, amino, C₁₋₃ alkylamino, and di(C₁₋₃ alkyl)amino. In some embodiments, R⁴ is Cy³, and Cy³ is phenyl, wherein the phenyl is substituted by 1, 2, 3, or 4 substituents independently selected from amino, C₁₋₃ alkylamino, and di(C₁₋₃ alkyl)amino. In some embodiments, R⁴ is Cy³, and Cy³ is phenyl, wherein the phenyl is substituted by 1 amino group. In some embodiments, R⁴ is Cy³, and Cy³ is:

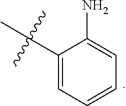 [SMILES: *c1ccccc1N]

In some embodiments, R⁴ is Cy³, and Cy³ is phenyl, wherein the phenyl is substituted by 1 or 2 substituents independently selected from the group consisting of halo, OH, CN, C₁₋₆ alkyl, C₁₋₆ haloalkyl, C₁₋₄ alkoxy, C₆₋₁₀ aryl, 4-10 membered heteroaryl, and amino. In some embodiments, R⁴ is Cy³, and Cy³ is phenyl, wherein the phenyl is substituted by 2 substituents independently selected from the group consisting of halo, OH, CN, C₁₋₆ alkyl, C₁₋₆ haloalkyl, C₁₋₄ alkoxy, C₆₋₁₀ aryl, 4-10 membered heteroaryl, and amino. In some embodiments, R⁴ is Cy³, and Cy³ is phenyl, wherein the phenyl is substituted by 2 substituents independently selected from the group consisting of halo, C₆₋₁₀ aryl, 4-10 membered heteroaryl, and amino. In some embodiments, R⁴ is Cy³, and Cy³ is phenyl, wherein the phenyl is substituted by 2 substituents independently selected from the group consisting of fluoro, phenyl, thiophene, and amino. In some embodiments, R⁴ is selected from the group consisting of:

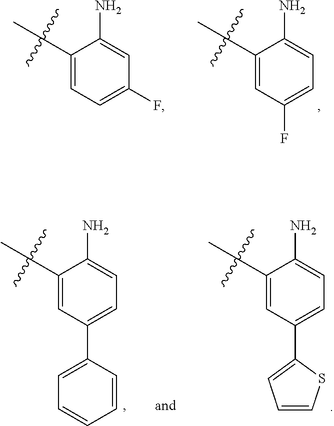 [SMILES: *c1ccc(F)cc1N ; *c1cc(F)ccc1N ; *c1cc(-c2ccccc2)ccc1N ; *c1cc(-c2cccs2)ccc1N]

In some embodiments, R⁴ is OH.

In some embodiments, each R⁶ is independently selected from the group consisting of OH, NO₂, CN, halo, and C₁₋₃ alkyl.

In some embodiments, Cy¹ is a C₃₋₁₀ cycloalkyl or a 4-10 membered heterocycloalkyl. In some embodiments, Cy¹ is C₃₋₁₀ cycloalkyl. In some embodiments, Cy¹ is cyclohexyl or adamantyl. In some embodiments, Cy¹ is cyclohexyl. In some embodiments, Cy¹ is adamantyl.

In some embodiments, Cy² is C₆₋₁₀ aryl. In some embodiments, Cy² is phenyl.

In some embodiments, each T is independently absent or —CH₂—; Y is C₂₋₄ alkenylene; Z is O; R¹ is selected from the group consisting of H, C₁₋₆ alkyl, and C₁₋₆ haloalkyl; each R² is independently H or absent; each R³ is independently H or (═O); R⁴ is OH or Cy³; Cy¹ is C₃₋₁₀ cycloalkyl; Cy² is C₆₋₁₀ aryl; Cy³ is C₆₋₁₀ aryl; m is 0, 1, or 2; and p is 1.

In some embodiments, T is independently absent or —CH₂—; Y is —CH═CH—; Z is O; R¹ is selected from the group consisting of H, —CH₃, and —CH₂CH₂F; each R² is independently H or absent; each R³ is independently H or (═O); R⁴ is OH or Cy³; Cy¹ is cyclohexyl or adamantyl; Cy² is phenyl; Cy³ is phenyl, wherein the phenyl is substituted by 1 amino group; m is 0, 1, or 2; and p is 1.

In some embodiments, T and R² are each absent; Y is —CH═CH—; Z is O; R¹ is selected H; R³ is (═O); R⁴ is OH or Cy³; Cy¹ is C₃₋₁₀ cycloalkyl; Cy² is phenyl; Cy³ is phenyl, wherein the phenyl is substituted by 1 amino group; m is 1 or 2; and p is 1.

In some embodiments, T is absent; Y is —CH═CH—; R¹ is selected from the group consisting of H, —CH₃, and —CH₂CH₂F; R² is H; R³ is H; R⁴ is OH or Cy³; Cy¹ is C₃₋₁₀ cycloalkyl; Cy² is C₆₋₁₀ aryl; Cy³ is phenyl, wherein the phenyl is substituted by 1 amino group; m is 0 or 1; and p is 1.

In some embodiments, T is absent; Y is —CH═CH—; R¹ is selected from the group consisting of H, —CH₃, and —CH₂CH₂F; R² is H; R³ is H; R⁴ is OH or Cy³; Cy¹ is cyclohexyl or adamantyl; Cy² is phenyl; Cy³ is phenyl, wherein the phenyl is substituted by 1 amino group; m is 0 or 1; and p is 1.

In some embodiments, Cy¹ is cyclohexyl or adamantyl; Cy² is phenyl; R⁴ is Cy³; and Cy³ is phenyl, wherein the phenyl is substituted by 1 amino group.

In some embodiments, Cy¹ is cyclohexyl or adamantyl; Cy² is phenyl; and R⁴ is OH.

In some embodiments, Cy¹ is adamantyl; Cy² is phenyl; R⁴ is Cy³; and Cy³ is phenyl, wherein the phenyl is substituted by 2 substituents independently selected from the group consisting of halo, C₆₋₁₀ aryl, 4-10 membered heteroaryl, and amino.

In some embodiments, the compound of Formula (I) or the compound of Formula (Ia) is a compound of Formula (II):

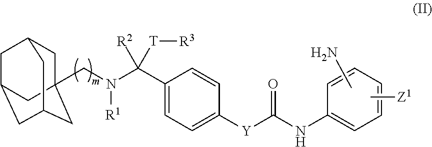

or a pharmaceutically acceptable salt thereof, wherein

T is absent or a C₁₋₄ alkylene;

Y is absent, C₁₋₄ alkylene, or C₂₋₄ alkenylene;

Z¹ is absent or selected from the group consisting of halo, OH, CN, C₁₋₆ alkyl, C₁₋₆ haloalkyl, C₁₋₄ alkoxy, C₆₋₁₀ aryl, and 4-10 membered heteroaryl;

R¹ is selected from the group consisting of H, C₁₋₆ alkyl, C₁₋₆ haloalkyl, C₂₋₆ alkenyl, C₂₋₆ alkynyl, and OR^{a}; wherein the C₁₋₆ alkyl, C₂₋₆ alkenyl, and C₂₋₆ alkynyl are each optionally substituted with 1, 2, 3, or 4 independently selected R⁶ groups;

R² is selected from the group consisting of absent, H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}; wherein the C₁₋₆ alkyl, C₂₋₆ alkenyl, and C₂₋₆ alkynyl are each optionally substituted with 1, 2, 3, or 4 independently selected R⁶ groups;

R³ is selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, —S(═O)₂R^{c}, (═O), (═S), and (═NR^{b}); wherein the C₁₋₆ alkyl, C₂₋₆ alkenyl, and C₂₋₆ alkynyl are each optionally substituted with 1, 2, 3, or 4 independently selected R⁶ groups;

each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

each R^{a}, R^{b}, and R^{c} is independently H or C₁₋₆ alkyl; and

m is 0, 1, 2, 3, 4, 5, or 6.

In some embodiments, the compound of Formula (I) or the compound of Formula (Ia) is a compound of Formula (III):

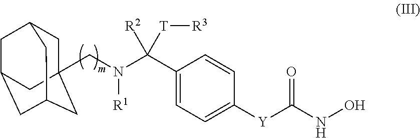

or a pharmaceutically acceptable salt thereof, wherein

T is absent or a C₁₋₄ alkylene;

Y is absent, C₁₋₄ alkylene, or C₂₋₄ alkenylene;

R¹ is selected from the group consisting of H, C₁₋₆ alkyl, C₁₋₆ haloalkyl, C₂₋₆ alkenyl, C₂₋₆ alkynyl, and OR^{a}; wherein the C₁₋₆ alkyl, C₂₋₆ alkenyl, and C₂₋₆ alkynyl are each optionally substituted with 1, 2, 3, or 4 independently selected R⁶ groups;

R² is selected from the group consisting of absent, H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}; wherein the C₁₋₆ alkyl, C₂₋₆ alkenyl, and C₂₋₆ alkynyl are each optionally substituted with 1, 2, 3, or 4 independently selected R⁶ groups;

R³ is selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, —S(═O)₂R^{c}, (═O), (═S), and (═NR^{b}); wherein the C₁₋₆ alkyl, C₂₋₆ alkenyl, and C₂₋₆ alkynyl are each optionally substituted with 1, 2, 3, or 4 independently selected R⁶ groups;

each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

each R^{a}, R^{b}, and R^{c} is independently H or C₁₋₆ alkyl; and

m is 0, 1, 2, 3, 4, 5, or 6.

In some embodiments, the compound of Formula (III) does not include a compound selected from the group consisting of:

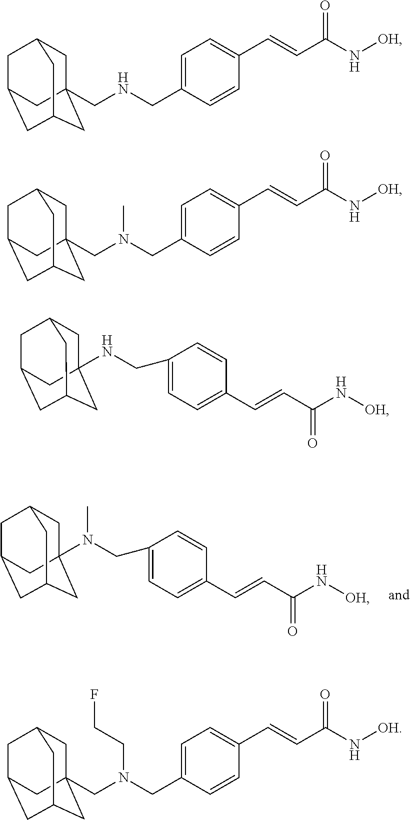

In some embodiments, the compound of Formula (I) or the compound of Formula (Ia) is a compound of Formula (IV):

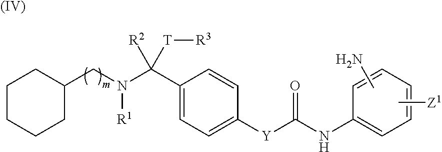

or a pharmaceutically acceptable salt thereof, wherein

T is absent or a C₁₋₄ alkylene;

Y is absent, C₁₋₄ alkylene, or C₂₋₄ alkenylene;

Z¹ is absent or selected from the group consisting of halo, OH, CN, C₁₋₆ alkyl, C₁₋₆ haloalkyl, C₁₋₄ alkoxy, C₆₋₁₀ aryl, and 4-10 membered heteroaryl;

R¹ is selected from the group consisting of H, C₁₋₆ alkyl, C₁₋₆ haloalkyl, C₂₋₆ alkenyl, C₂₋₆ alkynyl, and OR^{a}; wherein the C₁₋₆ alkyl, C₂₋₆ alkenyl, and C₂₋₆ alkynyl are each optionally substituted with 1, 2, 3, or 4 independently selected R⁶ groups;

R² is selected from the group consisting of absent, H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}; wherein the C₁₋₆ alkyl, C₂₋₆ alkenyl, and C₂₋₆ alkynyl are each optionally substituted with 1, 2, 3, or 4 independently selected R⁶ groups;

R³ is selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, —S(═O)₂R^{c}, (═O), (═S), and (═NR^{b}); wherein the C₁₋₆ alkyl, C₂₋₆ alkenyl, and C₂₋₆ alkynyl are each optionally substituted with 1, 2, 3, or 4 independently selected R⁶ groups;

each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

each R^{a}, R^{b}, and R^{c} is independently H or C₁₋₆ alkyl; and

m is 0, 1, 2, 3, 4, 5, or 6.

In some embodiments, the compound of Formula (I) or the compound of Formula (Ia) is a compound of Formula (V):

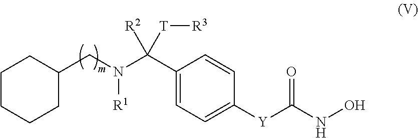

or a pharmaceutically acceptable salt thereof, wherein

T is absent or a C₁₋₄ alkylene;

Y is absent, C₁₋₄ alkylene, or C₂₋₄ alkenylene;

R¹ is selected from the group consisting of H, C₁₋₆ alkyl, C₁₋₆ haloalkyl, C₂₋₆ alkenyl, C₂₋₆ alkynyl, and OR^{a}; wherein the C₁₋₆ alkyl, C₂₋₆ alkenyl, and C₂₋₆ alkynyl are each optionally substituted with 1, 2, 3, or 4 independently selected R⁶ groups;

R² is selected from the group consisting of absent, H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}; wherein the C₁₋₆ alkyl, C₂₋₆ alkenyl, and C₂₋₆ alkynyl are each optionally substituted with 1, 2, 3, or 4 independently selected R⁶ groups;

R³ is selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, —S(═O)₂R^{c}, (═O), (═S), and (═NR^{b}); wherein the C₁₋₆ alkyl, C₂₋₆ alkenyl, and C₂₋₆ alkynyl are each optionally substituted with 1, 2, 3, or 4 independently selected R⁶ groups;

each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

each R^{a}, R^{b}, and R^{c} is independently H or C₁₋₆ alkyl; and

m is 0, 1, 2, 3, 4, 5, or 6.

In some embodiments, the compound of Formula (V) does not include a compound selected from the group consisting of:

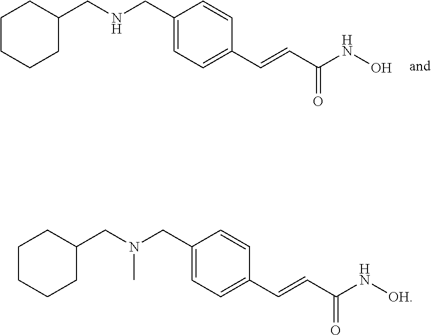 [SMILES: O=C(/C=C/c1ccc(CNCC2CCCCC2)cc1)NO ; CN(Cc1ccc(/C=C/C(=O)NO)cc1)CC1CCCCC1]

In some embodiments, the compound of Formula (I) is a compound of Formula (VI):

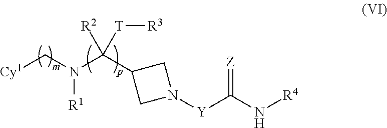

or a pharmaceutically acceptable salt thereof, wherein:

each T is independently absent or a C₁₋₄ alkylene;

Y is absent, C₁₋₄ alkylene, or C₂₋₄ alkenylene;

Z is selected from the group consisting of O, S, and NR^{b};

R¹ is selected from the group consisting of H, C₁₋₆ alkyl, C₁₋₆ haloalkyl, C₂₋₆ alkenyl, C₂₋₆ alkynyl, and OR^{a}; wherein said C₁₋₆ alkyl, C₂₋₆ alkenyl, and C₂₋₆ alkynyl are each optionally substituted with 1, 2, 3, or 4 independently selected R⁶ groups;

each R² is independently selected from the group consisting of absent, H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}; wherein said C₁₋₆ alkyl, C₂₋₆ alkenyl, and C₂₋₆ alkynyl are each optionally substituted with 1, 2, 3, or 4 independently selected R⁶ groups;

each R³ is independently selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, —S(═O)₂R^{c}, (═O), (═S), and (═NR^{b}); wherein said C₁₋₆ alkyl, C₂₋₆ alkenyl, and C₂₋₆ alkynyl are each optionally substituted with 1, 2, 3, or 4 independently selected R⁶ groups;

R⁴ is selected from the group consisting of H, C₁₋₆ alkyl, C₁₋₆ haloalkyl, C₂₋₆ alkenyl, C₂₋₆ alkynyl, OR^{a}, and Cy³; wherein said C₁₋₆ alkyl, C₂₋₆ alkenyl, and C₂₋₆ alkynyl are each optionally substituted with 1, 2, 3, or 4 independently selected R⁶ groups;

each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

each R^{a}, R^{b}, and R^{c} is independently H or C₁₋₆ alkyl;

Cy¹ is selected from the group consisting of C₃₋₁₀ cycloalkyl, 4-10 membered heterocycloalkyl, C₆₋₁₀ aryl, and 4-10 membered heteroaryl; wherein said C₃₋₁₀ cycloalkyl, 4-10 membered heterocycloalkyl, C₆₋₁₀ aryl, and 4-10 membered heteroaryl are each optionally substituted by 1, 2, 3, or 4 substituents independently selected from C₁₋₆ alkyl, C₁₋₆ haloalkyl, C₁₋₄ alkoxy, halo, OH, and CN;

Cy³ is selected from the group consisting of C₃₋₁₀ cycloalkyl, 4-10 membered heterocycloalkyl, C₆₋₁₀ aryl, and 4-10 membered heteroaryl; wherein said C₃₋₁₀ cycloalkyl, 4-10 membered heterocycloalkyl, C₆₋₁₀ aryl, and 4-10 membered heteroaryl are each substituted by 1, 2, 3, or 4 substituents independently selected from halo, OH, CN, C₁₋₆ alkyl, C₁₋₆ haloalkyl, C₁₋₄ alkoxy, C₆₋₁₀ aryl, 4-10 membered heteroaryl, amino, C₁₋₃ alkylamino, di(C₁₋₃ alkyl)amino, carbamyl, C₁₋₃ alkylcarbamyl, di(C₁₋₃ alkyl)carbamyl, C₁₋₃ alkylcarbonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

m is 0, 1, 2, 3, 4, 5, or 6; and

p is 0, 1, 2, 3, 4, 5, or 6.

In some embodiments, the compound of Formula (I) is a compound of Formula (VII):

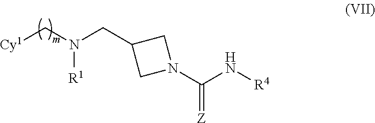

or a pharmaceutically acceptable salt thereof, wherein:

Z is selected from the group consisting of O, S, and NR^{b};

R¹ is selected from the group consisting of H, C₁₋₆ alkyl, C₁₋₆ haloalkyl, C₂₋₆ alkenyl, C₂₋₆ alkynyl, and OR^{a}; wherein said C₁₋₆ alkyl, C₂₋₆ alkenyl, and C₂₋₆ alkynyl are each optionally substituted with 1, 2, 3, or 4 independently selected R⁶ groups;

R⁴ is selected from the group consisting of H, C₁₋₆ alkyl, C₁₋₆ haloalkyl, C₂₋₆ alkenyl, C₂₋₆ alkynyl, OR^{a}, and Cy³; wherein said C₁₋₆ alkyl, C₂₋₆ alkenyl, and C₂₋₆ alkynyl are each optionally substituted with 1, 2, 3, or 4 independently selected R⁶ groups;

each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;

each R^{a} and R^{b} is independently H or C₁₋₆ alkyl;

Cy¹ is selected from the group consisting of C₃₋₁₀ cycloalkyl, 4-10 membered heterocycloalkyl, C₆₋₁₀ aryl, and 4-10 membered heteroaryl; wherein said C₃₋₁₀ cycloalkyl, 4-10 membered heterocycloalkyl, C₆₋₁₀ aryl, and 4-10 membered heteroaryl are each optionally substituted by 1, 2, 3, or 4 substituents independently selected from C₁₋₆ alkyl, C₁₋₆ haloalkyl, C₁₋₄ alkoxy, halo, OH, and CN;

Cy³ is selected from the group consisting of C₃₋₁₀ cycloalkyl, 4-10 membered heterocycloalkyl, C₆₋₁₀ aryl, and 4-10 membered heteroaryl; wherein said C₃₋₁₀ cycloalkyl, 4-10 membered heterocycloalkyl, C₆₋₁₀ aryl, and 4-10 membered heteroaryl are each substituted by 1, 2, 3, or 4 substituents independently selected from halo, OH, CN, C₁₋₆ alkyl, C₁₋₆ haloalkyl, C₁₋₄ alkoxy, C₆₋₁₀ aryl, 4-10 membered heteroaryl, amino, C₁₋₃ alkylamino, di(C₁₋₃ alkyl)amino, carbamyl, C₁₋₃ alkylcarbamyl, di(C₁₋₃ alkyl)carbamyl, C₁₋₃ alkylcarbonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino; and

m is 0, 1, 2, 3, 4, 5, or 6.

In some embodiments, the compound of Formula (I) is selected from the group consisting of:

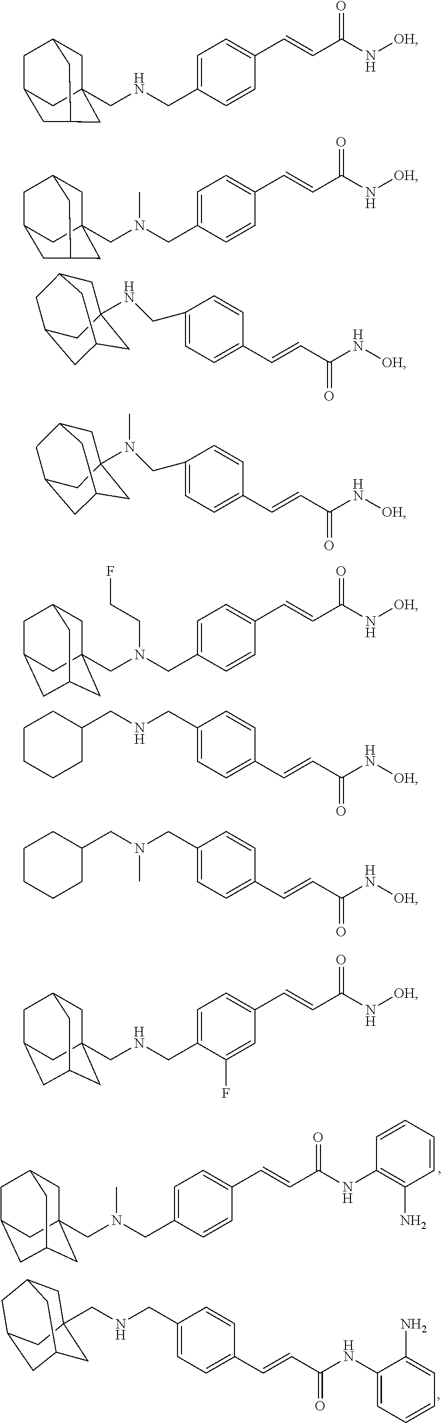

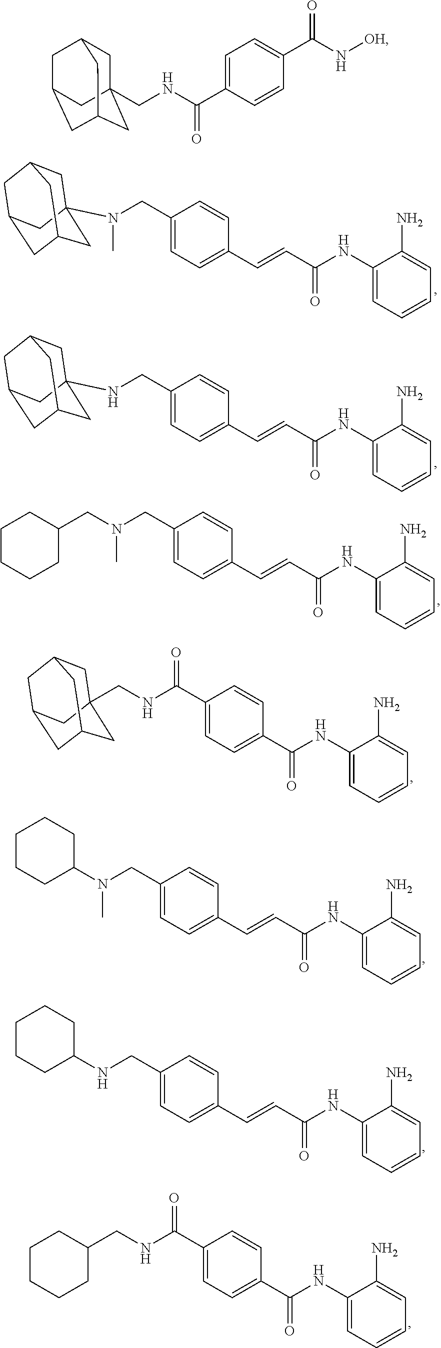

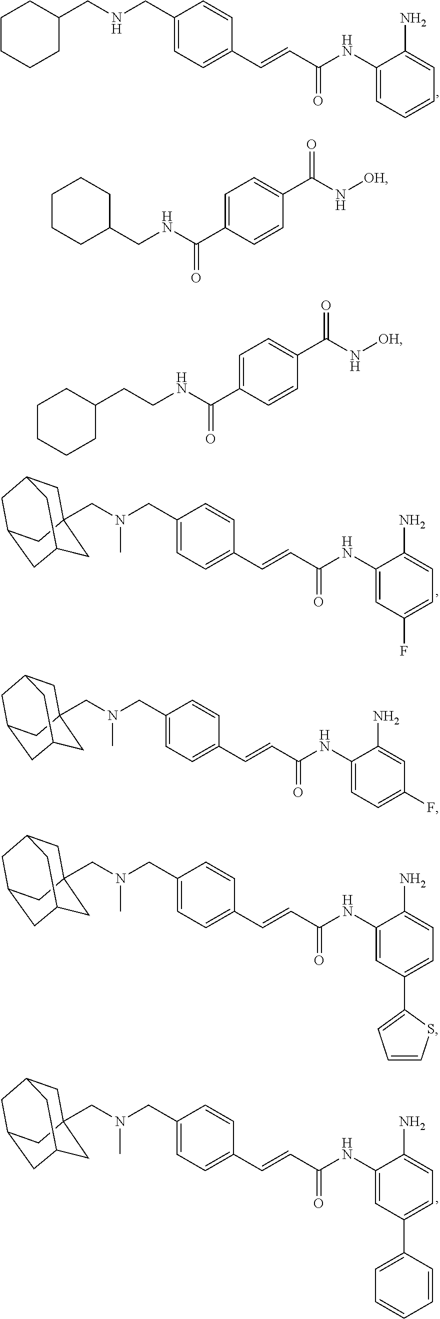

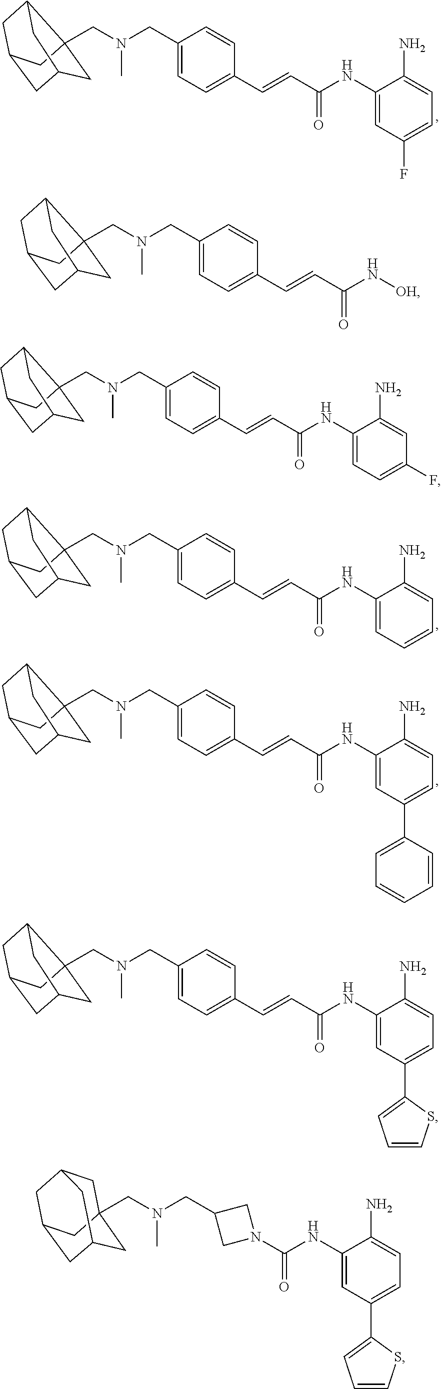

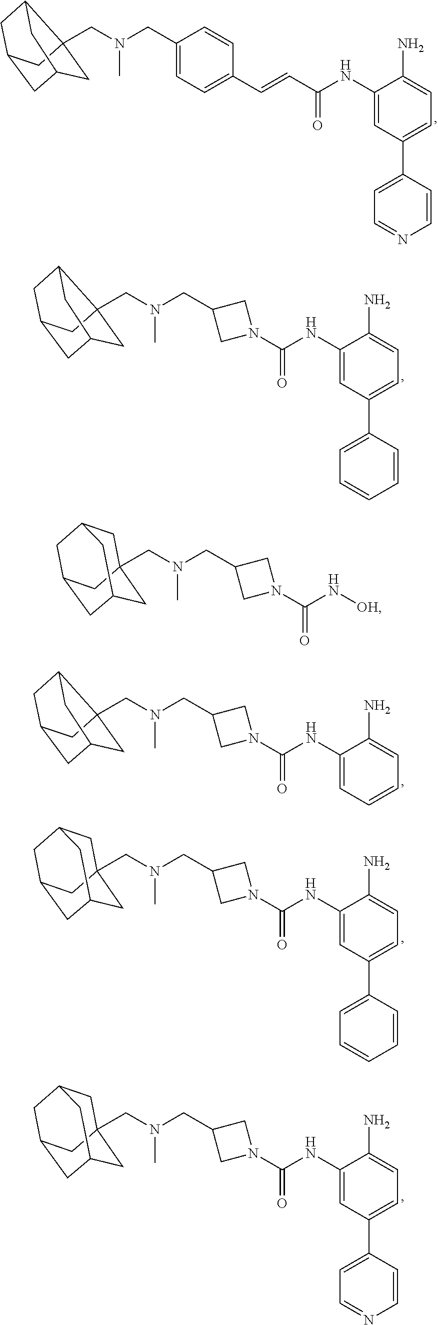 [SMILES: CN(Cc1ccc(/C=C/C(=O)Nc2cc(-c3ccncc3)ccc2N)cc1)CC12CC3CC(CC1C3)C2 ; CN(CC1CN(C(=O)Nc2cc(-c3ccccc3)ccc2N)C1)CC12CC3CC(CC1C3)C2 ; CN(CC1CN(C(=O)NO)C1)CC12CC3CC(CC(C3)C1)C2 ; CN(CC1CN(C(=O)Nc2ccccc2N)C1)CC12CC3CC(CC1C3)C2 ; CN(CC1CN(C(=O)Nc2cc(-c3ccccc3)ccc2N)C1)CC12CC3CC(CC(C3)C1)C2 ; CN(CC1CN(C(=O)Nc2cc(-c3ccncc3)ccc2N)C1)CC12CC3CC(CC(C3)C1)C2]

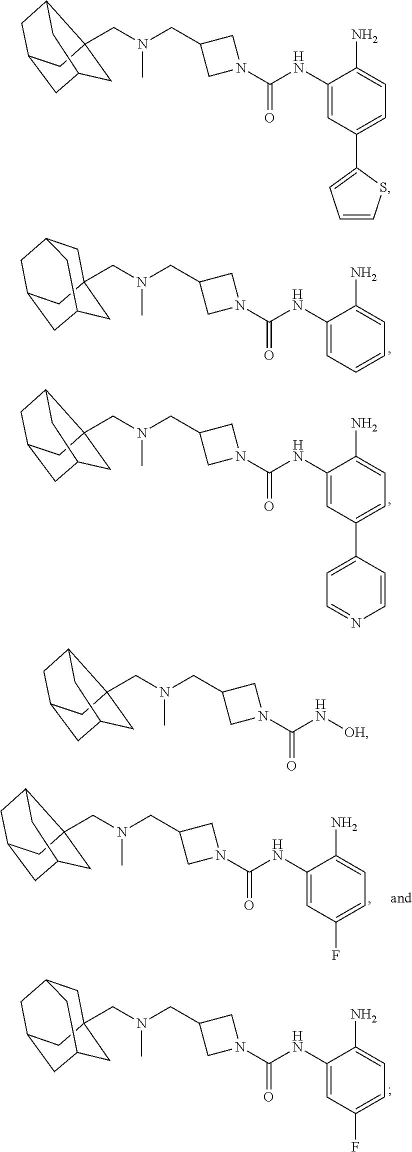 [SMILES: CN(CC1CN(C(=O)Nc2cc(-c3cccs3)ccc2N)C1)CC12CC3CC(CC1C3)C2 ; CN(CC1CN(C(=O)Nc2ccccc2N)C1)CC12CC3CC(CC(C3)C1)C2 ; CN(CC1CN(C(=O)Nc2cc(-c3ccncc3)ccc2N)C1)CC12CC3CC(CC1C3)C2 ; CN(CC1CN(C(=O)NO)C1)CC12CC3CC(CC1C3)C2 ; CN(CC1CN(C(=O)Nc2cc(F)ccc2N)C1)CC12CC3CC(CC1C3)C2 ; CN(CC1CN(C(=O)Nc2cc(F)ccc2N)C1)CC12CC3CC(CC(C3)C1)C2]

or a pharmaceutically acceptable salt thereof.

In some embodiments, the compound of Formula (Ia) is selected from the group consisting of:

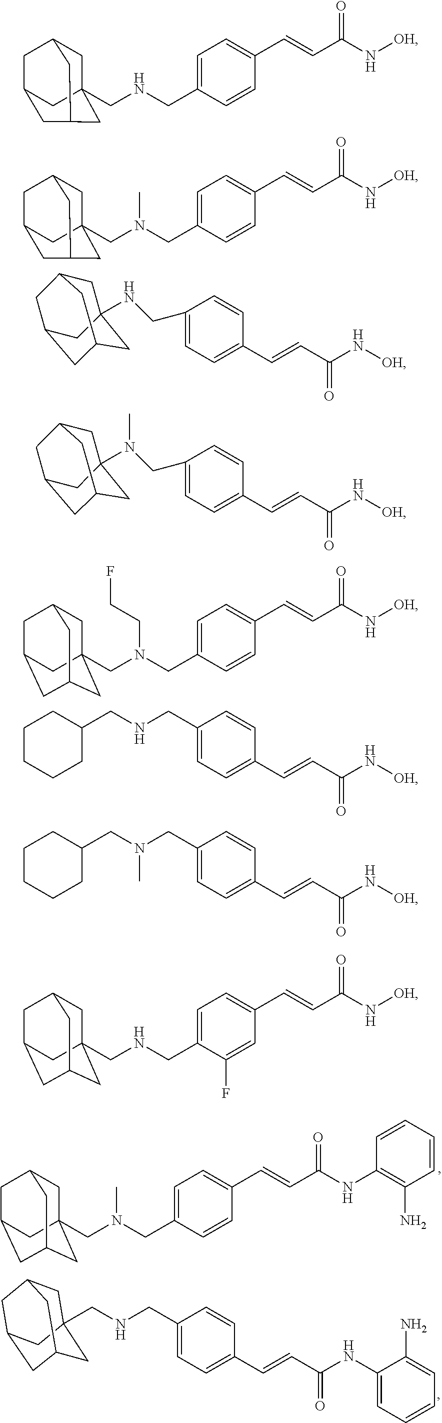

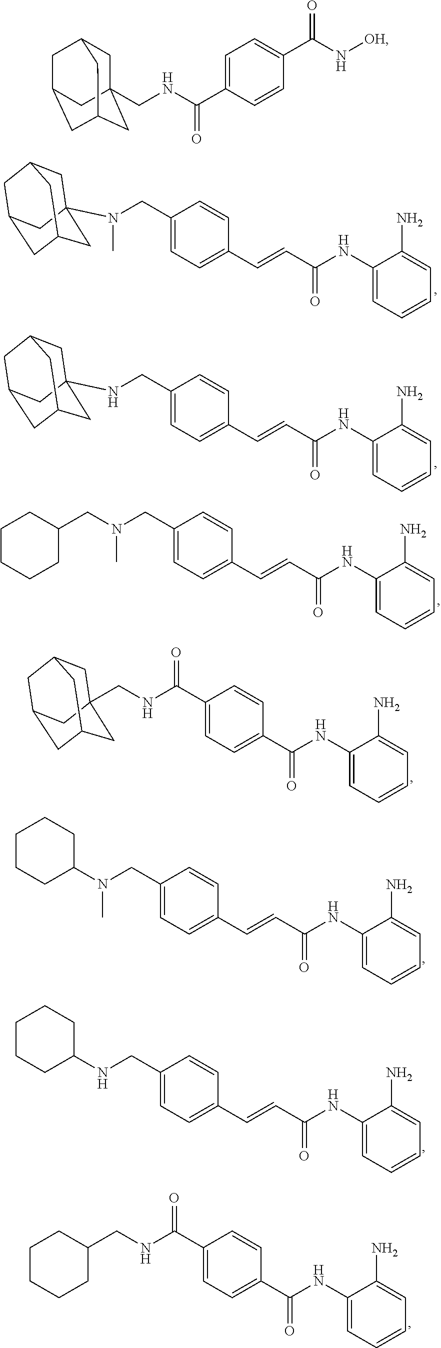

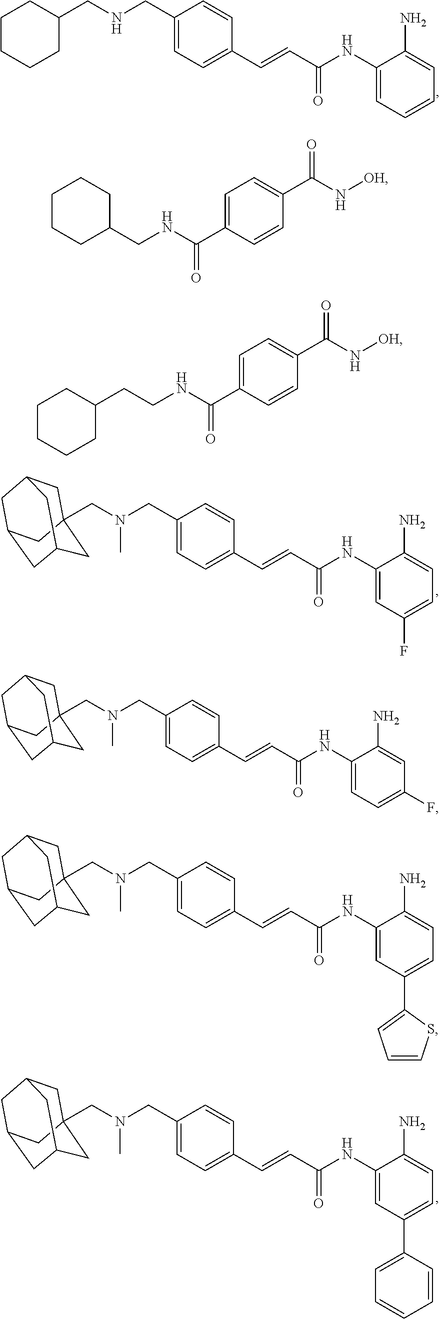

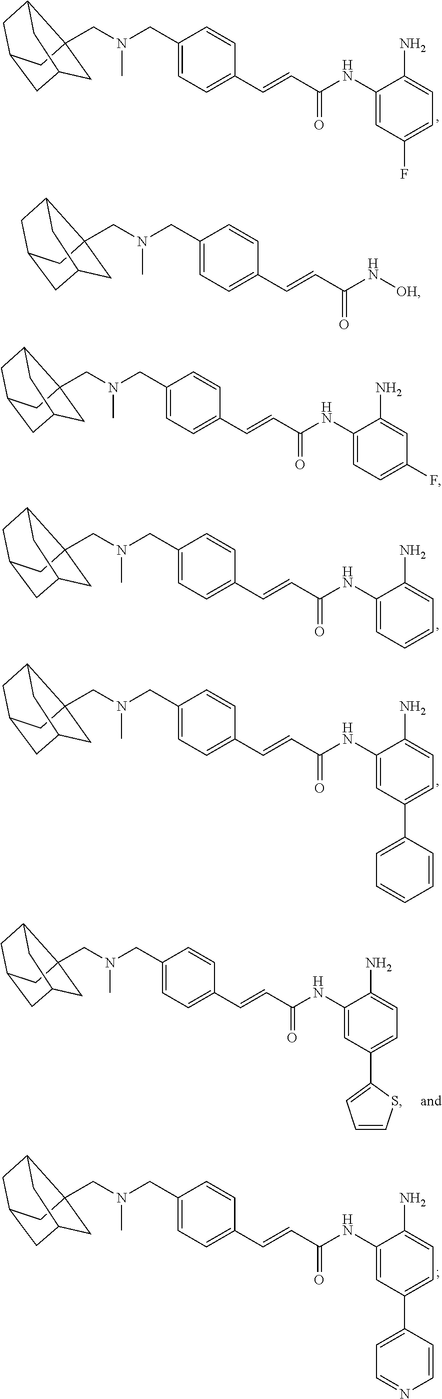

or a pharmaceutically acceptable salt thereof.

In some embodiments, the compound of Formula (I) and the compound of Formula (Ia) do not include a compound selected from the group consisting of:

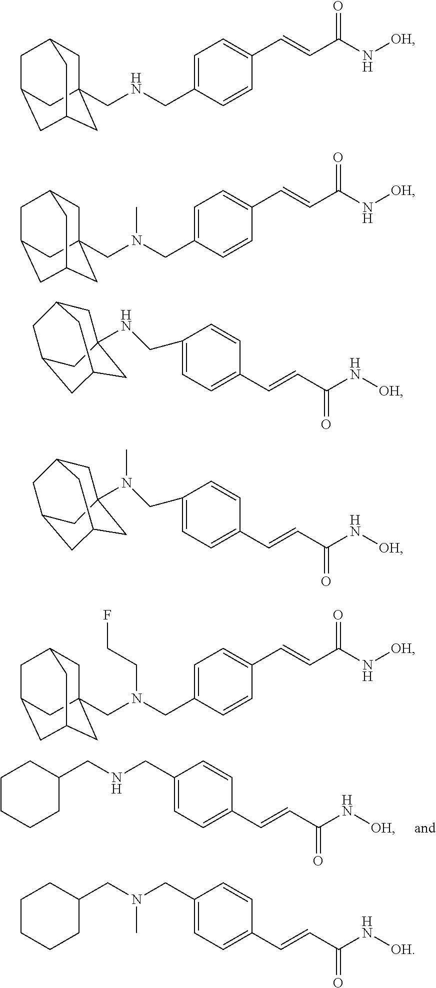

In some embodiments, the compound of Formula (I) or the compound of Formula (Ia) is:

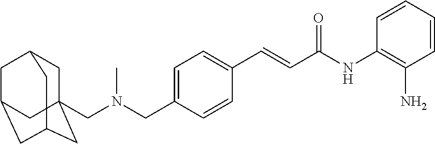 [SMILES: CN(Cc1ccc(/C=C/C(=O)Nc2ccccc2N)cc1)CC12CC3CC(CC(C3)C1)C2]

or a pharmaceutically acceptable salt thereof.

### Synthesis

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include methoxy, ethoxy, propoxy (e.g., n-propoxy and isopropoxy), tert-butoxy, and the like. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to about 20 carbon atoms, from 6 to about 15 carbon atoms, or from 6 to about 10 carbon atoms. In some embodiments, the aryl group is a substituted or unsubstituted phenyl.

As used herein, the term “carbamyl” to a group of formula —C(O)NH₂.

As used herein, the term “carbonyl”, employed alone or in combination with other terms, refers to a —C(═O)— group, which may also be written as C(O).

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, the term “C₁₋₃ alkoxy-C₁₋₃ alkyl” refers to a group of formula —(C₁₋₃ alkylene)-O(C₁₋₃ alkyl).

As used herein, the term “C₁₋₄ alkoxy-C₁₋₄ alkyl” refers to a group of formula —(C₁₋₄ alkylene)-O(C₁₋₄ alkyl).

As used herein, the term “C₁₋₄ haloalkoxy-C₁₋₄ alkyl” refers to a group of formula —(C₁₋₄ alkylene)-O(C₁₋₄ haloalkyl).

As used herein, the term “carboxy” refers to a group of formula —C(O)OH.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, “halo” refers to F, Cl, Br, or I. In some embodiments, the halo group is F or Cl.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, 6, 7, 8, 9, or 10 ring-forming carbons (C₃₋₁₀). Ring-forming carbon atoms of a cycloalkyl group can be optionally substituted by oxo or sulfido (e.g., C(O) or C(S)). Cycloalkyl groups also include cycloalkylidenes. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, cyclohexyl, cyclopentyl, or adamantyl. In some embodiments, the cycloalkyl has 6-10 ring-forming carbon atoms. In some embodiments, cycloalkyl is cyclohexyl or adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds provided herein can also include all isotopes of atoms occurring in the intermediates or final compounds. Isotopes include those atoms having the same atomic number but different mass numbers. For example, isotopes of hydrogen include tritium and deuterium.

All compounds, and pharmaceutically acceptable salts thereof, can be found together with other substances such as water and solvents (e.g. hydrates and solvates) or can be isolated.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977).

The compounds of Formula (I) provided herein can be prepared using methods analogous to those shown below in Scheme I, by substituting the appropriate starting materials. As will be appreciated, the compounds provided herein, including salts thereof, can be prepared using known organic synthesis techniques and can be synthesized according to any of numerous possible synthetic routes. For example, the condensation of amine (i) with substituted benzaldehyde (ii), and subsequent amine reduction affords secondary amine (iii). An optional step includes alkylation of the secondary amine to form a tertiary amine group (iv), and saponification of the ester moiety yields the corresponding carboxylic acid (v), which is coupled to aryl amine (vi) in the presence of N-(3-dimethylaminopropyl)-N′-ethylcarbodiimide hydrochloride (EDC.HCl) to afford the BOC-protected aryl amine (vii). Amine deprotection using standard procedures (e.g., reaction with a strong acid) yields the compound of Formula (I) (e.g., the compound of Example 2, shown below).

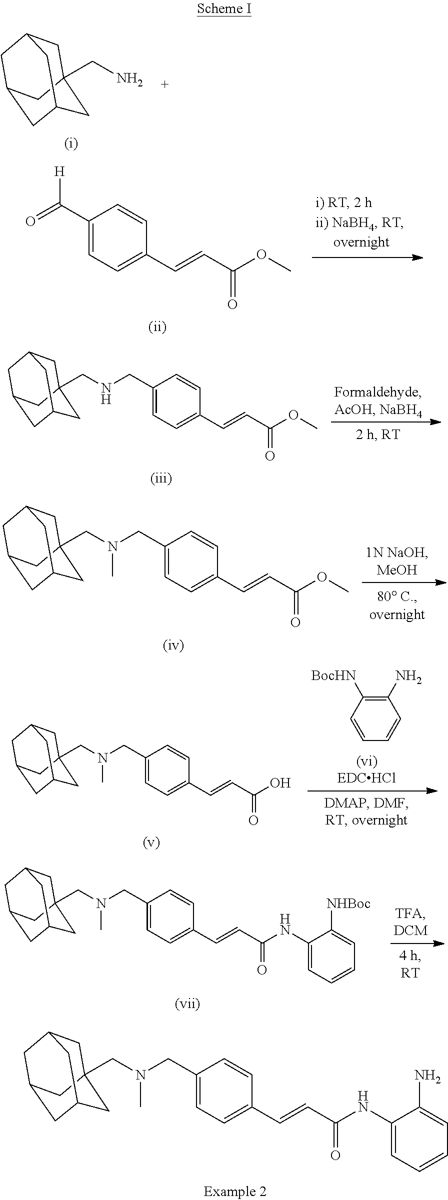

### Methods

Provided herein are methods of inhibiting of histone deacetylase (HDAC) in a patient. As used herein, the term “patient,” refers to any animal, including mammals. For example, mice, rats, other rodents, rabbits, dogs, cats, swine, cattle, sheep, horses, primates, and humans. In some embodiments, the patient is a human or rat. In some embodiments, the patient is a human. The present application further provides methods of inhibiting an activity of a histone deacetylase (HDAC) enzyme, comprising contacting the HDAC enzyme with an HDAC inhibitor provided herein, or a pharmaceutically acceptable salt thereof. In some embodiments, inhibiting an activity of a histone deacetylase (HDAC) enzyme comprises deregulating the histone deacetylase (HDAC) enzyme.

In some embodiments, about 0.1% to about 5% of the HDAC inhibitor administered to the patient crosses the blood brain barrier, for example, from about 0.1% to about 4%, from about 0.1% to about 3%, from about 0.1% to about 2%, from about 0.1% to about 1%, from about 0.1% to about 0.75%, from about 0.1% to about 0.5%, or from about 0.1% to about 0.25%. In some embodiments, about 0.5% of the compound administered crosses the blood brain barrier. In some embodiments, the histone deacetylase (HDAC) inhibitor administered to the patient has a brain:plasma ratio of at least about 1:1 to about 100:1, for example, a brain:plasma ratio of at least about 1:1, at least about 2:1, at least about 3:1, at least about 4:1, at least about 5:1, at least about 10:1, at least about 15:1, at least about 20:1, at least about 30:1, at least about 1:40, at least about 50:1, at least about 60:1, at least about 70:1, at least about 80:1, at least about 90:1, at least about 100:1, at least about 1:2, at least about 1:3, at least about 1:4, at least about 1:5, at least about 1:10, at least about 1:15, at least about 1:20, at least about 1:30, at least about 1:40, at least about 1:50, at least about 1:60, at least about 1:70, at least about 1:80, at least about 1:90, at least about 1:100, at least about 2:3, at least about 2:5, at least about 3:2, at least about 3:4, at least about 3:5, at least about 4:3, at least about 4:5. In some embodiments, the blood:plasma ratio is from about 1:1 to about 100:1, for example, from about 1:1 to about:1, from about 1:1 to about 3:1, from about 1:1 to about 4:1, from about 1:1 to about 5:1, from about 1:1 to about 10:1, from about 1:1 to about 15:1, from about 1:1 to about 20:1, from about 1:1 to about 30:1, from about 1:1 to about 1:40, from about 1:1 to about 50:1, from about 1:1 to about 60:1, from about 1:1 to about 70:1, from about 1:1 to about 80:1, from about 1:1 to about 90:1, from about 1:1 to about 100:1, from about 1:1 to about 3:2, or from about 1:1 to about 4:3. In some embodiments, the blood:plasma ratio is from about 1:100 to about 1:1, for example, from about 1:100 to about 1:1, from about 1:100 to about 1:2, from about 1:100 to about 1:3, from about 1:100 to about 1:4, from about 1:100 to about 1:5, from about 1:100 to about 1:10, from about 1:100 to about 1:15, from about 1:100 to about 1:20, from about 1:100 to about 1:30, from about 1:100 to about 1:40, from about 1:100 to about 1:50, from about 1:100 to about 1:60, from about 1:100 to about 1:70, from about 1:100 to about 1:80, from about 1:100 to about 1:90, at least about 1:100, from about 1:100 to about 2:3, from about 1:100 to about 2:5, from about 1:100 to about 3:4, from about 1:100 to about 3:5, or from about 1:100 to about 4:5.

The compounds provided herein can be selective HDAC inhibitors. As used, the term “selective” means that the compound binds to or inhibits a particular enzyme with greater affinity or potency, respectively, as compared to at least one other enzyme. In some embodiments, selectivity can be at least about, 10-fold, at least about 20-fold, at least about 50-fold, at least about 100-fold, at least about 200-fold, at least about 500-fold or at least about 1000-fold. Selectivity can be measured by methods routine in the art including that HDAC 1/2/3 selectivity are measured using the method described in Example 4. In some embodiments, selectivity can be tested at the Kₘ ATP concentration of each enzyme. In some embodiments, the compounds provided are selective Class I HDAC inhibitors. In some embodiments, the compounds provided are selective inhibitors of HDAC1 and HDAC2 over HDAC3. In some embodiments, the compounds provided are selective inhibitors of HDAC3 over HDAC1 and HDAC2.

Also provided are methods of treating a disease in a patient in need thereof, wherein the disease is associated with histone deacetylase. In some embodiments, the method comprises administering to the patient a therapeutically effective amount of a histone deacetylase inhibitor provided herein (e.g., a compound of Formula (I)), or a pharmaceutically acceptable salt thereof, wherein the disease is selected from the group consisting of cancer, a disease of the central nervous system, and an inflammatory autoimmune disease. In some embodiments, the disease is cancer. In some embodiments, the disease is a disease of the central nervous system. In some embodiments, the disease is an inflammatory autoimmune disease.

In some embodiments, the cancer is selected from the group consisting of breast cancer, prostate cancer, colon cancer, endometrial cancer, brain cancer, bladder cancer, skin cancer, cancer of the uterus, cancer of the ovary, lung cancer, pancreatic cancer, renal cancer, gastric cancer, and hematological cancer. In some embodiments, the cancer comprises a solid tumor. In some embodiments, the cancer is selected from the group consisting of glioma, glioblastoma, non-small cell lung cancer, and hematological cancer. In some embodiments, the cancer is associated with abnormal expression or abnormal activity of a histone deacetylase (HDAC) enzyme. In some embodiments, the cancer is associated with abnormal expression of a histone deacetylase (HDAC). In some embodiments, the cancer is associated with abnormal activity of a histone deacetylase (HDAC).

In some embodiments, a hematological cancer is selected from the group consisting of acute myeloblastic leukemia, chronic myeloid leukemia, B cell lymphoma, chronic lymphocytic leukemia (CLL), Non-Hodgkins lymphoma, hairy cell leukemia, Mantle cell lymphoma, Burkitt lymphoma, small lymphocytic lymphoma, follicular lymphoma, lymphoplasmacytic lymphoma, extranodal marginal zone lymphoma, activated B-cell like (ABC) diffuse large B cell lymphoma, and germinal center B cell (GCB) diffuse large B cell lymphoma. In some embodiments, the hematological cancer is selected from leukemia or lymphoma.

In some embodiments, a disease of the central nervous system is selected from the group consisting of Alzehimer's disease, attention deficit/hyperactivity disorder (ADHD), Bell's Palsy, bipolar disorder, catalepsy, Cerebal Palsy, epilepsy, encephalitis, Huntington's disease, locked-in syndrome, meningitis, migraine, multiple sclerosis (MS), Parkinson's disease, schizophrenia, tropical spastic paraparesis, and Tourette's syndrome. In some embodiments, the disease of the central nervous system is selected from the group consisting of Alzheimer's disease, bipolar disorder, depression, Huntington's disease, and schizophrenia. In some embodiments, the disease of the central nervous system further comprises depression. In some embodiments, the disease of the central nervous system comprises a neurodegenerative disease. In some embodiments, the disease of the central nervous system is associated with abnormal expression or abnormal activity of a histone deacetylase (HDAC) enzyme. In some embodiments, the disease of the central nervous system is associated with abnormal expression of a histone deacetylase (HDAC). In some embodiments, the disease of the central nervous system is associated with abnormal activity of a histone deacetylase (HDAC).

In some embodiments, an inflammatory autoimmune disease is selected from the group consisting of alopecia areata, autoimmune hemolytic anemia, autoimmune hepatitis, dermatomyositis, diabetes (type 1), juvenile idiopathic arthritis, glomerulonephritis, Graves' disease, Guillain-Barre syndrome, idiopathic thrombocytopenic purpura, myasthenia gravis, myocarditis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, thyroiditis, uveitis, vitiligo, and granulomatosis with polyangiitis (Wegener's granulomatosis). In some embodiments, the inflammatory autoimmune disease is associated with abnormal expression or abnormal activity of a histone deacetylase (HDAC) enzyme. In some embodiments, the inflammatory autoimmune disease is associated with abnormal expression of a histone deacetylase (HDAC). In some embodiments, the inflammatory autoimmune disease is associated with abnormal activity of a histone deacetylase (HDAC).

The present application further provides a method of treating a cancer in a patient, the method comprising:

i) identifying the cancer as being associated with abnormal activity or abnormal expression of a histone deacetylase (HDAC); and

ii) if the cancer is identified as being associated with abnormal activity of a histone deacetylase (HDAC), then administering to the patient a therapeutically effective amount of a compound of Formula (I), or a pharmaceutically acceptable salt thereof.

In some embodiments, the method comprises identifying a cancer as being associated with abnormal activity of a histone deacetylase (HDAC) and if the cancer is identified as being associated with abnormal activity of a histone deacetylase (HDAC), then administering to the patient a therapeutically effective amount of a compound of Formula (I), or a pharmaceutically acceptable salt thereof. In some embodiments, the method comprises identifying a cancer as being associated with abnormal expression of a histone deacetylase (HDAC) and if the cancer is identified as being associated with abnormal expression of a histone deacetylase (HDAC), then administering to the patient a therapeutically effective amount of a compound of Formula (I), or a pharmaceutically acceptable salt thereof. In some embodiments, the compound of Formula (I) is a compound of Formula (Ia), or a pharmaceutically acceptable salt thereof.

The present application further provides a method of treating a disease of the central nervous in a patient, the method comprising:

i) identifying the disease of the central nervous system as being associated with abnormal activity or abnormal expression of a histone deacetylase (HDAC); and

ii) if the disease of the central nervous system is identified as being associated with abnormal activity or abnormal expression of a histone deacetylase (HDAC), then administering to the patient a therapeutically effective amount of a compound of Formula (I), or a pharmaceutically acceptable salt thereof.

In some embodiments, the method comprises identifying a disease of the central nervous system as being associated with abnormal activity of a histone deacetylase (HDAC) and if the disease of the central nervous system is identified as being associated with abnormal activity of a histone deacetylase (HDAC), then administering to the patient a therapeutically effective amount of a compound of Formula (I), or a pharmaceutically acceptable salt thereof. In some embodiments, the method comprises identifying a disease of the central nervous system as being associated with abnormal expression of a histone deacetylase (HDAC) and if the disease of the central nervous system is identified as being associated with abnormal expression of a histone deacetylase (HDAC), then administering to the patient a therapeutically effective amount of a compound of Formula (I), or a pharmaceutically acceptable salt thereof. In some embodiments, the compound of Formula (I), is a compound of Formula (Ia), or a pharmaceutically acceptable salt thereof.

The present application further provides a method of treating an inflammatory autoimmune disease in a patient, the method comprising:

i) identifying the inflammatory autoimmune disease as being associated with abnormal activity or abnormal expression of a histone deacetylase (HDAC); and

ii) if the inflammatory autoimmune disease is identified as being associated with abnormal activity or abnormal expression of a histone deacetylase (HDAC), then administering to the patient a therapeutically effective amount of a compound of Formula (I), or a pharmaceutically acceptable salt thereof.

In some embodiments, the method comprises identifying an inflammatory autoimmune disease as being associated with abnormal activity of a histone deacetylase (HDAC) and if the inflammatory autoimmune disease is identified as being associated with abnormal activity of a histone deacetylase (HDAC), then administering to the patient a therapeutically effective amount of a compound of Formula (I), or a pharmaceutically acceptable salt thereof. In some embodiments, the method comprises identifying an inflammatory autoimmune disease as being associated with abnormal expression of a histone deacetylase (HDAC) and if the inflammatory autoimmune disease is identified as being associated with abnormal expression of a histone deacetylase (HDAC), then administering to the patient a therapeutically effective amount of a compound of Formula (I), or a pharmaceutically acceptable salt thereof. In some embodiments, the compound of Formula (I) is a compound of Formula (Ia), or a pharmaceutically acceptable salt thereof.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician. In some embodiments, the dosage of the compound, or a pharmaceutically acceptable salt thereof, administered to a patient or individual is about 1 mg to about 2 g, about 1 mg to about 1000 mg, about 1 mg to about 500 mg, about 1 mg to about 100 mg, about 1 mg to 50 mg, or about 50 mg to about 500 mg.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

### Combination Therapies

One or more additional pharmaceutical agents such as, for example, chemotherapeutics, anti-inflammatory agents, steroids, immunosuppressants, or other agents such as therapeutic antibodies, can be used in combination with the compounds of the present application for treatment of HDAC associated diseases, disorders or conditions. The one or more additional pharmaceutical agents can be administered to a patient simultaneously or sequentially.

Example antibodies for use in combination therapy include but are not limited to trastuzumab (e.g. anti-HER2), ranibizumab (e.g. anti-VEGF-A), bevacizumab (e.g. anti-VEGF), panitumumab (e.g. anti-EGFR), cetuximab (e.g. anti-EGFR), rituxan (anti-CD20) and antibodies directed to c-MET.

Example chemotherapeutics include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as cortisone, dexamethasone, hydrocortisone, methylprednisolone, prednisolone, prednisone, and the like.

Example anti-inflammatory compounds include aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, valdecoxib, and the like.

Example immunosuppressants include azathioprine, chlorambucil, cyclophosphamide, cyclosporine, daclizumab, infliximab, methotrexate, tacrolimus, and the like.

One or more of the following agents may be used in combination with the compounds provided herein and are presented as a non-limiting list: a cytostatic agent, cisplatin, doxorubicin, taxol, etoposide, irinotecan, topotecan, paclitaxel, docetaxel, epothilones, tamoxifen, 5-fluorouracil, methotrexate, temozolomide, cyclophosphamide, SCH 66336, R115777, L778,123, BMS 214662, gefitinib, erlotinib hydrochloride, antibodies to EGFR, imatinib mesylate, intron, ara-C, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, folinic acid, pentostatin, vinblastine, vincristine, vindesine, bleomycin, dactinomycin, daunorubicin, epirubicin, idarubicin, mithramycin, deoxycoformycin, mitomycin-C, L-asparaginase, teniposide, 17α-ethinylestradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, vinorelbine, anastrazole, letrozole, capecitabine, reloxafine, hexamethylmelamine, bevacizumab, bexxar, velcade, zevalin, trisenox, xeloda, porfimer, erbitux, thiotepa, altretamine, trastuzumab, fulvestrant, exemestane, rituximab, C225, alemtuzumab, clofarabine, cladribine, aphidicolin, sunitinib, dasatinib, tezacitabine, Smll, triapine, didox, trimidox, amidox, 3-AP, MDL-101,731, bendamustine, ofatumumab, and GS-1101 (also known as CAL-101).

### Pharmaceutical Formulations and Dosage Forms

When employed as pharmaceuticals, the compounds provided herein can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, (e.g., intrathecal or intraventricular, administration). Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided are pharmaceutical compositions which contain, as the active ingredient, a histone deacetylase (HDAC) inhibitor provided herein (e.g., a compound of Formula (I)), or a pharmaceutically acceptable salt thereof, in combination with one or more pharmaceutically acceptable carriers (e.g., an excipient). In some embodiments, the composition is suitable for intraperitoneal administration. In some embodiments, the composition is suitable for intravenous administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation.

The compounds provided herein may be milled using known milling procedures such as wet milling to obtain a particle size appropriate for tablet formation and for other formulation types. Finely divided (nanoparticulate) preparations of the compounds provided herein can be prepared by processes known in the art, (e.g., see International App. No. WO 2002/000196).

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions provided herein can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions provided herein contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions provided herein contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions provided herein contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses also provided.

The active compound can be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present application. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present application.

The tablets or pills of the present application can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present application can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between about 3 and about 11. In some embodiments, the pH is from about 5 to about 9. In some embodiments, the pH is from about 7 to about 8. It will be understood that use of certain of the foregoing excipients, carriers, or stabilizers will result in the formation of pharmaceutical salts.

The therapeutic dosage of a compound of the present application can vary according to, for example, the particular use for which the treatment is made, the manner of administration of the compound, the health and condition of the patient, and the judgment of the prescribing physician. The proportion or concentration of a compound provided herein in a pharmaceutical composition can vary depending upon a number of factors including dosage, chemical characteristics (e.g., hydrophobicity), and the route of administration. For example, the compounds provided can be administered as an aqueous physiological buffer solution containing about 0.1 to about 10% w/v of the compound for parenteral administration. Some typical dose ranges are from about 1 jg/kg to about 1 g/kg of body weight per day. In some embodiments, the dose range is from about 0.01 mg/kg to about 100 mg/kg of body weight per day, for example, from about 0.01 mg/kg to about 50 mg/kg, about 0.01 mg/kg to about 25 mg/kg, about 0.01 mg/kg to about 10 mg/kg, about 0.01 mg/kg to about 5 mg/kg, about 0.01 mg/kg to about 2 mg/kg, or about 0.01 mg/kg to about 1 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions provided can further include one or more additional pharmaceutical agents such as a chemotherapeutic, a steroid, an anti-inflammatory compound, or an immunosuppressant, examples of which are listed herein.

### Labeled Compounds and Assay Methods

Another aspect of the present application relates to labeled histone deacetylase (HDAC) inhibitors (radio-labeled, fluorescent-labeled, etc.) that would be useful not only in imaging techniques but also in assays, both in vitro and in vivo, for localizing and quantitating HDAC in tissue samples, including human tissue samples, and for identifying HDAC ligands by inhibition binding of a labeled compound.

The present application further includes isotopically-labeled HDAC inhibitors provided herein (e.g., compounds of Formula (I)). An “isotopically” or “radio-labeled” compound is a compound provided herein where one or more atoms are replaced or substituted by an atom having an atomic mass or mass number different from the atomic mass or mass number typically found in nature (i.e., naturally occurring).

As used herein, the term “Ci”, used alone or in combination with other terms, refers to “Curie”, which one of ordinary skill will recognize as a unit of radioactivity that is standard in the art.

As used herein, the term “specific activity”, used alone or in combination with other terms, refers to the activity of a given radioisotope per unit mass, for example, Ci/g.

Suitable radionuclides that may be incorporated in compounds of the present application include but are not limited to ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro labeling and competition assays, compounds that incorporate ³H, ¹¹C, ¹⁴C, ⁸²Br, ¹²⁵, ¹³¹I, or ³⁵S will generally be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be useful.

It is understood that a “radio-labeled” or “labeled compound” is a compound that has incorporated at least one radionuclide. In some embodiments the radionuclide is selected from the group consisting of ³H, ¹¹C, ¹⁴C, ¹²⁵I, ³⁵S and ⁸²Br. In some embodiments, one or more H atoms for any compound described herein is each replaced by a deuterium atom. In some embodiments, one or more C atoms for any compound described herein is each replaced by a ¹¹C atom.

The present application can further include synthetic methods for incorporating radio-isotopes into compounds provided. Synthetic methods for incorporating radio-isotopes into organic compounds are well known in the art, and one of ordinary skill in the art will readily recognize the methods applicable for the compounds provided.

A labeled compound provided herein can be used in a screening assay to identify/evaluate compounds. For example, a newly synthesized or identified compound (i.e., test compound) which is labeled can be evaluated for its ability to bind an HDAC enzyme by monitoring its concentration variation when contacting with the HDAC enzyme, through tracking of the labeling. For example, a test compound (labeled) can be evaluated for its ability to reduce binding of another compound which is known to bind to an HDAC enzyme (i.e., standard compound). Accordingly, the ability of a test compound to compete with the standard compound for binding to the HDAC enzyme directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### EXAMPLES

### General Methods

All reagents and solvents were of ACS-grade purity or higher and used without further purification. NMR data were recorded on a Varian 500 MHz magnet and are reported in ppm units downfield from trimethylsilane.

### Example 1. (E)-3-(4-((((1r,3R,5S)-adamantan-1-ylmethyl)(methyl)amino)methyl) phenyl)-N-hydroxyacrylamide

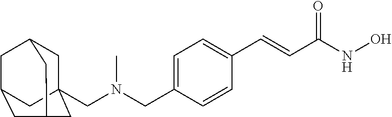 [SMILES: CN(Cc1ccc(/C=C/C(=O)NO)cc1)CC12CC3CC(CC(C3)C1)C2]

### Step 1. (E)-methyl 3-(4-((((3r,5r,7r)-adamantan-ylmethyl)amino)methyl)phenyl)acrylate

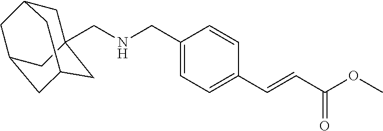 [SMILES: COC(=O)/C=C/c1ccc(CNCC23CC4CC(CC(C4)C2)C3)cc1]

Adamantan-1-ylmethanamine (1 g, 6.0 mmol) and (E)-methyl 3-(4-formylphenyl)acrylate (1 g, 5.3 mmol) was dissolved in MeOH (30 mL) and the mixture was stirred at room temperature for 2 h. Sodium borohydride (0.61 g, 16 mmol) was then added, and the suspension was stirred overnight at room temperature. The white precipitate was filtered and dried to obtain (E)-methyl 3-(4-((((3r,5r,7r)-adamantan-ylmethyl)amino)methyl)phenyl)acrylate (1.35 g, yield: 75%). ¹H-NMR (500 MHz, CDCl₃): δ 7.69 (d, J=16 Hz, 1H), 7.48 (d, J=7 Hz, 2H), 7.35 (d, J=7 Hz, 2H), 6.43 (d, J=16 Hz, 1H), 3.81 (s, 2H), 3.80 (s, 3H), 2.23 (s, 3H), 1.96 (s, 3H), 1.63-1.73 (m, 6H), 1.53 (s, 6H); ¹³C-NMR (125 MHz, CDCl₃): 167.55, 144.78, 143.81, 132.87, 128.35 (2C), 128.08 (2C), 117.10, 62.15, 54.28, 51.65, 40.85 (3C), 37.24 (3C), 33.49, 28.48 (3C). LC-MS calculated for C₂₂H₂₉NO₂ expected [M]: 339.2; Found [M+H]⁺: 340.3.

### Step 2. (E)-methyl 3-(4-((((3r,5r,7r)-adamantan-1-ylmethyl)(methyl)amino)methyl) phenyl)acrylate

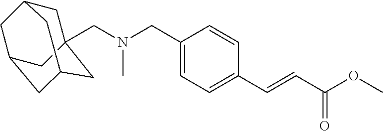 [SMILES: COC(=O)/C=C/c1ccc(CN(C)CC23CC4CC(CC(C4)C2)C3)cc1]

To a solution of (E)-methyl 3-(4-((((3r,5r,7r)-adamantan-ylmethyl)amino)methyl)phenyl)acrylate (0.5 g, 1.5 mmol) in MeOH (30 mL) was added formaldehyde (33% aq. solution, 2 mL) followed by acetic acid (0.1 mL). The mixture was stirred at room temperature for 2 h. Sodium borohydride (0.61 g, 16 mmol) was then added, and the suspension was stirred overnight at room temperature. The white precipitate was filtered and purified by flash chromatography in hexanes:ethyl acetate (4:1) to obtain (E)-methyl 3-(4-((((3r,5r,7r)-adamantan-1-ylmethyl)(methyl)amino) methyl)phenyl)acrylate (0.29 g, yield: 55%) as white solid. ¹H-NMR (500 MHz, MeOH-d₄): δ 7.55 (d, J=16 Hz, 1H), 7.52 (d, J=7.5 Hz, 2H), 7.37 (d, J=7.5 Hz, 2H), 6.47 (d, J=16 Hz, 1H), 3.77 (s, 2H), 2.21 (s, 2H), 1.94 (s, 3H), 1.66-1.76 (m, 6H), 1.54-1.55 (m, 6H); ¹³C-NMR (125 MHz, MeOH-d₄): 164.93, 141.32, 139.71, 133.76, 128.62 (2C), 127.43 (2C), 116.8901, 61.08, 53.32, 40.42 (3C), 36.78 (3C), 32.84, 28.48 (3C). LC-MS calculated for C₂₁H₂₈N₂O₂ expected [M]: 340.2; Found [M+H]⁺: 341.3.

### Step 3. (E)-3-(4-((((3r,5r,7r)-adamantan-1-ylmethyl)(methyl)amino)methyl)phenyl)-N hydroxyacrylamide

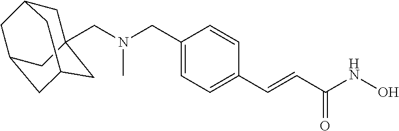 [SMILES: CN(Cc1ccc(/C=C/C(=O)NO)cc1)CC12CC3CC(CC(C3)C1)C2]

To a solution of (E)-methyl 3-(4-((((3r,5r,7r)-adamantan-1-ylmethyl)(methyl)amino)methyl)phenyl)acrylate (0.5 g, 1.4 mmol) in MeOH/THF (3 mL/3 mL) at 0° C. was added NH₂OH (50% aq. solution, 3 mL) followed by 1 M NaOH (2 mL). The mixture was stirred at 0° C. for 2 h, warmed to room temperature, and stirred for 2 h. Acidification with 1 M HCl to pH 7-8 resulted in product precipitation. The precipitate was filtered and dried to obtain the title compound (0.21 g, 42%) as white solid. ¹H-NMR (500 MHz, DMSO-d₆): δ 7.49 (d, J=7.5 Hz, 2H), 7.42 (d, J=16 Hz, 1H), 7.33 (d, J=7.5 Hz, 2H), 6.45 (d, J=16 Hz, 1H), 3.48 (s, 2H), 2.11 (s, 3H), 2.06 (s, 2H), 1.89 (s, 3H), 1.55-1.65 (m, 6H), 1.46 (s, 6H); δ ¹³C-NMR (125 MHz, DMSO-d₆): 163.15, 142.00, 138.43, 139.91, 129.36 (2C), 127.77 (2C), 119.06, 70.12, 64.57, 45.71, 41.01 (3C), 37.21 (3C), 35.27, 28.33 (3C). LC-MS calculated for C₂₂H₃₀N₂O₂ expected [M]: 354.2; Found [M+H]⁺: 355.3.

### Example 2. (E)-3-(4-((((3r,5r,7r)-adamantan-1-ylmethyl)(methyl)amino)methyl) phenyl)-N-(2-aminophenyl)acrylamide

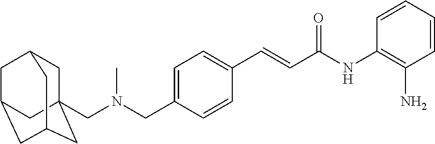 [SMILES: CN(Cc1ccc(/C=C/C(=O)Nc2ccccc2N)cc1)CC12CC3CC(CC(C3)C1)C2]

### Step 1. (E)-methyl 3-(4-((((3r,5r,7r)-adamantan-1-ylmethyl)amino)methyl)phenyl) acrylate

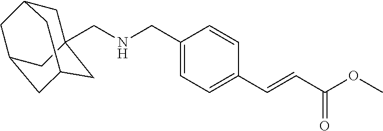 [SMILES: COC(=O)/C=C/c1ccc(CNCC23CC4CC(CC(C4)C2)C3)cc1]

Condensation of (3r,5r,7r)-adamantan-1-ylmethanamine with (E)-methyl 3-(4-formylphenyl)acrylate, and subsequent amine reduction in the presence of sodium borohydride afforded (E)-methyl 3-(4-((((3r,5r,7r)-adamantan-1-ylmethyl)amino)methyl)phenyl)acrylate.

### Step 2. (E)-methyl 3-(4-((((3r,5r,7r)-adamantan-1-ylmethyl)(methyl)amino)methyl) phenyl)acrylate

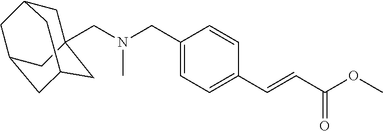 [SMILES: COC(=O)/C=C/c1ccc(CN(C)CC23CC4CC(CC(C4)C2)C3)cc1]

Alkylation of (E)-methyl 3-(4-((((3r,5r,7r)-adamantan-1-ylmethyl)amino)methyl)phenyl)acrylate in the presence of formaldehyde, acetic acid, and sodium borohydride afforded (E)-methyl 3-(4-((((3r,5r,7r)-adamantan-1-ylmethyl)(methyl)amino)methyl)phenyl)acrylate.

### Step 3. (E)-3-(4-((((3r,5r,7r)-adamantan-1-ylmethyl)(methyl)amino)methyl)phenyl) acrylic acid

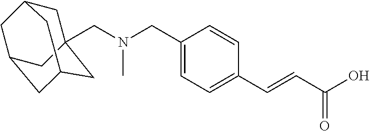 [SMILES: CN(Cc1ccc(/C=C/C(=O)O)cc1)CC12CC3CC(CC(C3)C1)C2]

Reaction of (E)-methyl 3-(4-((((3r,5r,7r)-adamantan-1-ylmethyl)(methyl)amino)methyl)phenyl)acrylate with 1 M NaOH in MeOH, heated to 80° C. overnight, and subsequent acidification afforded (E)-3-(4-((((3r,5r,7r)-adamantan-1-ylmethyl)(methyl)amino)methyl)phenyl)acrylic acid.

### Step. 4. tert-butyl (2-((E)-3-(4-((((3r,5r,7r)-adamantan-1-ylmethyl)(methyl)amino)methyl)phenyl)acrylamido)phenyl)carbamate

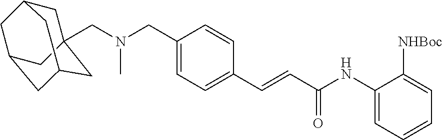 [SMILES: CN(Cc1ccc(/C=C/C(=O)Nc2ccccc2NC(=O)OC(C)(C)C)cc1)CC12CC3CC(CC(C3)C1)C2]

(E)-3-(4-((((3r,5r,7r)-adamantan-1-ylmethyl)(methyl)amino)methyl)phenyl) acrylic acid was reacted with tert-butyl (2-aminophenyl)carbamate in the presence of N-(3-dimethylaminopropyl)-N′-ethylcarbodiimide hydrochloride (EDC.HCl) to afford tert-butyl (2-((E)-3-(4-((((3r,5r,7r)-adamantan-1-ylmethyl)(methyl)amino)methyl)phenyl) acrylamido)phenyl)carbamate.

### Step 5. (E)-3-(4-((((3r,5r,7r)-adamantan-1-ylmethyl)(methyl)amino)methyl)phenyl)-N-(2-aminophenyl)acrylamide

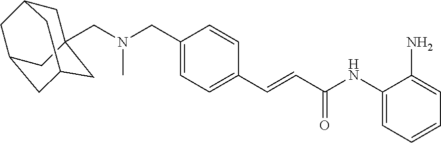 [SMILES: CN(Cc1ccc(/C=C/C(=O)Nc2ccccc2N)cc1)CC12CC3CC(CC(C3)C1)C2]

The title compound was prepared by reacting tert-butyl (2-((E)-3-(4-((((3r,5r,7r)-adamantan-1-ylmethyl)(methyl)amino)methyl)phenyl)acrylamido)phenyl)carbamate with trifluoroacetic acid in DCM over 4 h at room temperature.

### Example 3. ¹¹C Radiolabeled (E)-3-(4-((((1r,3R,5S)-adamantan-1-ylmethyl) (methyl)amino)methyl)phenyl)-N-hydroxyacrylamide

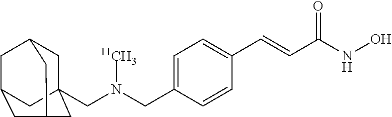 [SMILES: [11CH3]N(Cc1ccc(/C=C/C(=O)NO)cc1)CC12CC3CC(CC(C3)C1)C2]

[¹¹C]CH₃I was trapped in a TRACERlab FX-M synthesizer reactor (General Electric) preloaded with a solution of precursor (4) (1.0 mg) in dry DMSO (300 μL). The solution was stirred at 100° C. for 4 min and water (1.2 mL) was added. The reaction mixture was purified by reverse phase semi-preparative HPLC (Phenomenex Luna 5u C8(2), 250 mm×10 mm, 5 μm, 5.0 mL/min, 40% H₂O+ammonium acetate (0.1 M)/60% CH₃CN) and the desired fraction was collected. The final product was reformulated (purified and eluted in a solvent appropriate for in vivo injection) by loading onto a solid-phase exchange (SPE)C-18 cartridge, rinsing with IM NaOH aq. (5 mL), eluting with EtOH (1 mL), and diluting with 50 μL acetic acid in saline (0.9%, 9 mL). The chemical and radiochemical purity of the final product was tested by analytical HPLC (Agilent Eclipse XDB-C 18, 150 mm×4.6 mm). The identity of the product was confirmed by analytical HPLC with additional co-injection of unlabeled (E)-3-(4-((((1r,3R,5S)-adamantan-1-ylmethyl) (methyl)amino)methyl)phenyl)-N-hydroxyacrylamide reference standard. The average radiochemical yield was 3-5% (nondecay corrected to trapped [¹¹C]CH₃I). Chemical and radiochemical purities were >95% with a specific activity 1.0+0.2 Ci/mol at End of Bombardment (EOB).

### Example 4. HDAC Inhibition Assay

All recombinant human HDACs were purchased from BPS Bioscience. The substrates, Broad Substrate A and Broad Substrate B (see International App. No. WO 2013/067391), were synthesized in-house. All other reagents were purchased from Sigma-Aldrich. Caliper EZ reader II system was used to collect all data. HDAC inhibition assays: Compounds were tested in a 12-point dose curve with 3-fold serial dilution starting from 33.33 μM. Purified HDACs were incubated with 2 μM (the concentration is kept the same for all the HDACs, below the Michalis Constant, Km, of substrate) carboxyfluorescein (FAM)-labeled acetylated or trifluoroacetylated peptide substrate (Broad Substrate A and B respectively) and test compound for 60 min at room temperature, in HDAC assay buffer containing 50 mM HEPES (pH=7.4), 100 mM KCl, 0.01% BSA and 0.001% Tween-20. Reactions were terminated by the addition of 1.5 μM of the known pan-HDAC inhibitor LBH-589 (panobinostat). Substrate and product were separated electrophoretically, and fluorescence intensity of the substrate and product peaks was determined and analyzed by Labchip EZ Reader. The reactions were performed in duplicate for each sample. IC₅₀ values were automatically calculated by Origin 8 using 4 Parameter Logistic Model. The percent inhibition was plotted against the compound concentration, and the IC₅₀ value was determined from the logistic dose-response curve fitting by Origin 8.0 software. IC₅₀ values for example tested compounds are shown below in Tables 1A-1D.

TABLE 1A

| Compound | 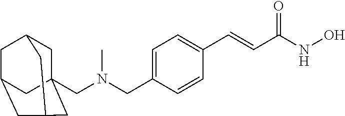 [SMILES: CN(Cc1ccc(/C=C/C(=O)NO)cc1)CC12CC3CC(CC(C3)C1)C2] |
|---|---|
| HDAC1 | 0.3 |
| IC₅₀ (nm) | |
| HDAC2 | 2.0 |
| IC₅₀ (nm) | |
| HDAC3 | 0.6 |
| IC₅₀ (nm) | |
| HDAC4 | 1970 |
| IC₅₀ (nm) | |
| HDAC5 | 352 |
| IC₅₀ (nm) | |
| HDAC6 | 4.1 |
| IC₅₀ (nm) | |
| HDAC7 | >20000 |
| IC₅₀ (nm) | |
| HDAC8 | >15000 |
| IC₅₀ (nm) | |
| HDAC9 | >15000 |
| IC₅₀ (nm) | |

TABLE 1B

| Compound | 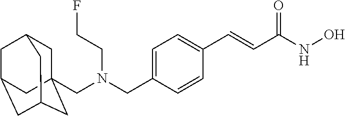 [SMILES: O=C(/C=C/c1ccc(CN(CCF)CC23CC4CC(CC(C4)C2)C3)cc1)NO] |
|---|---|
| HDAC1 | 222 |
| IC₅₀ (nm) | |
| HDAC2 | 651 |
| IC₅₀ (nm) | |
| HDAC3 | 470 |
| IC₅₀ (nm) | |
| HDAC4 | >12000 |
| IC₅₀ (nm) | |
| HDAC5 | >5000 |
| IC₅₀ (nm) | |
| HDAC6 | 308 |
| IC₅₀ (nm) | |
| HDAC7 | >30000 |
| IC₅₀ (nm) | |
| HDAC8 | >30000 |
| IC₅₀ (nm) | |
| HDAC9 | >30000 |
| IC₅₀ (nm) | |

TABLE 1C

| Compound | 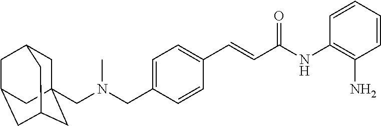 [SMILES: CN(Cc1ccc(/C=C/C(=O)Nc2ccccc2N)cc1)CC12CC3CC(CC(C3)C1)C2] |
|---|---|
| HDAC1 | 149 |
| IC₅₀ (nm) | |
| HDAC2 | 602 |
| IC₅₀ (nm) | |
| HDAC3 | 216 |
| IC₅₀ (nm) | |
| HDAC4 | — |
| IC₅₀ (nm) | |
| HDAC5 | >40000 |
| IC₅₀ (nm) | |
| HDAC6 | >10000 |
| IC₅₀ (nm) | |
| HDAC7 | >40000 |
| IC₅₀ (nm) | |
| HDAC8 | >40000 |
| IC₅₀ (nm) | |
| HDAC9 | — |
| IC₅₀ (nm) | |

TABLE 1D

| Compound | 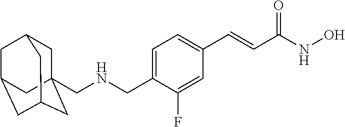 [SMILES: O=C(/C=C/c1ccc(CNCC23CC4CC(CC(C4)C2)C3)c(F)c1)NO] |
|---|---|
| HDAC1 | 0.8 |
| IC₅₀ (nm) | |
| HDAC2 | 6.4 |
| IC₅₀ (nm) | |
| HDAC3 | 0.5 |
| IC₅₀ (nm) | |
| HDAC4 | >70000 |
| IC₅₀ (nm) | |
| HDAC5 | 4308 |
| IC₅₀ (nm) | |
| HDAC6 | >40000 |
| IC₅₀ (nm) | |
| HDAC7 | >70000 |
| IC₅₀ (nm) | |
| HDAC8 | >5000 |
| IC₅₀ (nm) | |
| HDAC9 | >50000 |
| IC₅₀ (nm) | |

### Example 5. Mouse Primary Neuronal Histone Acetylation Assay

Mouse primary neuronal cultures were generated in factory precoated poly-D-lysine 96-well plates (BD Biosciences #BD356692) treated overnight with 75 μL/well of laminin [0.05 mg/mL] (Sigma #L2020) in PBS buffer. E15 embryonic mouse forebrain was dissociated into a single cell suspension by gentle trituration following trypsin/DNAse digestion (trypsin: Cellgro #25-052-Cl; DNAse: Sigma #D4527). 12,500 cells per well were plated in 100 μL Neurobasal medium (Gibco #21103-049) containing 2% B27 (Gibco #17504-044), 1% Penicillin/Streptomycin/Glutamine (Cellgro #30-009-CI) and cultured at 37° C. with 5% CO₂. After 13 days, cultures were treated with HDAC inhibitors by pin transfer of each compound (185 nL per well) using a CyBi-Well vario pinning robot (CyBio Corp., Germany) and subsequently incubated for 24 h at 37° C. with 5% CO₂, then fixed in 4% formaldehyde for 10 min. Following two washes with phosphate-buffered saline, cells were permeabilized and blocked with a solution of 0.1% Triton X-100 and 2% BSA in PBS. Cells were stained with an anti-Ac-H4K12 antibody (Millipore, catalog #04-119) and Alexa488-conjugated secondary antibody (Molecular Probes). Cell nuclei were identified by staining with Hoechst 33342 (Invitrogen, H3570). Cell nuclei and histone acetylation signal intensity were detected and measured using a laser-scanning microcytometer (Acumen eX3, TTP Laptech). Acumen Explorer software was used to identify a threshold of histone acetylation signal intensity such that, without HDAC inhibitor, >99.5% of cells had intensity levels below the threshold. In the presence of HDAC inhibitors, cells signal intensities above the threshold were scored as “bright green cells” and expressed as a percentage normalized to DMSO controls. EC₅₀ values were determined from curve fitting using GraphPad Prism v5 software (GraphPad Software, Inc. La Jolla, Calif., USA).

### Example 6. Ex Vivo Autoradiography

20 μm thick sagittal rat brain sections were cut using a −20° C. cryostat, thaw-mounted onto gelatin-coated slides, fixed in 4% paraformaldehyde containing 2% ethanol for 60 min at 4° C. and washed 10 min in ice-cold 10 mM Tris-HCl, pH 7.4. Sections were then incubated at room temperature in a 50 mL bath for 2 hr to allow for slow HDAC binding kinetics. All incubation baths contained 10 mM Tris-HCl+5% DMSO, pH 7.4. The radiolabeled compound of Example 3 was prepared as described and 100 microCuries (μCi) was added to each bath. Following 10 min incubation at room temperature, sections were washed 2×1 min in 10 mM Tris-HCl (pH 7.4) and carefully wicked dry on absorbent towels. Sections were exposed for 1 hour to a multisensitive phosphorscreen and developed using a Cyclone Plus phosphorimager (both from Perkin Elmer). The resulting parent image was evaluated using ImageJ software (NIH) with JET color lookup table (LUT) with whole-image intensity adjusted to enrich red color in control sections. Individual images of sections were cropped using ImageJ with no additional adjustment to color levels/thresholds.

### Example 7. Rodent Positron Emission Tomography-Computerized Tomography (PET-CT) Acquisition and Post Processing

Male Sprague-Dawley rats were utilized in pairs, anesthetized with inhalational isoflurane (Forane) at 3% in a carrier of 1.5-2 L/min medical oxygen and maintained at 2% isoflurane for the duration of the scan. The rats were arranged head-to-head in a Triumph Trimodality PET/CT/SPECT scanner (Gamma Medica, Northridge, Calif.). Rats were injected standard references or vehicle via a lateral tail vein catheterization at the start of Positron Emission Tomography (PET) acquisition. Dynamic PET acquisition lasted for 60 min and was followed by computed tomography (CT) for anatomic co-registration. PET data were reconstructed using a 3D-MLEM method resulting in a full width at half-maximum resolution of 1 mm. Reconstructed images were exported from the scanner in DICOM format along with an anatomic CT for rodent studies. These files were imported to PMOD (PMOD Technologies, Ltd.) and manually coregistered using six degrees of freedom.

### Example 8. Rodent PET-CT Image Analysis

Volumes of interest (VOIs) were drawn manually as spheres in brain regions guided by high resolution CT structural images and summed PET data, with a radius no less than 1 mm to minimize partial volume effects. Time-activity curves (TACs) were exported in terms of decay corrected activity per unit volume at specified time points with gradually increasing intervals. The TACs were expressed as percent injected dose per unit volume for analysis.

### Example 9. In Vivo PET Imaging

### Assay 1

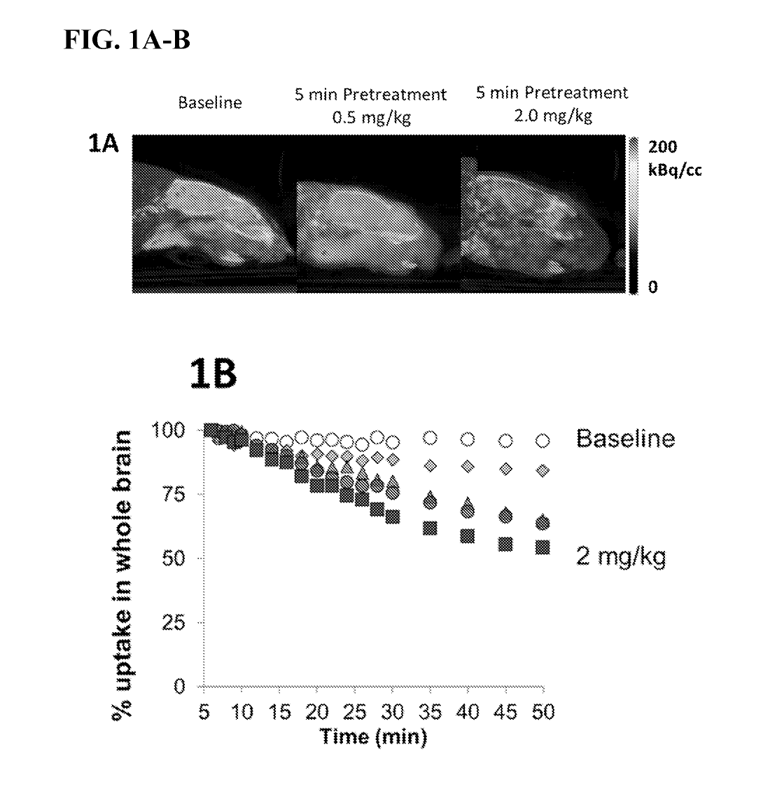

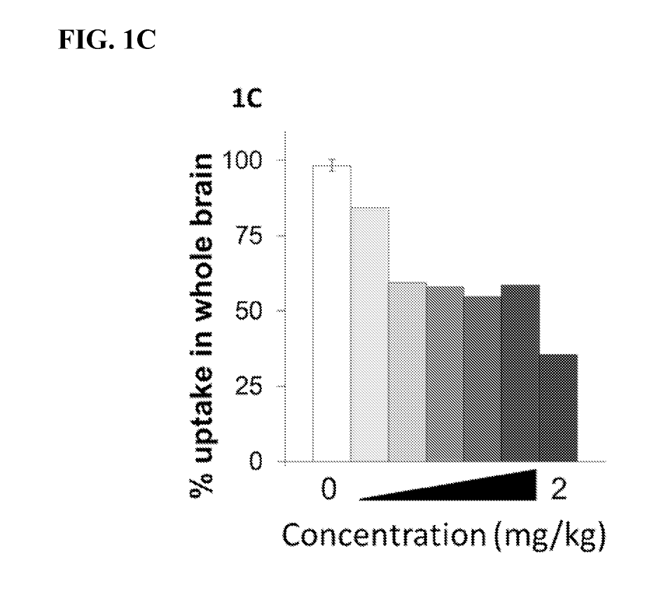

Summed PET images following injection with the ¹¹C radiolabeled (E)-3-(4-((((1r,3R,5S)-adamantan-1-ylmethyl)(methyl)amino)methyl)phenyl)-N-hydroxyacrylamide (Example 3) at baseline or after 5 minutes pretreatment with 0.5 or 2.0 mg/kg unlabeled (E)-3-(4-((((1 r,3R,5 S)-adamantan-1-ylmethyl) (methyl)amino)methyl)phenyl)-N-hydroxyacrylamide illustrate brain uptake and self-blocking, as shown in FIG. 1A. Whole-brain tracer uptake levels, normalized to uptake at 10 min, were altered by 10-40% in self-blocking experiments with 5 min pretreatment with 1.0 μg-2.0 mg/kg unlabeled (E)-3-(4-((((1r,3R,5S)-adamantan-1-ylmethyl) (methyl)amino)methyl)phenyl)-N-hydroxyacrylamide, as shown in FIGS. 1B and 1C. This quantification provides a means to measure occupancy of HDAC targets on the basis of blocked binding of the radiolabeled compound of Example 3.

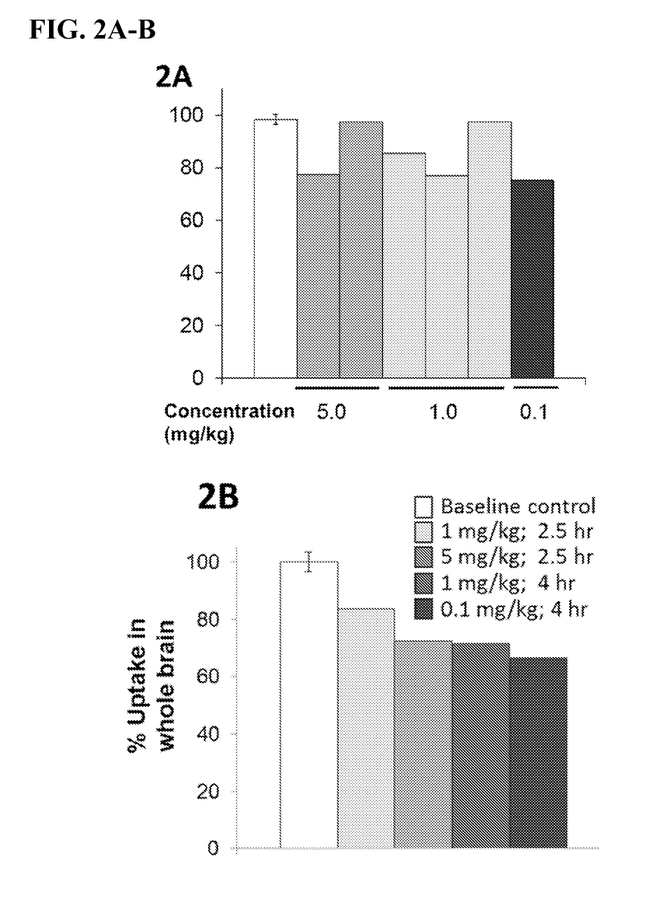

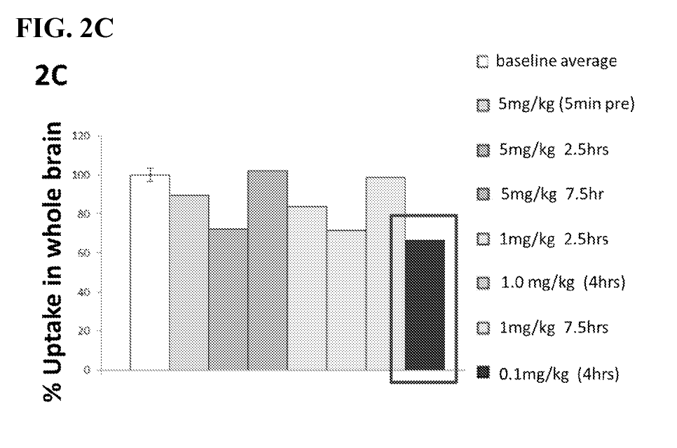

Whole brain uptake of the radiotracer of Example 3 was measured 4 h after acute systemic treatment. (E)-3-(4-((((3r,5r,7r)-adamantan-1-ylmethyl)(methyl)amino)methyl) phenyl)-N-(2-aminophenyl)acrylamide (Example 2) was found to block tracer uptake by 25% at the lowest tested dosage (0.1 mg/kg), returning to baseline by 7.5 h post relative to baseline (vehicle-blocked) PET imaging, as shown in FIG. 2A-2C.

### Assay 2

### General Conditions

Forty-nine male Sprague-Dawley rats (250-550 g, Charles River Labs) were used for the study. Animals were pair-housed on a diurnal 12:12 light/dark cycle with free access to food and water and weighed immediately prior to blocking dose administration or daily treatment. Animals were anesthetized using isoflurane/oxygen (2-3% in 1.5 L/min induction μ5-10 min.; 1.8-2.5% in 1.5 L/min for the duration of setup and PET/CT scanning, ˜80 min.) Under anesthesia, rat tails were warmed with tap water, dried, and swabbed with 70% isopropyl alcohol. A lateral tail vein was catheterized (24 GA 0.75 in, #381700, BD Biosciences), secured with tape and flushed with ˜400 μL of heparinized saline (Hospira; 2 units/mL in 0.9% saline).

Compounds were formulated for intravenous (i.v.) delivery in a vehicle containing 10% DMSO, 10% Tween 80, and 80% saline and injected in a volume of 1-2 mL/kg. Chemicals were formulated for intraperitonial (i.p.) delivery in the above vehicle or in 10% DMSO, 45% PEG400 and 45% saline and administered via injection in a volume of 2-10 mL/kg. Vehicle-treated control (i.e. baseline) animals received the same solution and injected volume without solubilized chemical. Pretreatment times >30 min were administered via i.p. injection under manual restraint. Pretreatment times <30 min were administered via i.v. or i.p. under stable anesthesia.

### Radiotracer Preparation

The radiotracer of Example 3 was prepared as described above and eluted in a solution containing 10% ethanol, 90% saline with a specific activity of 1 nCi/mMol. A dose-calibrator was used to draw radiotracer (0.3-1.3 mCi) in an injected volume of 0.5-1.1 mL. In each experiment, rats received radiotracer doses (i.v.) matched for activity and injected volume (+15%).

### Imaging and Reconstruction

Concomitant with radiotracer administration, PET data were collected for up to 60 min using dedicated small animal imaging systems: a GammaMedica Triumph PET/CT/SPECT scanner or a Siemens R4 PET scanner. PET data were reconstructed using an MLEM-3D method with 16 iterations and the following histogramming: 8×15 sec; 8×1 min; 10×2 min; 6×5 min. Attenuation correction was applied during data reconstruction using CT data (Triumph datasets) or from attenuation scans using a Germanium (Ge) 68 line source (R4).

### Image Analysis

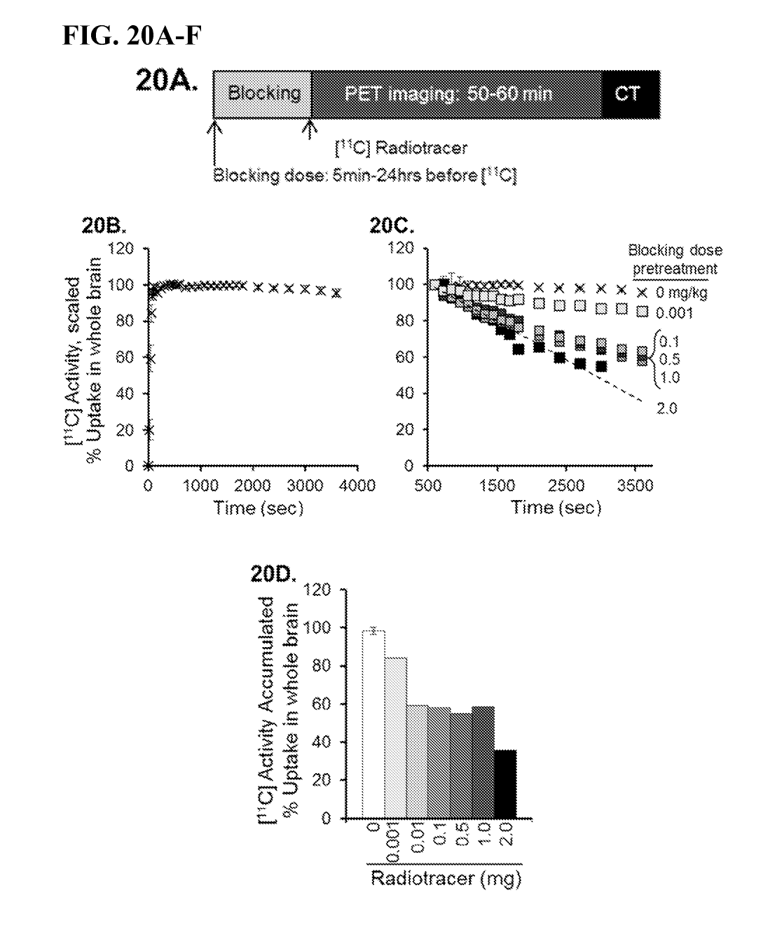

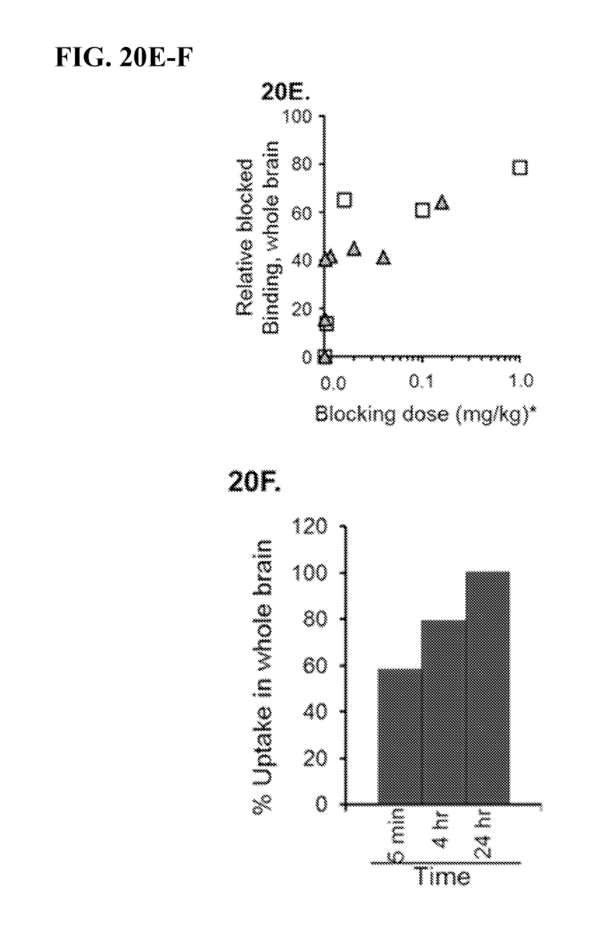

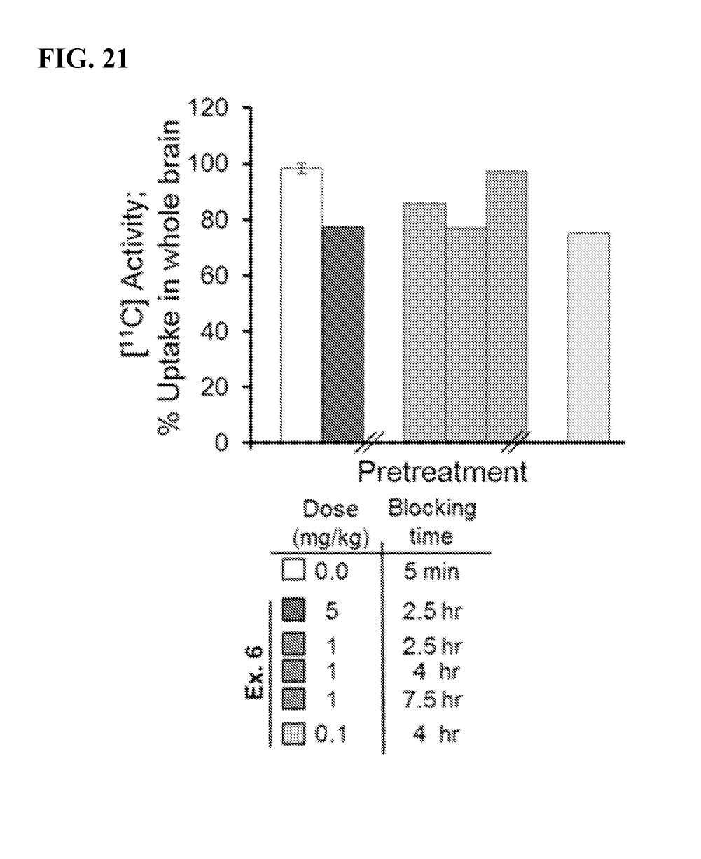

Reconstructed dynamic datasets, with the injected activity decay corrected to the start of scan, were analyzed using AMIDE software with elliptical regions of interest (ROI) placed around the whole brain, as identified from overlaid CT datasets, anatomical landmarks visible in each PET dataset (spine, lachrymal glands, nasal cavity), and radiotracer uptake intensity. Raw time-activity datasets were normalized for injected radiotracer dose (drawn dose minus post-injection residual radioactivity in syringe) and weight. To facilitate comparison between subjects, [¹¹C] signal was expressed relative to % uptake in whole brain at time=600 s. The uptake of radiotracer in rat brain is shown in FIG. 20A-20F. HDAC target binding at time=3600 s was determined for baseline and blocking conditions by the trend in the time activity curve slope from time 10 min until the end of scan. Comparison of blocked binding (n=1/condition unless noted) was made relative to the comprehensive set of baseline replicates (n=9). FIG. 21 shows the [¹¹C]percent uptake in the whole brain in the after pretreatment with the compound of Example 2.

### Example 10. Forced Swim Test & Open Field Activity Test

### General Conditions

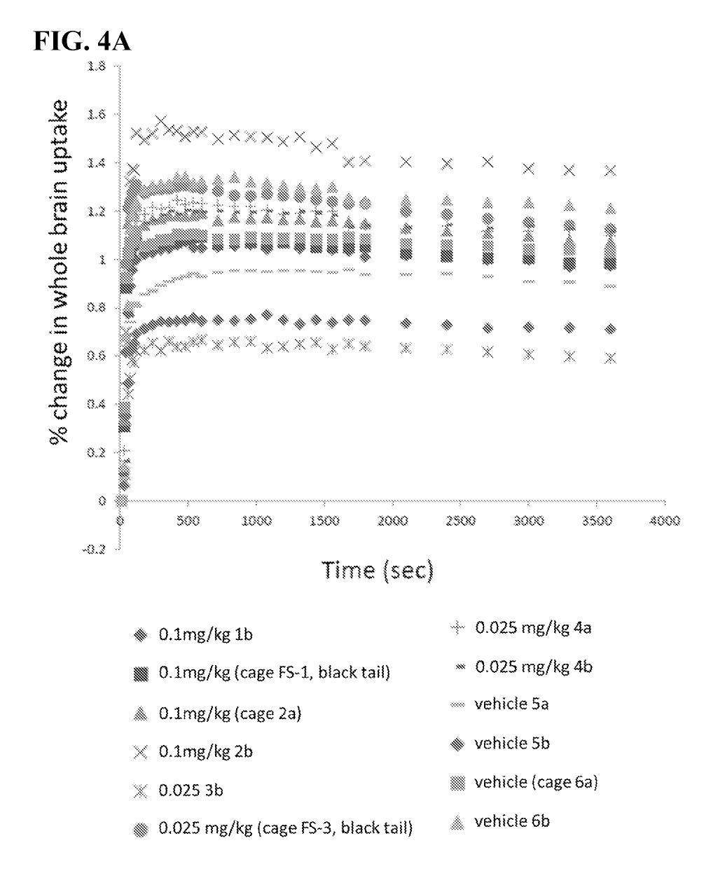

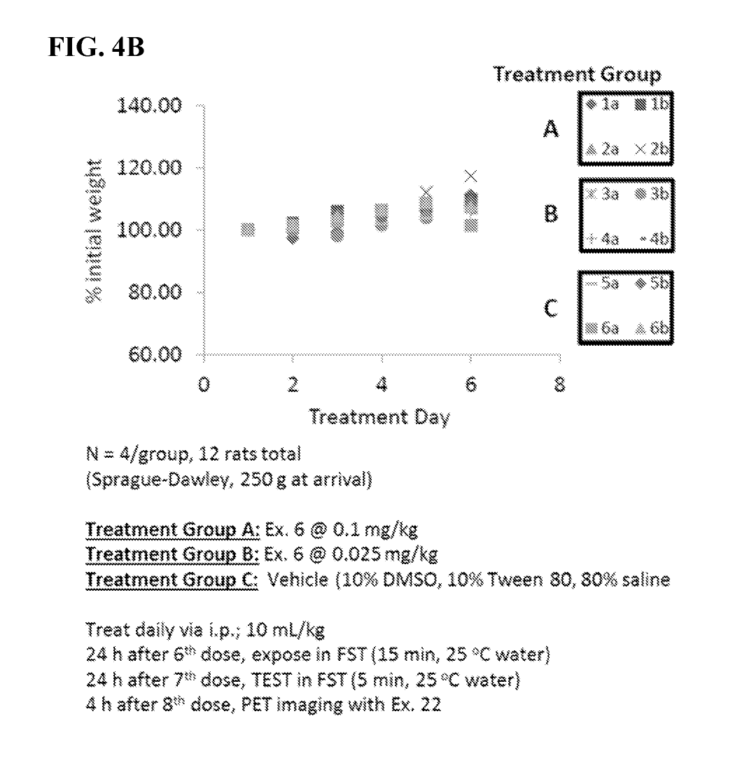

Behavioral testing was applied to a cohort of 12 rats. Following 7 days of daily i.p. treatment with i) vehicle (10% DMSO, 10% Tween 80, 80% saline; 2 mL/kg); or solubilized ii) the compound of Example 2 (0.025 mg/kg) or iii) the compound of Example 2 (0.1 mg/kg), rats were exposed to the modified forced swim test (FST). Briefly, FST exposure (15 min exposure, 25° C. tap water, 30 cm water depth) was performed 18-24 h after the previous i.p. treatment, after which rats were returned to their home cages to dry for approximately 2-3 h, then given daily i.p. treatment as before. FST testing was performed one day later (5 min test, 25° C. tap water, 30 cm water depth) with behaviors digitally recorded for subsequent analysis of immobility by a trained reviewer, blinded to treatment groups. Rats were returned to home cages and monitored without treatment for 72 h. Rats were then treated for 7 additional days with i) vehicle, ii) the compound of Example 2 (0.5 mg/kg) or iii) the compound of Example 2 (2.0 mg/kg) via daily i.p. injection; treatment assignments for each rat remained the same with a 20× increase in dose of the compound of Example 2. Animals were then evaluated for locomotor activity in an open field (60 cm square) using automated tracking software (MedAssociates). FIG. 4A shows the percent change in whole brain uptake via in vivo PET-imaging of the HDAC-selective radiolabeled compound of Example 3 representative of rats in the presence of vehicle or the compound of Example 2.

### Forced Swim Test

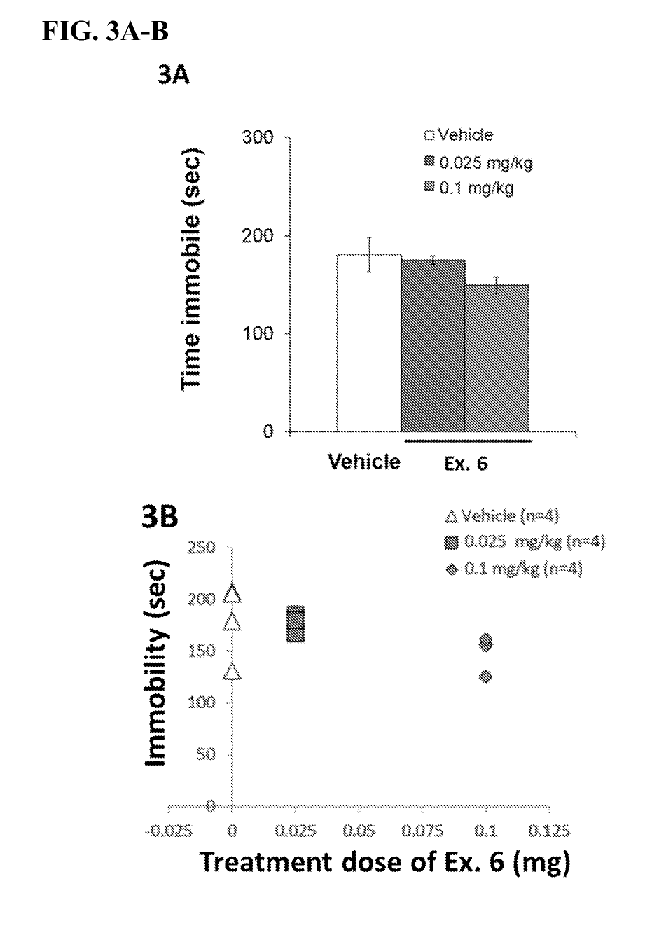

Rats were exposed to a 15 min session of forced swimming in 25° C. water (20 cm diameter cylinder, 30 cm water depth). Based on PET imaging experiments, doses of (E)-3-(4-((((3r,5r,7r)-adamantan-1-ylmethyl)(methyl)amino)methyl)phenyl)-N-(2-aminophenyl)acrylamide (Example 2) were investigated, representing a range of HDAC occupancy in the brain. Immediately after FST exposure, rats were treated via intraperitoneal (i.p.) injection with one of three treatments: i) vehicle (10% DMSO, 45% PEG400, 45% saline), ii) the compound of Example 2 at 0.1 mg/kg (˜25% HDAC occupancy) or iii) the compound of Example 2 at 0.025 mg/kg. After 1 day, rats were tested for immobility time during 5-min FST sessions which were videotaped and scored by a trained observer blinded to treatment groups. Rats treated with 0.1 mg/kg against vehicle-treated controls exhibited a 17% decrease in FST immobility scores, as shown in FIG. 3A-3B. The average weight gain of tested rats was not impacted by treatment with the compound of Example 2, as shown in FIG. 4B.

### Open Field Activity Test

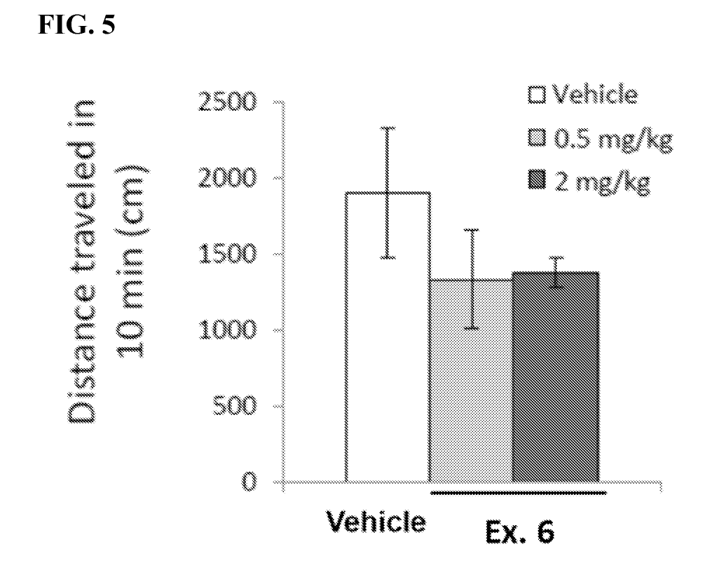

Equivalent rats, treated with 20-fold higher doses of the compound of Example 2 (relative to the FST tests) were used to confirm absence of confounding motoric effects using open field (OF) test chambers (60×60 cm box, infrared beams tracking relative position, activity, and distance traveled). Analysis of behavioral changes were relative to response in vehicle-treated controls with effects averaged over treatment groups and revealed no confounding effect in locomotor activity, as shown in FIG. 5.

### Example 11. Intraperitoneal Pharmacokinetics and Brain Distribution Assay

### General Procedures

This study was conducted at Sai Life Sciences Limited, Pune, India, in accordance with the guidelines of the Institutional Animal Ethics Committee (IAEC). The study was conducted in non-GLP conditions; however, all relevant study documents were maintained in the study file. In-house SOPs were followed for the conduct of the study.

All procedures were conducted in accordance with the guidelines provided by the Committee for the Purpose of Control and Supervision of Experiments on Animals (CPCSEA) as published in The Gazette of India, Dec. 15, 1998. Prior approval of the Institutional Animal Ethics Committee (IAEC) was obtained before initiation of the study.

Healthy male Sprague Dawley rats (8-12 weeks old) weighing between 200 to 250 g were procured from Tina Labs, Hyderabad, India. Three rats were housed in each cage. Temperature and humidity were maintained at 22±3° C. and 40-70%, respectively and illumination was controlled to give a sequence of 12 hr light and 12 hr dark cycle. Temperature and humidity were recorded by auto-controlled data logger system. All the animals were provided laboratory rodent diet (Vetcare India Pvt. Ltd, Bengaluru) ad libitum. Reverse osmosis water treated with ultraviolet light was provided ad libitum.

### General Assay Procedures

Twenty one male Sprague Dawley rats were intraperitoneally administered with a solution formulation of the compound of Example 2 prepared in 10% DMSO, 10% Tween 80, and 80% normal saline. The blood samples were collected from a set of three rats at each time point in labeled micro centrifuge tube containing K2EDTA solution as anticoagulant. Plasma samples were separated by centrifugation of whole blood and stored at below −70° C. until bioanalysis. After collection of blood samples at each time point animals, were humanely euthanized by CO₂ and the brain was collected. Brain samples were homogenized using ice-cold phosphate buffer saline (pH=7.4) and homogenates were stored at below −70° C. until analysis. Total homogenate volume was three times the brain weight. All samples were processed for analysis by protein precipitation using acetonitrile and analyzed with fit-for-purpose LC-MS/MS method (Lower Limits of Quantitation (LLOQ)=2.01 ng/mL in plasma and 1.01 ng/mL in brain). Pharmacokinetic parameters were calculated using the non-compartmental analysis tool of Phoenix WinNonlin® (Version 6.3).

### Formulation Preparation

The intraperitoneal solution formulation concentration was 0.1 mg/mL. The weighed quantity (7.02 mg) of (E)-3-(4-((((3r,5r,7r)-adamantan-1-ylmethyl)(methyl)amino)methyl)phenyl)-N-(2-aminophenyl)acrylamide was added in a labeled bottle. DMSO (7.02 mL) was added to dissolve the compound, followed by addition of Tween 80 (7.02 mL) and the solution was mixed by vortexing. Next, normal saline was added (56.16 mL) and the resulting solution was mixed by vortexing. The solution formulation was sonicated for 2 minutes to obtain a clear and transparent solution.

### Sample Collection

The blood samples were collected from set of three rats at each time point in labeled micro centrifuge tube containing K2EDTA solution as anticoagulant at time points of 0.08, 0.5, 1, 2, 4, 6, 8 & 24 hours. After blood collection, plasma was harvested by centrifugation and stored at below −70° C. prior to analysis. After collection of blood samples, animals were humanely euthanized by CO₂ and the brain was collected. Brain samples were homogenized using 2 volumes of ice-cold phosphate buffer saline (pH=7.4, assuming brain tissue density is 1 g/mL) and homogenates were stored at below −70° C. until analysis. Total final homogenate volume was three times the brain weight.

### Bioanalysis

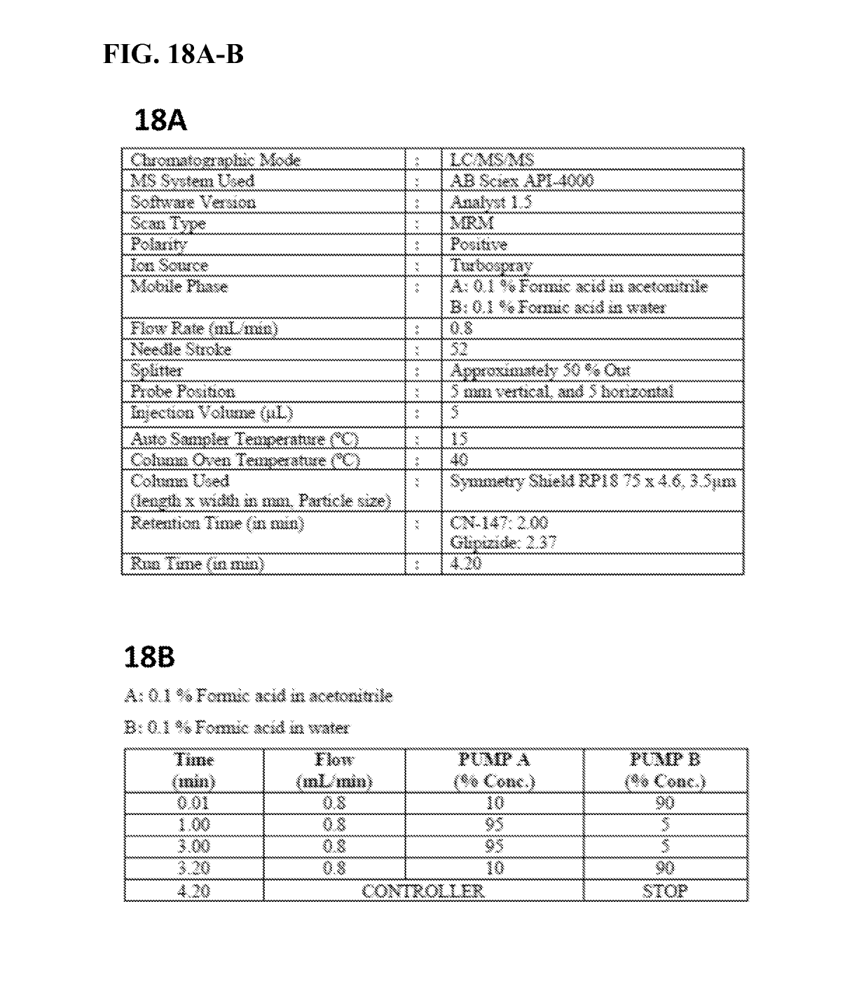

Concentrations of the compound of Example 2 in rat plasma and brain samples were analyzed with fit-for-purpose by LC-MS/MS method. A 25 μL of study sample or spiked calibration standard/quality control sample was added to individual pre-labeled micro-centrifuge tubes. An internal standard (Glipizide, 100 μL, 500 ng/mL) prepared in acetonitrile was added to each test tube (a blank was also prepared, to which 100 μL acetonitrile was added). Samples were then vortexed for 5 minutes. Samples were centrifuged for 20 minutes at a speed of 4000 rpm at 4° C. Following centrifugation, 100 L of clear supernatant was transferred a 96-well plate and analyzed using LC-MS/MS. The chromatographic and mass spectrometric conditions are shown FIG. 18A-B.

### Data Analysis

Non-Compartmental-Analysis module in Phoenix WinNonlin® (Version 6.3) was used to assess the pharmacokinetic parameters. Peak plasma concentrations (Cₘₐₓ) and time for the peak plasma concentrations (Tₘₐₓ) were the observed values. The areas under the concentration time curve (AUCₗₐₛₜ and AUC_{inf}) were calculated by linear trapezoidal rule. The terminal elimination rate constant, ke was determined by regression analysis of the linear terminal portion of the log plasma concentration time curve. The terminal half-life (T_{1/2}) was estimated as 0.693/ke; CL=Dose/AUC_{inf}; V_{z}=MRT×CL; MRT=AUMC_{inf}/AUC_{inf}.

### Intraperitoneal Assay Results

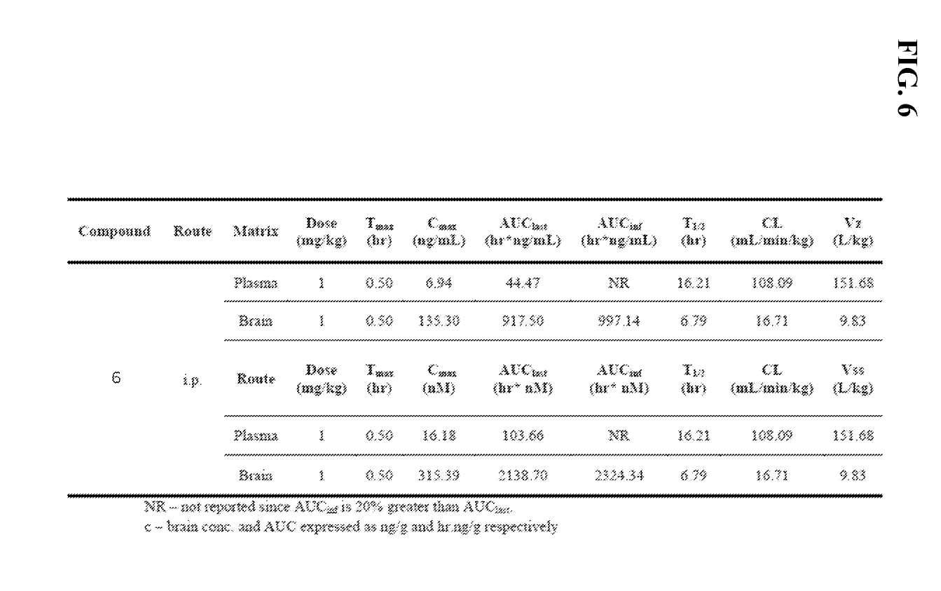

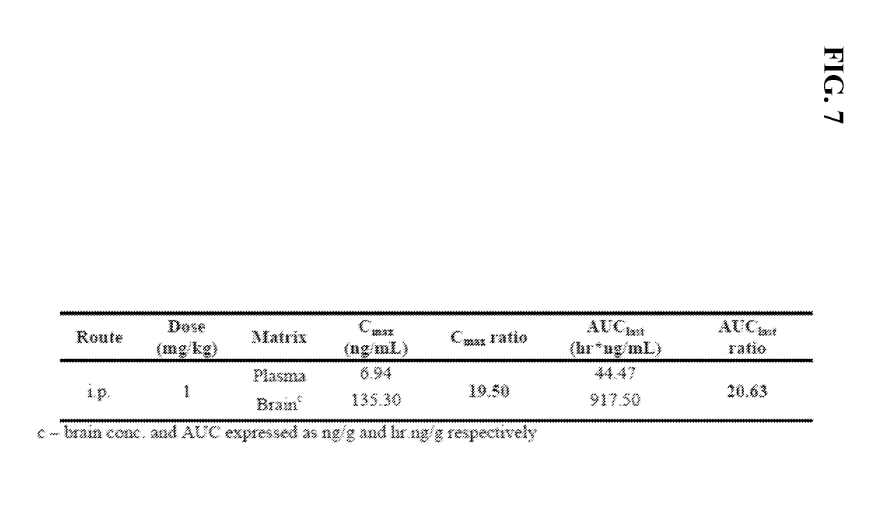

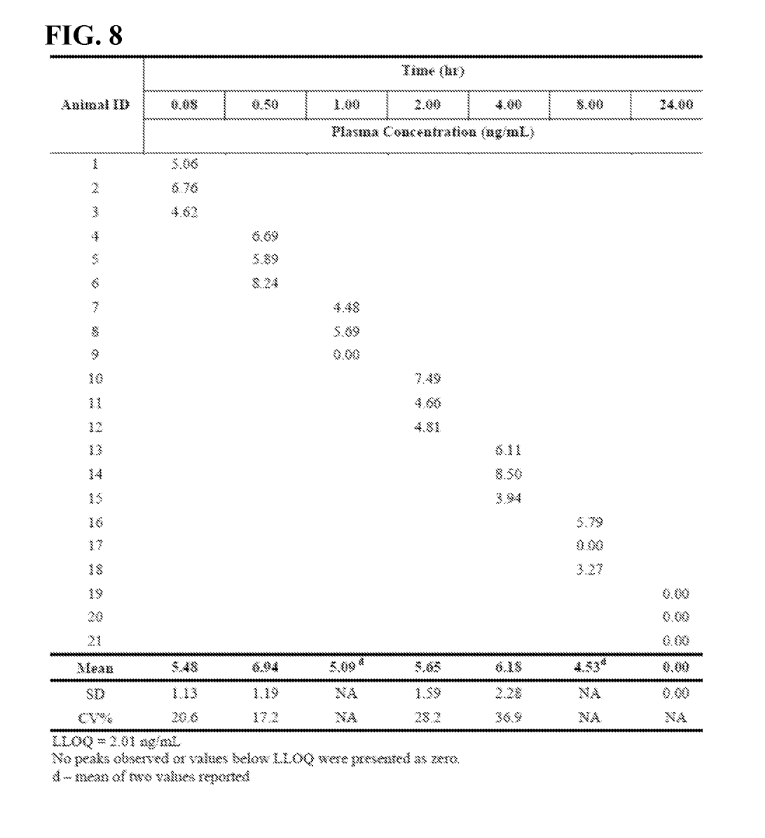

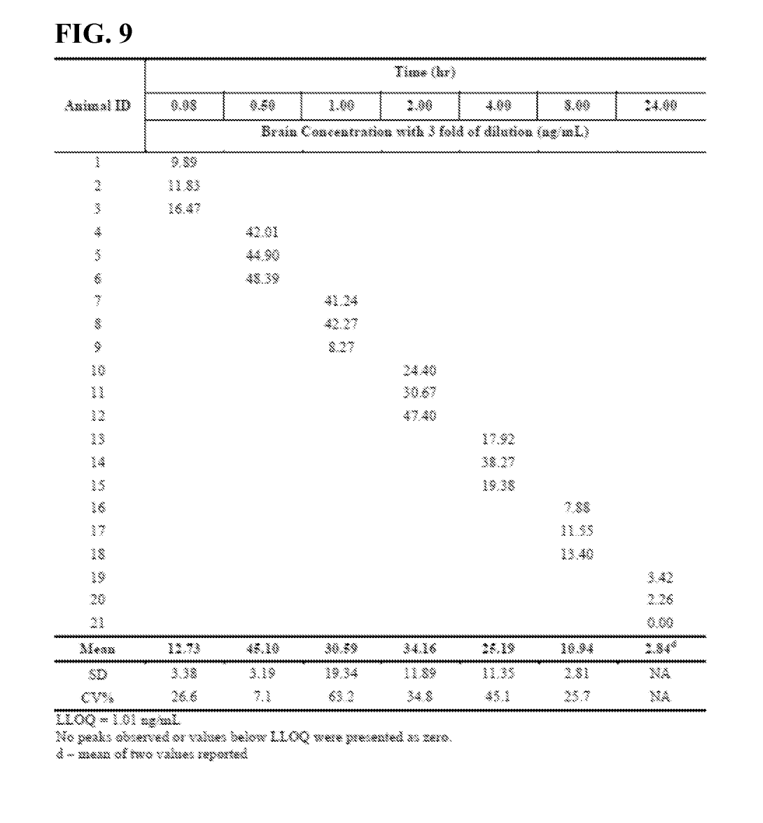

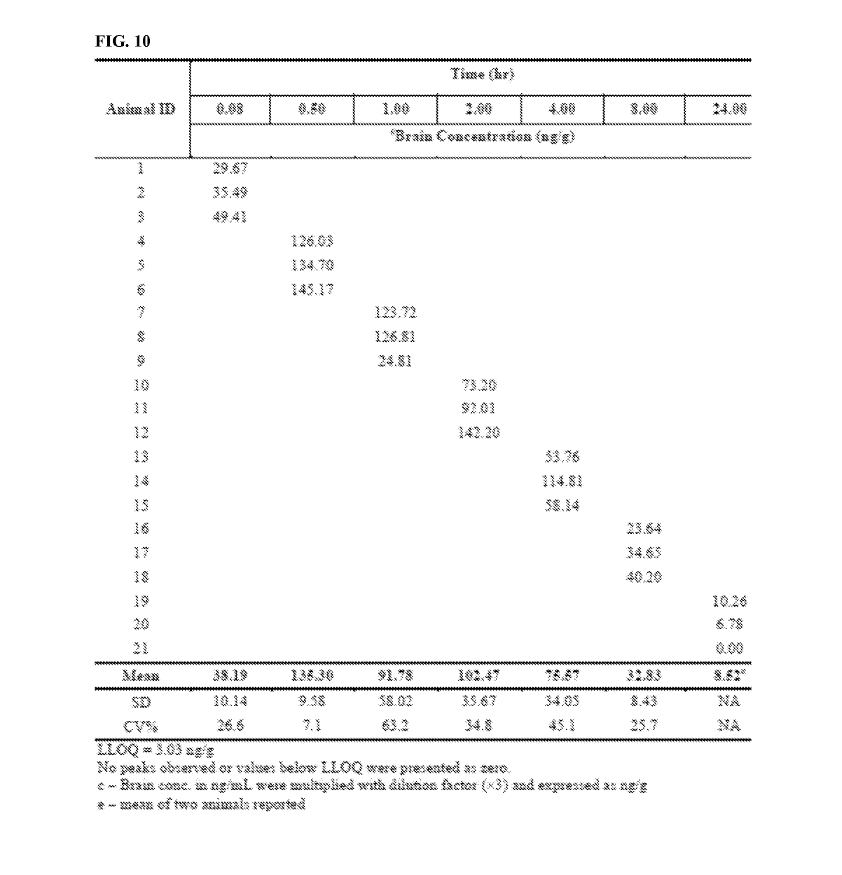

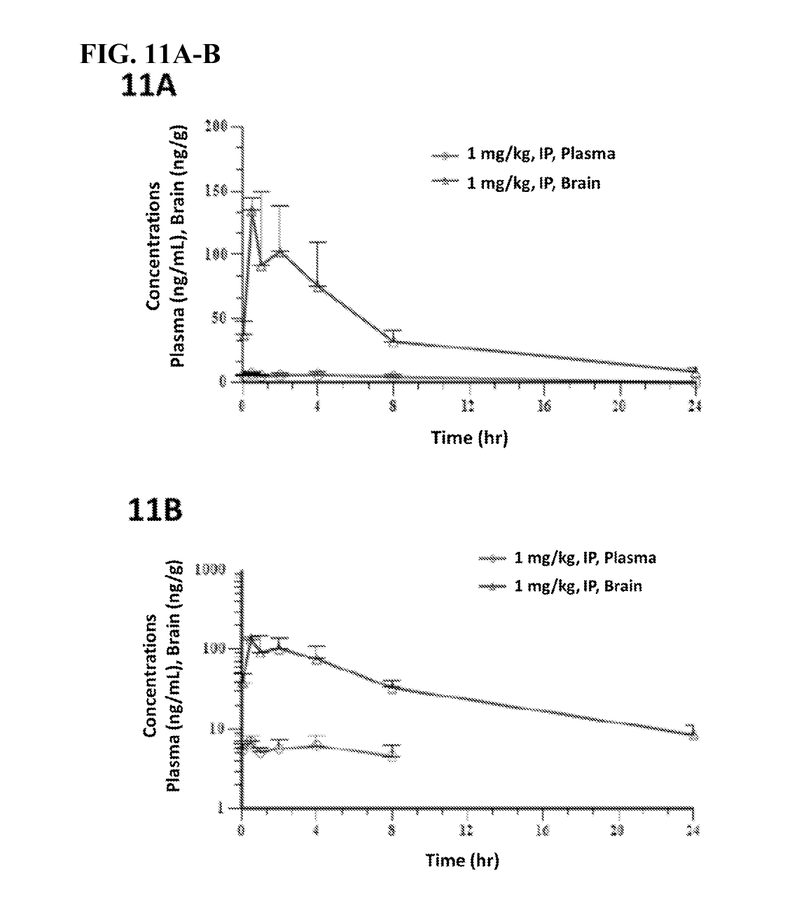

Following a single intraperitoneal (i.p.) dose administration of the compound of Example 2 to male Sprague Dawley rats at 1 mg/kg dose, plasma and brain concentrations were quantifiable up to 8 h (2 animals) and 24 h (2 animals), respectively, with a Tₘₐₓ of 0.5 hr. Brain-to-plasma exposure ratio was 20.63. Pharmacokinetic parameters for the compound of Example 2 are shown in FIG. 6-11B.

### Example 12. Intraveneous Pharmacokinetics and Brain Distribution Assay

### General Procedures

The study was conducted at Sai Life Sciences Limited, Pune, India, in accordance with the guidelines of the Institutional Animal Ethics Committee (IAEC). The study was conducted in non-GLP conditions; however, all relevant study documents were maintained in the study file. In-house SOPs were followed for the conduct of the study.

All procedures were conducted in accordance with the guidelines provided by the Committee for the Purpose of Control and Supervision of Experiments on Animals (CPCSEA) as published in The Gazette of India, Dec. 15, 1998. Prior approval of the Institutional Animal Ethics Committee (IAEC) was obtained before initiation of the study.

Healthy male Sprague Dawley rats (8-12 weeks old) weighing between 200 to 250 g were procured from In vivo Bioscience, Bengluru, India. Three rats were housed in each cage. Temperature and humidity were maintained at 22±3° C. and 40-70%, respectively and illumination was controlled to give a sequence of 12 h light and 12 h dark cycle. Temperature and humidity were recorded by auto-controlled data logger system. All the animals were provided laboratory rodent diet (Vetcare India Pvt. Ltd, Bengaluru) ad libitum. Reverse osmosis water treated with ultraviolet light was provided ad libitum.

### General Assay Procedures

Sprague Dawley rats were intravenously administered a solution formulation of the compound of Example 2 prepared in 10% DMSO, 10% Tween 80, 80% normal saline. The blood samples were collected from a set of three rats at each time point in labeled micro centrifuge tube containing K2EDTA solution as anticoagulant. Plasma samples were separated by centrifugation of whole blood and stored at below −70° C. until bioanalysis. After collection of blood samples at each time point animals were humanely euthanized by CO2 and the brain was collected. Brain samples were homogenized using ice-cold phosphate buffer saline (pH=7.4) and homogenates were stored at below −70° C. until analysis. Total homogenate volume was three times the brain weight. All samples were processed for analysis by protein precipitation using acetonitrile and analyzed with fit-for-purpose LC-MS/MS method (LLOQ=1.01 ng/mL in plasma and brain). Pharmacokinetic parameters were calculated using the non-compartmental analysis tool of Phoenix WinNonlin® (Version 6.3).

### Formulation Preparation

The compound of Example 2 (5.89 mg) was added in a labeled bottle, followed by DMSO (2.945 mL), Tween 80 (2.945 mL), and normal saline (23.50 mL), and the resulting mixture was mixed by vortexing after each addition. The solution formulation was sonicated for 2 minutes to obtain a clear solution. After preparation of formulation, 200 μL samples were aliquoted for analysis. The formulation was analyzed and the concentrations was found to be 0.18 mg/mL. The formulations were found to be within the acceptance criteria (in-house acceptance criteria is +20% from the nominal value). All formulations were freshly prepared prior to dosing.

### Sample Collection

The blood samples were collected from set of three rats at each time point in labeled micro centrifuge tube containing K2EDTA solution as anticoagulant at time points of 0.08, 0.5, 1, 2, 4, 8 & 24 h. After collection of blood, plasma was harvested by centrifugation and stored at below −70° C. prior to analysis. After collection of blood samples, animals were humanely euthanized by CO₂ and the brain was collected. Brain samples were homogenized using ice-cold phosphate buffer saline (pH=7.4) and homogenates were stored at below −70° C. until analysis. Total homogenate volume was three times the brain weight.

### Bioanalysis

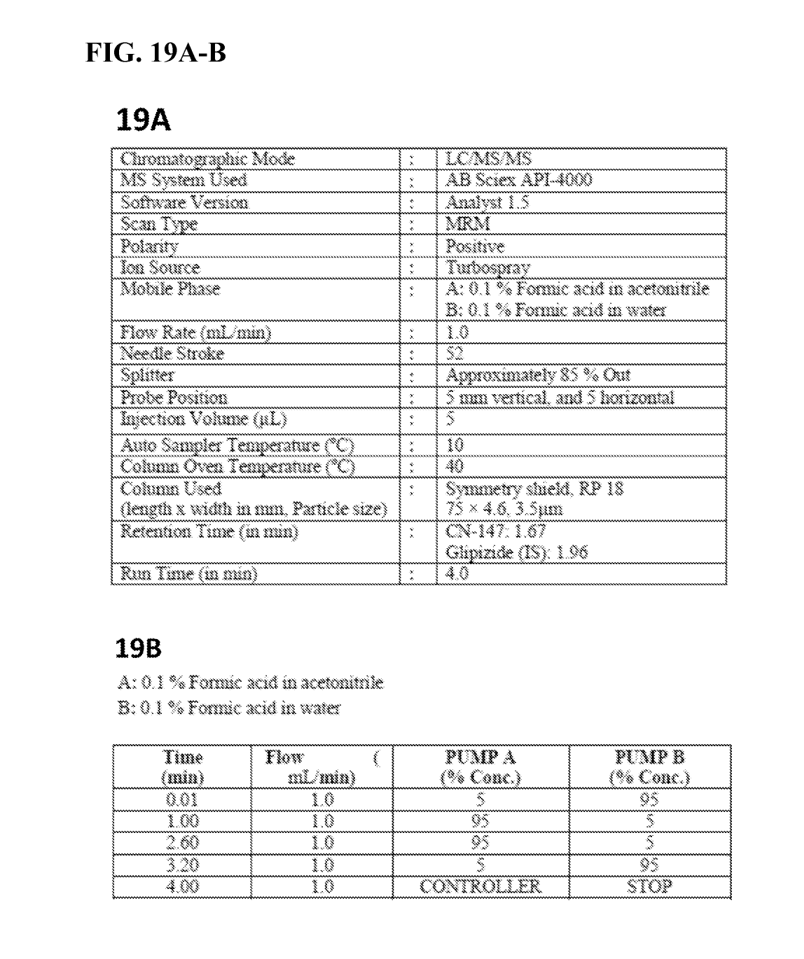

Concentrations of the compound of Example 2 in rat plasma and brain samples were analyzed with fit-for-purpose by LC-MS/MS method. A study sample (25 μL) (Dilution Factor applied to few samples) or spiked calibration standard was added to individual pre-labeled micro-centrifuge tubes followed by 100 μL of internal standard (Glipizide, 500 ng/mL) prepared in acetonitrile (a blank was also prepared, to which 100 L acetonitrile was added). Samples were vortexed for 5 minutes. Samples were the centrifuged for 10 minutes at a speed of 4000 rpm at 4° C. Following centrifugation, 100 L of clear supernatant was transferred to a 96-well plate and analyzed using LC-MS/MS. The chromatographic and mass spectrometric conditions are shown in FIG. 19A-B.

### Data Analysis

Non-Compartmental-Analysis module in Phoenix WinNonlin® (Version 6.3) was used to assess the pharmacokinetic parameters. Peak plasma concentrations (Cmax) and time for the peak plasma concentrations (Tmax) were the observed values. The areas under the concentration time curve (AUClast and AUCinf) were calculated by linear trapezoidal rule. The terminal elimination rate constant, ke, was determined by regression analysis of the linear terminal portion of the log plasma concentration-time curve. The terminal half-life (T½) was estimated to be 0.693/ke; CL=Dose/AUCinf; Vss=MRT×CLi.v.; MRT=AUMCinf/AUCinf.

### Assay Results

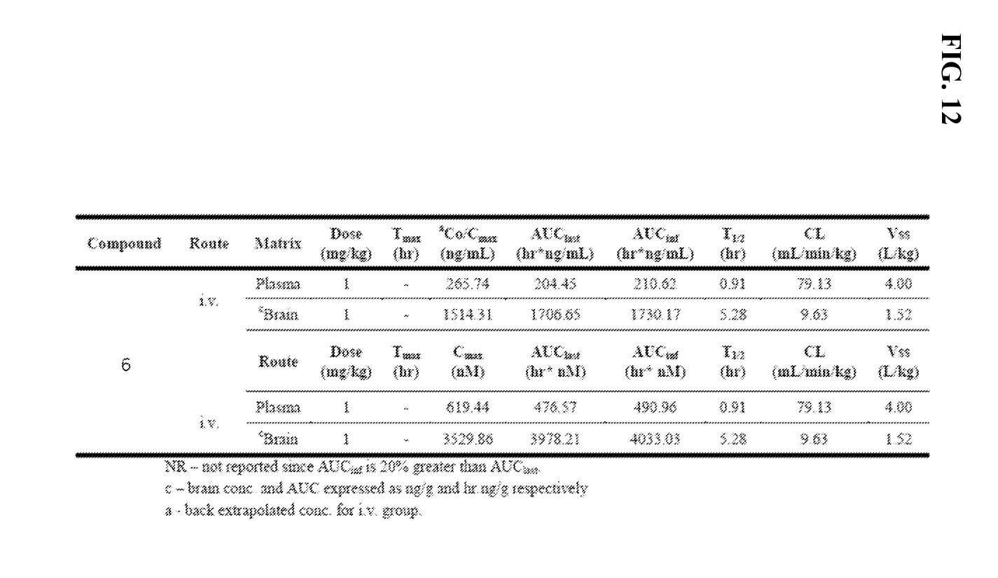

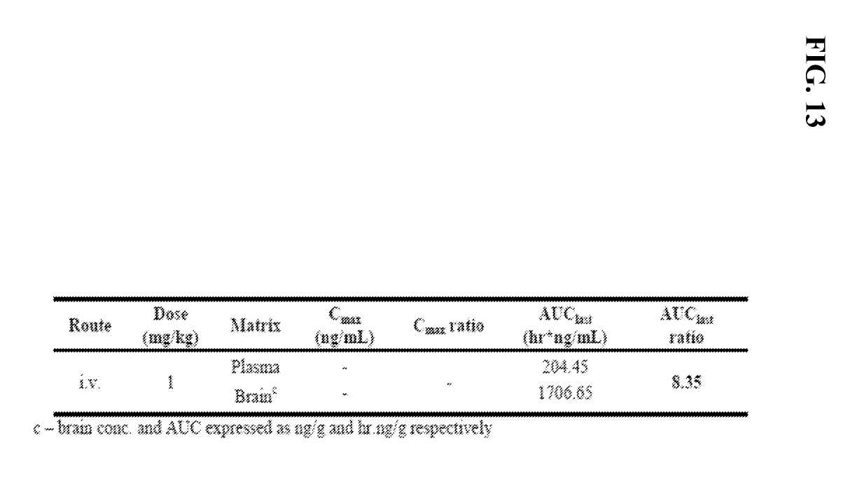

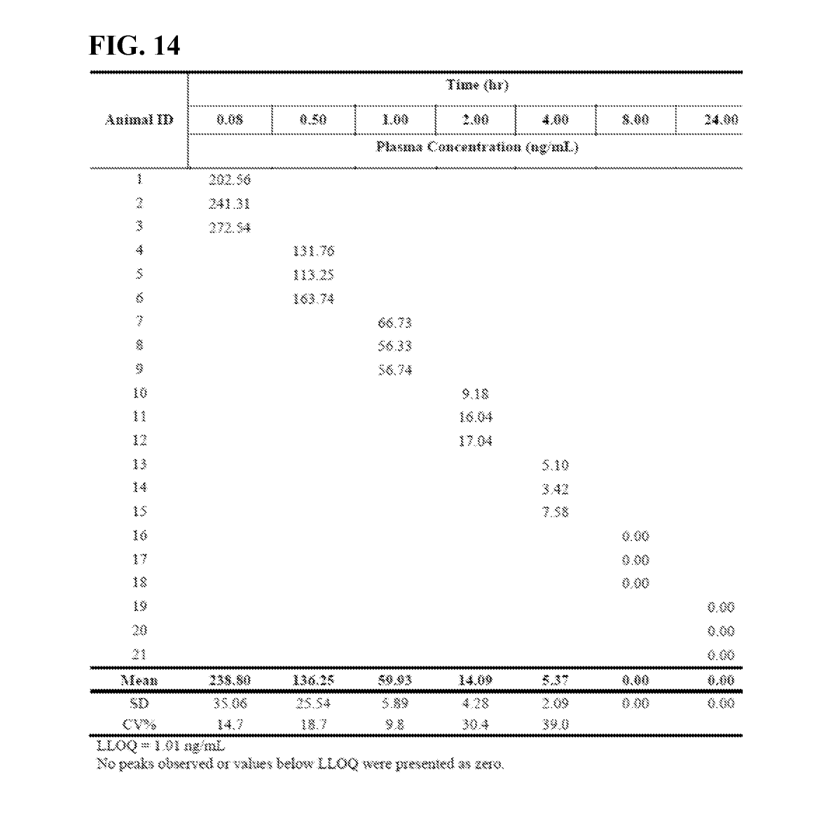

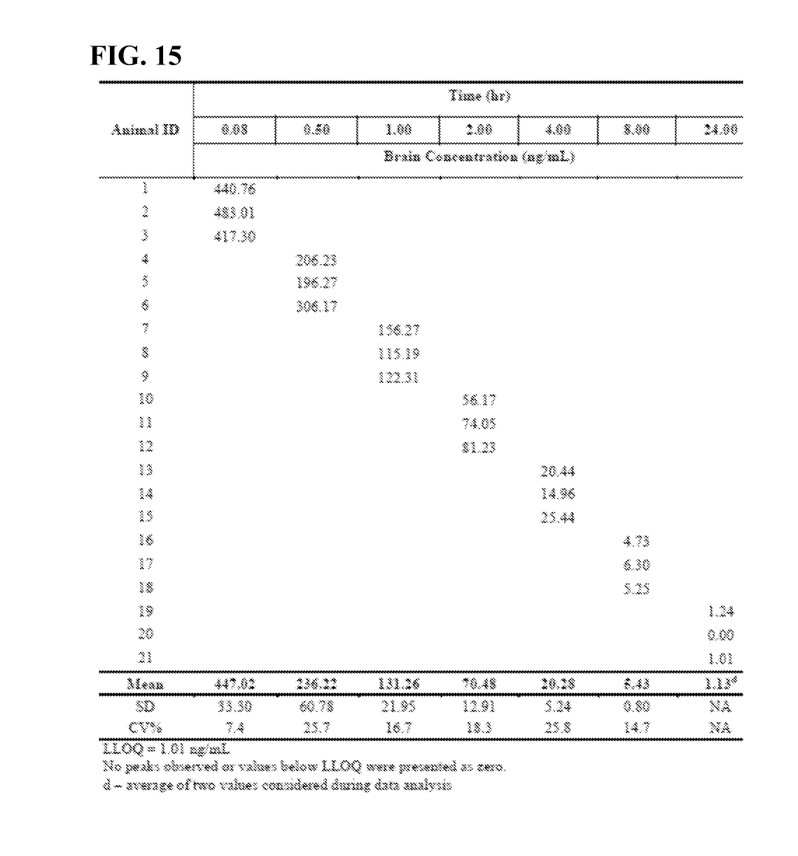

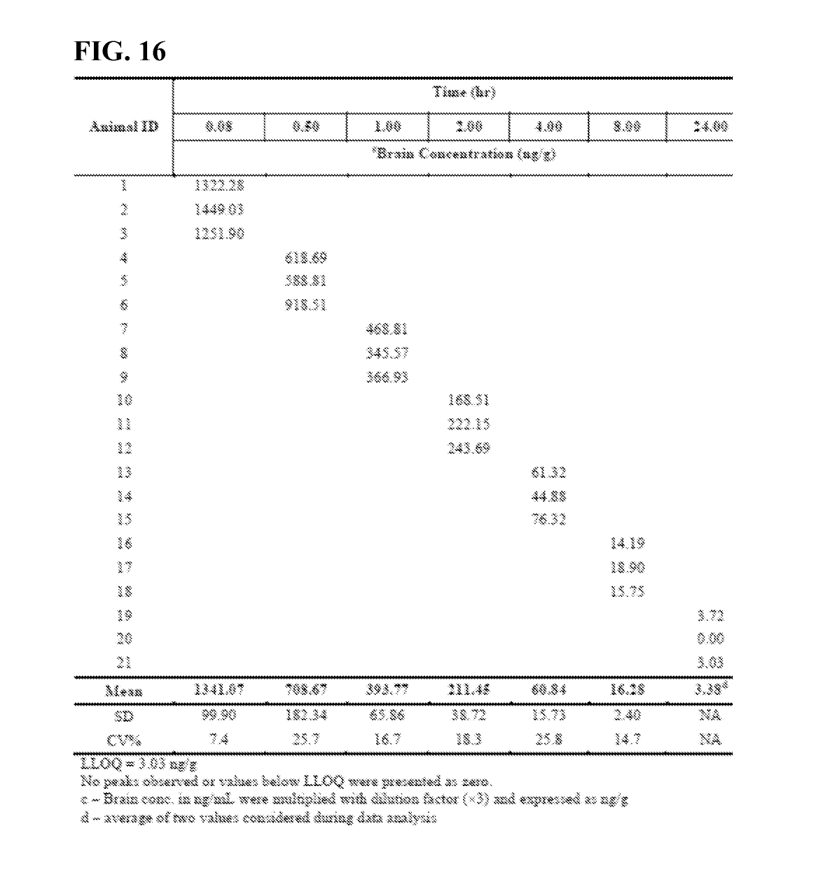

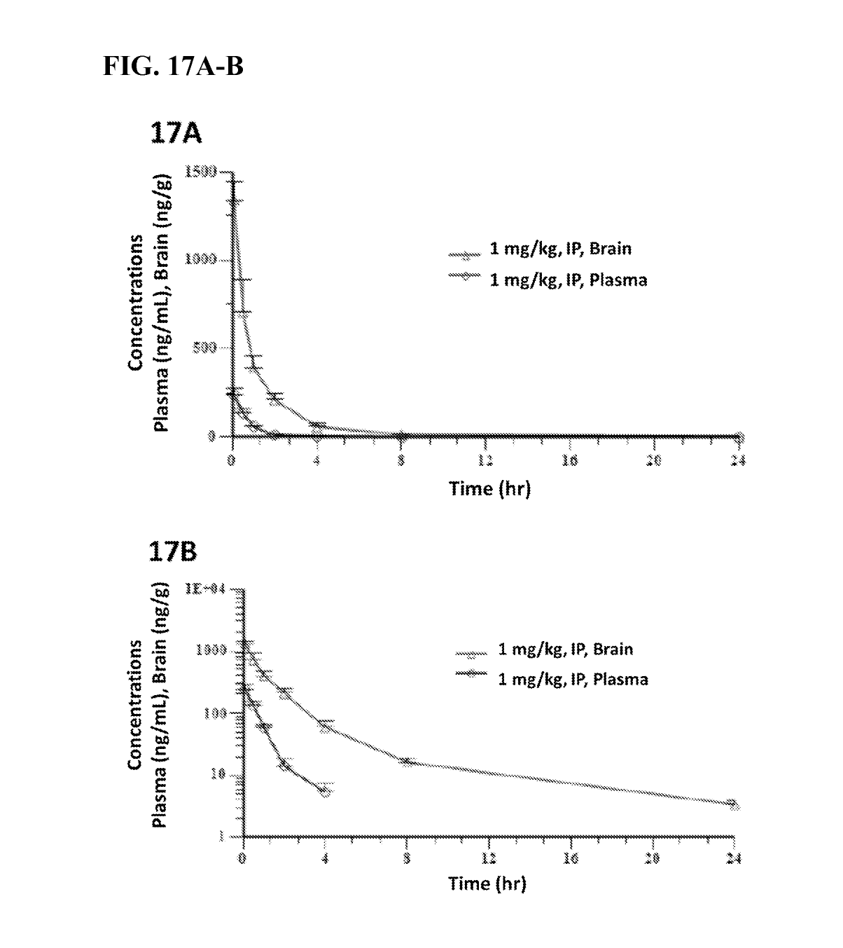

Following a single intravenous administration of the compound of Example 2 to male Sprague Dawley rats at 1 mg/kg dose, the compound exhibited plasma clearance (79 mL/min/kg; the normal liver blood flow in rat=55 mL/min/kg) with elimination half-life of 0.91 h. The Vₛₛ was 6-fold greater than the normal volume of total body water (0.7 L/kg) and the brain-to-plasma exposure ratio was 8.35. Intravenous pharmacokinetic parameters of the compound of Example 2 are shown in FIG. 12-17B.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of Formula (I): 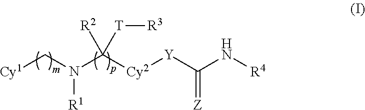
or a pharmaceutically acceptable salt thereof, wherein:
each T is independently absent or a C₁₋₄ alkylene;
Y is absent, C₁₋₄ alkylene, or C₂₋₄ alkenylene;
Z is selected from the group consisting of O, S, and NR^{b};
R¹ is selected from the group consisting of H, C₁₋₆ alkyl, C₁₋₆ haloalkyl, C₂₋₆ alkenyl, C₂₋₆ alkynyl, and OR^{a}; wherein said C₁₋₆ alkyl, C₂₋₆ alkenyl, and C₂₋₆ alkynyl are each optionally substituted with 1, 2, 3, or 4 independently selected R⁶ groups;
each R² is independently selected from the group consisting of absent, H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, and —S(═O)₂R^{c}; wherein said C₁₋₆ alkyl, C₂₋₆ alkenyl, and C₂₋₆ alkynyl are each optionally substituted with 1, 2, 3, or 4 independently selected R⁶ groups;
each R³ is independently selected from the group consisting of H, C₁₋₆ alkyl, C₁₋₆ haloalkyl, C₂₋₆ alkenyl, C₂₋₆ alkynyl, CN, NO₂, OR^{a}, SR^{a}, —NR^{a}R^{b}, —S(═O)R^{c}, —S(═O)₂R^{c}, (═O), (═S), and (═NR^{b}); wherein said C₁₋₆ alkyl, C₂₋₆ alkenyl, and C₂₋₆ alkynyl are each optionally substituted with 1, 2, 3, or 4 independently selected R⁶ groups;
R⁴ is selected from the group consisting of H, C₁₋₆ alkyl, C₁₋₆ haloalkyl, C₂₋₆ alkenyl, C₂₋₆ alkynyl, OR^{a}, and Cy³; wherein said C₁₋₆ alkyl, C₂₋₆ alkenyl, and C₂₋₆ alkynyl are each optionally substituted with 1, 2, 3, or 4 independently selected R⁶ groups;
each R⁶ is independently selected from OH, NO₂, CN, halo, C₁₋₃ alkyl, C₂₋₄ alkenyl, C₂₋₄ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₄ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
each R^{a}, R^{b}, and R^{c} is independently H or C₁₋₆ alkyl;
Cy¹ is selected from the group consisting of C₃₋₁₀ cycloalkyl, 4-10 membered heterocycloalkyl, C₆₋₁₀ aryl, and 4-10 membered heteroaryl; wherein said C₃₋₁₀ cycloalkyl, 4-10 membered heterocycloalkyl, C₆₋₁₀ aryl, and 4-10 membered heteroaryl are each optionally substituted by 1, 2, 3, or 4 substituents independently selected from C₁₋₆ alkyl, C₁₋₆ haloalkyl, C₁₋₄ alkoxy, halo, OH, and CN;
Cy² is selected from the group consisting of C₆₋₁₀ aryl, C₃₋₁₀ cycloalkyl, 4-10 membered heteroaryl, and 4-10 membered heterocycloalkyl; wherein said C₆₋₁₀ aryl, C₃₋₁₀ cycloalkyl, 4-10 membered heteroaryl, and 4-10 membered heterocycloalkyl are each optionally substituted by 1, 2, 3, or 4 substituents independently selected from C₁₋₆ alkyl, C₁₋₆ haloalkyl, C₁₋₄ alkoxy, halo, OH, and CN;
Cy³ is selected from the group consisting of C₃₋₁₀ cycloalkyl, 4-10 membered heterocycloalkyl, C₆₋₁₀ aryl, and 4-10 membered heteroaryl; wherein said C₃₋₁₀ cycloalkyl, 4-10 membered heterocycloalkyl, C₆₋₁₀ aryl, and 4-10 membered heteroaryl are each substituted by 1, 2, 3, or 4 substituents independently selected from halo, OH, CN, C₁₋₆ alkyl, C₁₋₆ haloalkyl, C₁₋₄ alkoxy, C₆₋₁₀ aryl, 4-10 membered heteroaryl, amino, C₁₋₃ alkylamino, di(C₁₋₃ alkyl)amino, carbamyl, C₁₋₃ alkylcarbamyl, di(C₁₋₃ alkyl)carbamyl, C₁₋₃ alkylcarbonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
m is 0, 1, 2, 3, 4, 5, or 6; and
p is 0, 1, 2, 3, 4, 5, or 6,
with the proviso that the compound of Formula (I) is not selected from the group consisting of:

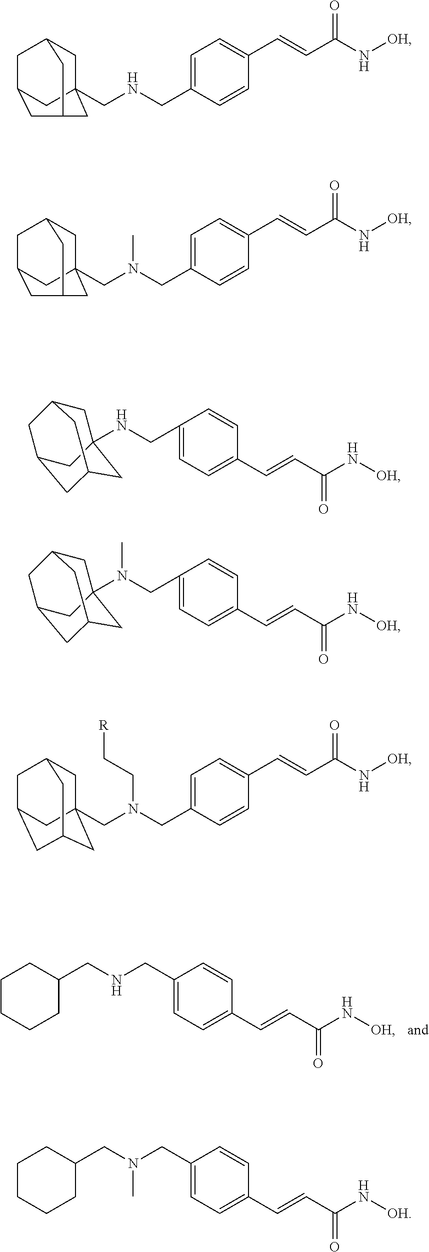

2. The compound of claim 1, wherein:
m is 0, 1, or 2; and
p is 1.

3.-5. (canceled)

6. The compound of claim 1, wherein Y is absent or C₂₋₄ alkenylene.

7.-10. (canceled)

11. The compound of claim 1, wherein Z is O.

12. The compound of claim 1, wherein R¹ is selected from the group consisting of H, C₁₋₆ alkyl, and C₁₋₆ haloalkyl.

13.-16. (canceled)

17. The compound of claim 1, wherein each R² is independently selected from the group consisting of absent, H, C₁₋₆ alkyl, and C₁₋₆ haloalkyl.

18. (canceled)

19. The compound of claim 1, wherein each R³ is independently selected from the group consisting of H, C₁₋₆ alkyl, C₁₋₆ haloalkyl, OR^{a}, (═O), (═S), and (═NR^{b}).

20.-23. (canceled)

24. The compound of claim 1, wherein R⁴ is selected from the group consisting of H, OH, or Cy³.

25.-26. (canceled)

27. The compound of claim 1, wherein R⁴ is a C₆₋₁₀ aryl or a 4-10 membered heteroaryl ring.

28.-39. (canceled)

40. The compound of claim 1, wherein each R⁶ is independently selected from the group consisting of OH, NO₂, CN, halo, and C₁₋₃ alkyl.

41. The compound of claim 1, wherein Cy¹ is a C₃₋₁₀ cycloalkyl or a 4-10 membered heterocycloalkyl.

42.-45. (canceled)

46. The compound of claim 1, wherein Cy² is C₆₋₁₀ aryl or 4-10 membered heterocycloalkyl.

47.-60. (canceled)

61. The compound of claim 1, wherein the compound of Formula (I) is selected from the group consisting of: 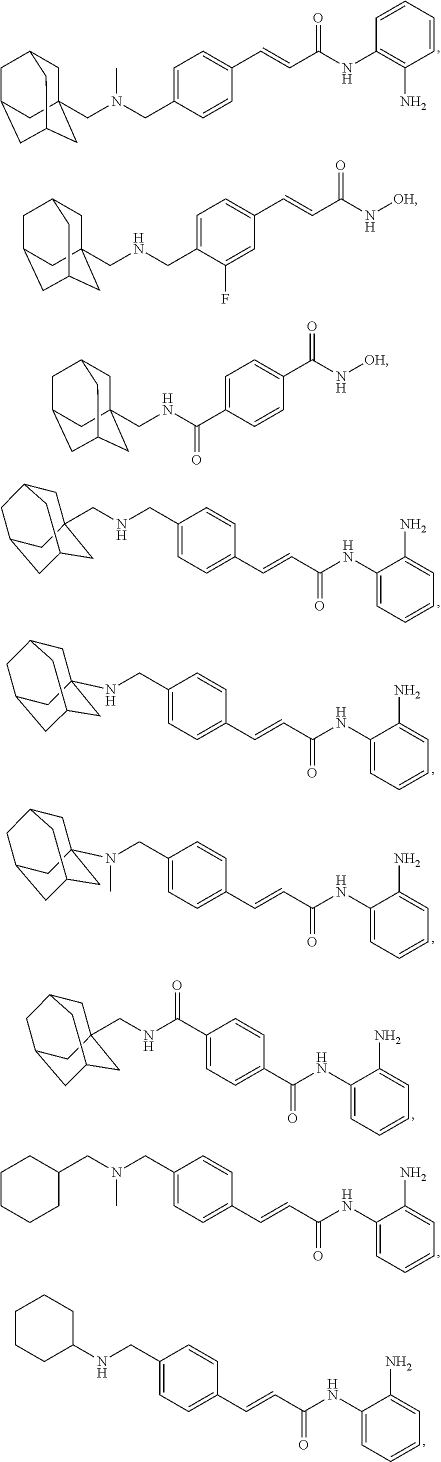 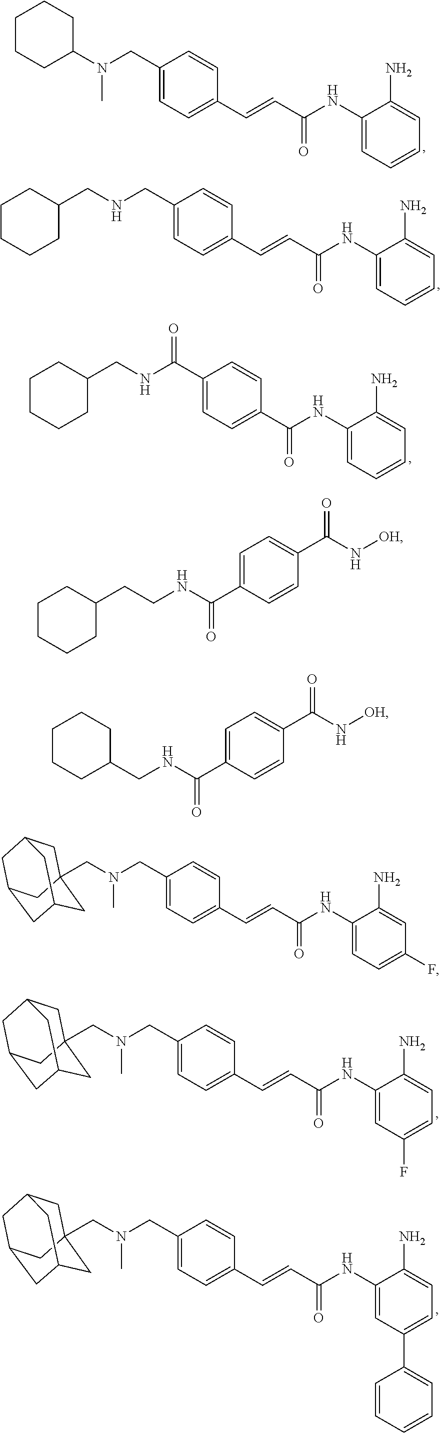 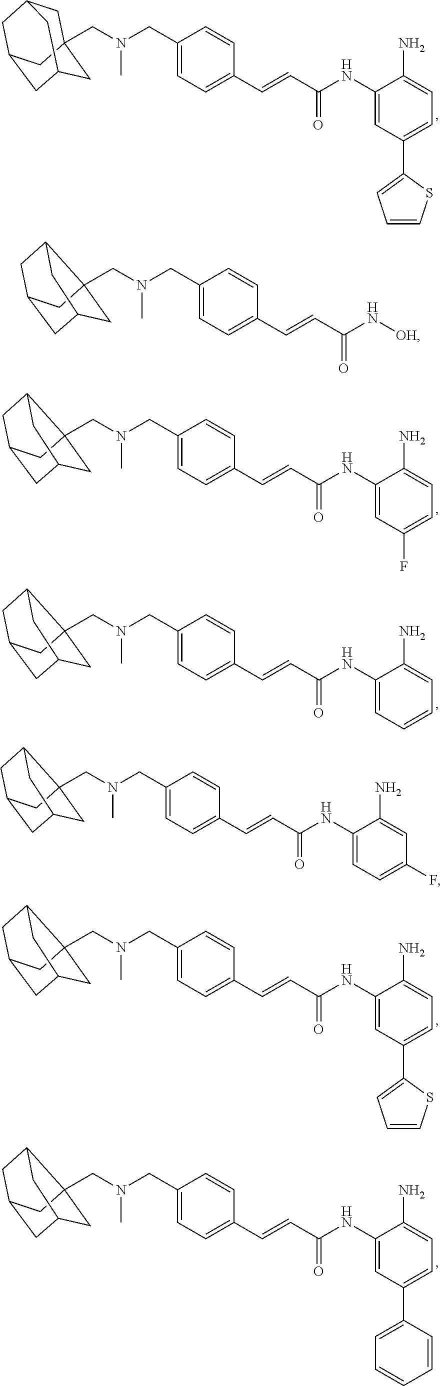 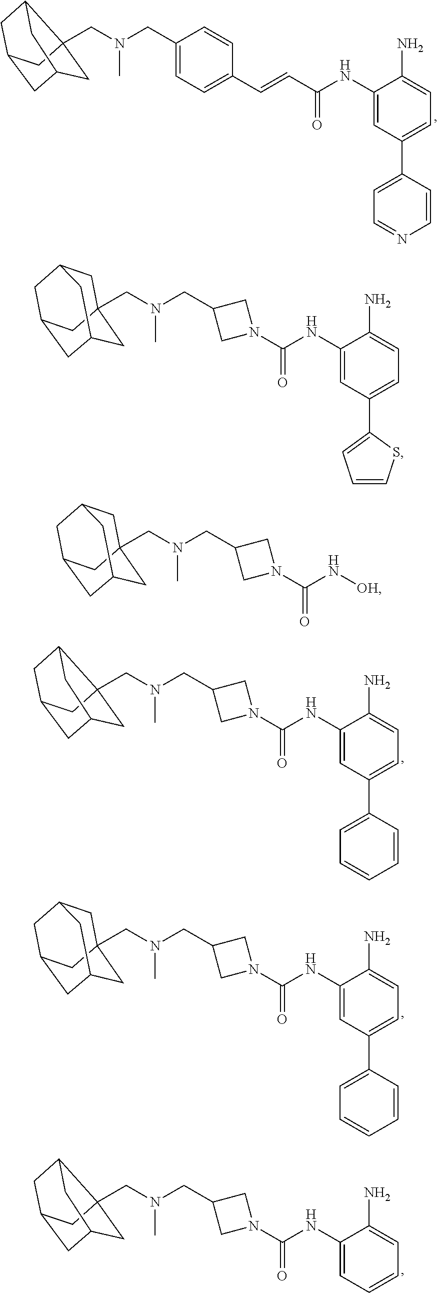 [SMILES: CN(Cc1ccc(/C=C/C(=O)Nc2cc(-c3ccncc3)ccc2N)cc1)CC12CC3CC(CC1C3)C2 ; CN(CC1CN(C(=O)Nc2cc(-c3cccs3)ccc2N)C1)CC12CC3CC(CC(C3)C1)C2 ; CN(CC1CN(C(=O)NO)C1)CC12CC3CC(CC(C3)C1)C2 ; CN(CC1CN(C(=O)Nc2cc(-c3ccccc3)ccc2N)C1)CC12CC3CC(CC1C3)C2 ; CN(CC1CN(C(=O)Nc2cc(-c3ccccc3)ccc2N)C1)CC12CC3CC(CC(C3)C1)C2 ; CN(CC1CN(C(=O)Nc2ccccc2N)C1)CC12CC3CC(CC1C3)C2] 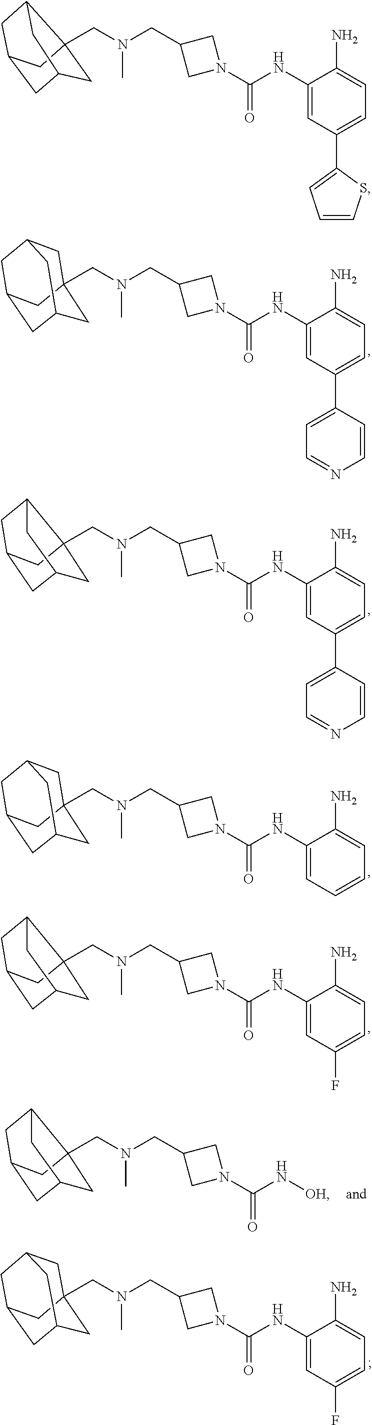
or a pharmaceutically acceptable salt thereof.

62. The compound of claim 1, wherein the compound of Formula (I) is: 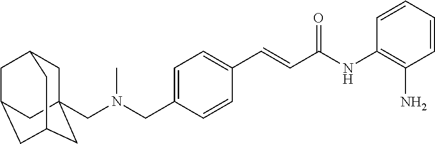 [SMILES: CN(Cc1ccc(/C=C/C(=O)Nc2ccccc2N)cc1)CC12CC3CC(CC(C3)C1)C2]
or a pharmaceutically acceptable salt thereof.

63. A pharmaceutical composition comprising a compound of claim 1, or a pharmaceutically acceptable salt thereof, and at least one pharmaceutically acceptable carrier.

64. (canceled)

65. A method of inhibiting an activity of a histone deacetylase (HDAC) enzyme, comprising contacting said HDAC enzyme with a compound of claim 1, or a pharmaceutically acceptable salt thereof.

66.-74. (canceled)

75. A method of treating a disease in a patient in need thereof, said method comprising administering to said patient a therapeutically effective amount of a compound of claim 1, or a pharmaceutically acceptable salt thereof, wherein said disease is selected from the group consisting of cancer, a disease of the central nervous system, and an inflammatory autoimmune disease.

76.-107. (canceled)

108. A method of treating a cancer in a patient, the method comprising:
i) identifying the cancer as being associated with abnormal activity or abnormal expression of a histone deacetylase (HDAC); and
ii) if the cancer is identified as being associated with abnormal activity of a histone deacetylase (HDAC), then administering to the patient a therapeutically effective amount of a compound of claim 1, or a pharmaceutically acceptable salt thereof.

109. A method of treating a disease of the central nervous in a patient, the method comprising:
i) identifying the disease of the central nervous system as being associated with abnormal activity or abnormal expression of a histone deacetylase (HDAC); and
ii) if the disease of the central nervous system is identified as being associated with abnormal activity or abnormal expression of a histone deacetylase (HDAC), then administering to the patient a therapeutically effective amount of a compound of claim 1, or a pharmaceutically acceptable salt thereof.

110. A method of treating an inflammatory autoimmune disease in a patient, the method comprising:
i) identifying the inflammatory autoimmune disease as being associated with abnormal activity or abnormal expression of a histone deacetylase (HDAC); and
ii) if the inflammatory autoimmune disease is identified as being associated with abnormal activity or abnormal expression of a histone deacetylase (HDAC), then administering to the patient a therapeutically effective amount of a compound of claim 1, or a pharmaceutically acceptable salt thereof.
